SEQUENCE LISTING (17 sequences)

SEQ ID NO: 1 - DNA, 563 bp, Artificial sequence
  Synthetic promotor sequence
acgcgtatcccggcgcgccctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttggctccttcgctttctgggctcagaggctgggaaggggtgggtccgggggcgggctcaggggcgggctcaggggcggggcgggcgcccgaaggtcctccggaagcccggcattctgcacgcttcaaaagcgcacgtctgccgcgctgttctcctcttcctcatctccgggcctttcgacctgcagccgagatctagtactagtgggccaccatgg

SEQ ID NO: 2 - DNA, 1586 bp, Artificial sequence
  Synthetic promotor sequence
acgcgttaacataattttattggaccacgttatttagttgttgggccttgtaaggattaaatgagatcatgcatgtaacactacagtaacagccacatgataaatgtccaaataatatttacctgtgcctggcacagagcaggcactcaaaaaatattttttagagcatgtgacgcgccatgaaccagaggagccactttaaatggacaaacggggatctcatttttttttttttttttaatggtgagacaagccctgagaaggcaaatggactgcctaaagctacacaggtcagcggggcaggtaaatctaaattggcaaagtaagggctgagcgaacagactccgacactagaaggcagagctgaactaacttctgctaaggtcccgcctctgccgctttgtcccgcccttatcttctcccctcctccagcgcctcattcccttttcgctcgccccggccgtgctgaagcaacttccgccctgagaagggtggggcttccgtctcccgctctcgcgactcctggcggtgaaggacggaagatgatagccacatttcttcctcgcccttcccctaggttccctgtcacagttccgcccttactactcccacttccggccagggaacagccacttccacccggaaaaggggttgttccgccgtggggcgccagctgtggcccacccatcctgccccgtactttcgccatcatagtattctccaccactgttccttccagccacgaacgacgcaaacgaagccaagttcccccagctccgaacaggagctctctatcctctctctattacactccgggagaaggaaacgcgggaggaaacccaggcctccacgcgcgaccccttggccctcccctttacctctccacccctcactagacaccctcccctctaggcggggacgaactttcgccctgagagaggcggagcctcagcgtctaccctcgctctcgcgagctttcggaactctcgcgagaccctacgcccgacttgtgcgcccgggaaaccccgtcgttccctttcccctggctggcagcgcggaggccgcacggtaagcgggggctccgagctggaccgggcgcgaggtggcagggccggacgccgaagcctcagagcgcgtgcctgagggccccgaggcgcccggcgcgggcccgtcccgccccctagagccgcggccacgtgcgagcggcaggcccggacatgcccggtcagcgccgtccgggaaccgagcgtgggccccggggggcagcggcggggtgcgtggggcgtccggagtcccggggctggggagtggggtcgcgcaggatcctcacacgcaggggccgggctctgttagtgcgatccagagaggccgtgggcgtcggtgagctccttcagacccgcaggagccggagcccggcgttgaaggggccgtgggaagtaacggggggtaccacggtttaggatgcgctggagcgaaaggattggggtggggtccgtgctcttggcagtcgtctctgccaggcctgtgttcacatgcttgactttctccctcaggtacctggagttactgtaaaagagccaccatgg

SEQ ID NO: 3 - DNA, 2132 bp, Artificial sequence
  Synthetic promotor sequence
acgcgtgctagcccgggctcgagatctctcgggaggctgaggcaggagaattgcttgaacccaggaggcggaggttgcggtgagccgagatcgcgccattgcactacagcctgggcaacgagagcgaaactccgtctcaaaaaaaaaaaaaaaaaaaatcctgagtcccgcttgacaccttttgtcaggcaccaccacctttctgggcgaatgcggtagtaccgtctgctctccctgctgctgtcctgaaatccattcaggcacagcggccgagagctttataataaccgattccaggtgttaggtgctttcccagccccgactcctgcgtcctggacccgcagtcctctgcttaatacctttgctttattagaaaacattctcctctactccgttcagctattcgctgagggcccgccaaccgccagcggttgtcagtggcctagaggcagcggacgcaaacacggggagaggtgcaatcgtctcaagtgactcggcgggcggggcccacaaccggaagcgggtgggcgaccttcacccacgtgcgctgcggcttcgttcgccagcatccaagatggcggcagggcggggcccaaggcgcggcgcgaattgtgacgcaggcgtccggcgtgctccgtgcgcaagcgctttcggcggcgattaggtggtttccggttccgctatcttctttttcttcagcgaggcggccgagctggttggtggcggcggtcgtgcgggttcgcgccgggccgagagcgggttgggggctgcgggaggctgcaggggcctgggcggcagaagaggcggccctgagctggctcatgcgggccagtctcggcagggtggctgggcagggctcgcgaggccacggctcggagcccagaccggggcccaggaggcgagcgccgttttggagaggagcctgcctgctctgcctgccagcgtgaccccacgaggcctcgggcgggaagaggtcctcggggcagatccgagttaatgagagaggggtattgagcgtgtagcgttaactctgccagtcactgcgtcagtcgctttggaaatactaaatttctcgagctgagtcttcatacctggctccattactacgtctgtaaggaggagctggtggtagtgtctgctttttagacttttctttagactatttgtatttttttcagatggagtcttgctctgtcgcctaagctggagttcagtggtgcggtctcggctcactgcaatctccacctcccgggctcgagcgattcttctgcctcagcctcccgagtagctgggattataggcgcctgccaccacgcccagttgatttttgtagttttagtagagacggagtttcaccatgttagccaggctcatcttgaactcttgacctcaaatgatccgtctgcctcggccttccaaagtgctgggattacaggcatgagcccctgcgcccggtcgattctttgtctttttaagtcaacttttatatgtgaacaatgcttggcaggtggttggtagatactaagtgatgttcgtggtttggggtcaaggcaagaagtggggtctggagagttttggtgtaattgagaaggaagctaagagtgttgggtgctccagcttggagttagagaggagagaggctgcgacaggaaggcatgtgtgttgtaggggatggcttcccatccaggctggcagcaggagcagcctgtgcagatcaggacctggctgccgtggaagagggtgggaccgccttcagggaagatggatctagcaagttgaagccaaagggtacttattccatcaggagatactgacgagtccttccgccgctaaacctaaggagaataaccacagtctgtgttcctgaagagcacccgtgcggtcaggagggtggaggacatgtggtctttagttccaggacatgtttagactacaggccagggtgtgtgagaagcctagcagggccaggcttggaggagtgaaaggaagacaggtactggggcaggaccagttggacttggtgcaggcaaagggatagcaactgtggtgtaggcacctgagcttgtgctactcaggcatgcattgctcaccagtctatcctgcctcccttcctcctgcagacgcaaccatgg

SEQ ID NO: 4 - DNA, 2231 bp, Artificial sequence
  Synthetic promotor sequence
acgcgtgctgccctgcactggttcccagagactccctccttcccaggtccaaatggctgcaggagcgaagtgggcggaaaaaaagcgaaccagcttgagaaagggcttgacgtgcctgcgtagggagggcgcatgtccccgtgctccgtgtacgtggcggccgcaggggctagaggggggtcccccccgcaggtactccactctcagtctgcaaaagtgtacgcccgcagagccgccccaggtgcctgggtgttgtgtgattgacgcggggaaggaggggtcagccgatccctccccaaccctccatcccatccctgaggattgggctggtacccgcgtctctcggacaggtcagagcgggtcgccgggtggggtcgctgcaaaaaccctgccccggccgcagccggagaggcggagcctcgcggggagggggcgggaccgccgagacaggcctggaaactgctggaaatgccgcagtgccgccgccgcgccttccgccgcatgtcggcaaagagtccccgccagccccggccggcgccctccccctacgctgagctgcccctcagcgcgaaccctccgcccttcctctactcctgcgagagtcgggatctggggctacccaaggttgggtcccgaatgccagtccctctgtcgggacgcgagatgtgtagggcagatgctaggaagaagattgggtctgggagcggtggtccgcgtggttagctgcctccgctctttttcggtgtcccccccagtcccgcccttgggtgtggggacgcctgccccacaagtgtttagggaggtcagtgggttcctcgcccgtagagacaccgtttatgccaaatgagcactcctcatccccgctcttgatggagtcatgtcctagacgtgaaactatggggctgtgatcacaagcaaatgtgtgggcggatccgttgcttgggttcttccccgccccctccttttttcggaccatgacgtcaaggtgggctggtggcggcaggtgcggggttgacaatcatactcctttaaggcggagggatctacaggagggcggctgtactgtgcttcgccttatatagggcgacttggggcccgcagtagctctctcgagtcactccggcgcagtgttgggactgtctgggtatcggaaagcaagcctacgttgctcactattacgtataatccttttcttttcaaggtaaggctgagatctccgctaggcttctttccctttagtgctgtattcgtgttgtttttgtttttttctgtcctttagggagccttagtctagatgtcggggtggcttgtggataacgctctggatttttatagggtgagggtagtggtgggtgaggttttttgagtcctcctcggttttctctagtgtgtttggggggtggggctttctctcggcgcctgctggccgtagcgaggtgggctgtggggttggggcagtgggcggctggcagctgcacgtggtggccgcgcggcccgggacgctgccatttttgcccctccacttccggacgcggctacggggcgtcggagggggaccgcagggtggcgggggtgcccgctcgggtgactcagcacggccttgggggactggctttgtcacctctcttatcggaatcgattctttgtccggatttaattgctcctccggtgggtatcgtatggatcccaggttattcctccctgccctatggggcaggagtgtcccgcccttggactggtcttaggaactgacacctcagggggagcagtttaaagttagtgccatttttatcttaaactagtcactttgacctcccccaaataaagaactgtaggtagtgattttcacatttaaatttgtgtaaggattacttgggatctctagatacctgggttggaccaacattatgatttttctgccatactaccagatgatgctgaggctgctggtcaccattctttaagtaggtgggttctgtgacatttggttgaagaatatttagcttattttctttttccttctgaattttcaggcctcccacttagtgtgtagtctgagatctttaagagaatgcatttttagtcttgggaagggatagtactccggttaaaccagtctgaactcactgtctaaggtcctaacaaatgatatgacctttaggatttttaaacatggggccttagtgttcttttgtaattaatgagatttttattttaggtcgccaccatgg

SEQ ID NO: 5 - DNA, 2648 bp, Artificial sequence
  Synthetic promotor sequence
acgcgtgctgccctgcactggttcccagagactccctccttcccaggtccaaatggctgcaggagcgaagtgggcggaaaaaaagcgaaccagcttgagaaagggcttgacgtgcctgcgtagggagggcgcatgtccccgtgctccgtgtacgtggcggccgcaggggctagaggggggtcccccccgcaggtactccactctcagtctgcaaaagtgtacgcccgcagagccgccccaggtgcctgggtgttgtgtgattgacgcggggaaggaggggtcagccgatccctccccaaccctccatcccatccctgaggattgggctggtacccgcgtctctcggacaggtcagagcgggtcgccgggtggggtcgctgcaaaaaccctgccccggccgcagccggagaggcggagcctcgcggggagggggcgggaccgccgagacaggcctggaaactgctggaaatgccgcagtgccgccgccgcgccttccgccgcatgtcggcaaagagtccccgccagccccggccggcgccctccccctacgctgagctgcccctcagcgcgaaccctccgcccttcctctactcctgcgagagtcgggatctggggctacccaaggttgggtcccgaatgccagtccctctgtcgggacgcgagatgtgtagggcagatgctaggaagaagattgggtctgggagcggtggtccgcgtggttagctgcctccgctctttttcggtgtcccccccagtcccgcccttgggtgtggggacgcctgccccacaagtgtttagggaggtcagtgggttcctcgcccgtagagacaccgtttatgccaaatgagcactcctcatccccgctcttgatggagtcatgtcctagacgtgaaactatggggctgtgatcacaagcaaatgtgtgggcggatccgttgcttgggttcttccccgccccctccttttttcggaccatgacgtcaaggtgggctggtggcggcaggtgcggggttgacaatcatactcctttaaggcggagggatctacaggagggcggctgtactgtgcttcgccttatatagggcgacttggggcccgcagtagctctctcgagtcactccggcgcagtgttgggactgtctgggtatcggaaagcaagcctacgttgctcactattacgtataatccttttcttttcaaggtaaggctgagatctccgctaggcttctttccctttagtgctgtattcgtgttgtttttgtttttttctgtcctttagggagccttagtctagatgtcggggtggcttgtggataacgctctggatttttatagggtgagggtagtggtgggtgaggttttttgagtcctcctcggttttctctagtgtgtttggggggtggggctttctctcggcgcctgctggccgtagcgaggtgggctgtggggttggggcagtgggcggctggcagctgcacgtggtggccgcgcggcccgggacgctgccatttttgcccctccacttccggacgcggctacggggcgtcggagggggaccgcagggtggcgggggtgcccgctcgggtgactcagcacggccttgggggactggctttgtcacctctcttatcggaatcgatgttaaagccttcttgggtgctttgtttctgtgagggagggttgacggtgtgggaagagagctttcggtctccagcacccgatactccctccttccagatctttcttgcagtcccggtggaggaggggcggggaggggagcaggttctggaagattcatgggctccttcctccgcccttcctcgagagctgagattgttctggaagcttctggattctggcgccccgccccagtgcccggatgctggggcgagggagggtgcactgcggcgccccctcctcgcgtggtcctggccgacgcatgtccggcagtgacgagtgtcggcctggtggctacggccaccatctttcttgggtttggtcctgttctgtaattttgtgctgtgaaggggtcgtggtggagcttttggcttatcgattctttgtccggatttaattgctcctccggtgggtatcgtatggatcccaggttattcctccctgccctatggggcaggagtgtcccgcccttggactggtcttaggaactgacacctcagggggagcagtttaaagttagtgccatttttatcttaaactagtcactttgacctcccccaaataaagaactgtaggtagtgattttcacatttaaatttgtgtaaggattacttgggatctctagatacctgggttggaccaacattatgatttttctgccatactaccagatgatgctgaggctgctggtcaccattctttaagtaggtgggttctgtgacatttggttgaagaatatttagcttattttctttttccttctgaattttcaggcctcccacttagtgtgtagtctgagatctttaagagaatgcatttttagtcttgggaagggatagtactccggttaaaccagtctgaactcactgtctaaggtcctaacaaatgatatgacctttaggatttttaaacatggggccttagtgttcttttgtaattaatgagatttttattttaggtcgccaccatgg

SEQ ID NO: 6 - DNA, 1722 bp, Artificial sequence
  Synthetic promotor sequence
acgcgtagaaggatgtcgttcgctcagccttgcgttccagctaaaataaaactgtgtggggtttccgcctcttttttccaaatttaacctggacacccagctcctctgcagtgtctcccctggaaagttctcgagcgttccccagctttagggccacgcccgccctgagatctgccgagtcattgtccttgtcccgcggccccgggagccccccgcgaccggcctgggaggctcagggaggttgaagggggctgagcaaaggaagccccgtcattacctcaaatgtgacccaaaaataaagacccgtccatctcgcagggtgggccagggcgggtcaggagggaggggagggagaccccgactctgcagaaggcgctcgctgcgtgccccacgtccgccgaacgcggggttcgcgacccgaggggaccgcgggggctgaggggaggggccgcggagccgcggctaaggaacgcgggccgcccacccgctcccggtgcagcggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcagttcttgtttgtggatcgctgtgatcgtcacttggtgagtagcgggctgctgggctggccggggctttcgtggccgccgggccgctcggtgggacggaggcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctctggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttaggtgccaccatgg

SEQ ID NO: 7 - DNA, 1223 bp, Artificial sequence
  Synthetic promotor sequence
acgcgtagcttcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctccagtacgtgattcttgatcccgagctggagccaggggcgggccttgcgctttaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaggatctgcacactggtatttcggtttttgggcccgcggccggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctccagggggctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctggagcttttggagtacgtcgtctttaggttggggggaggggttttatgcgatggagtttccccacactgagtgggtggagactgaagttaggccagcttggcacttgatgtaattctcgttggaatttgccctttttgagtttggatcttggttcattctcaagcctcagacagtggttcaaagtttttttcttccatttcaggtgtcgtgaacacgtggtcgcggccaagatctagtactagtgggccaccatgg

SEQ ID NO: 8 - DNA, 1344 bp, Artificial sequence
  Synthetic promotor sequence
acgcgtgctagcccgggctcgagatctagacccctcctccttctcccgccggaaataccctctttcaggacggcgcgcctgtgcggcgcacgcgcgctcagttacttagcaacctcggcgctaagccaccccaggtggagcccagcaacaacagagccaccgcgtcccccaccaatcagcgccgacctcgccttcgcaggcctaaccaatcagtgccggcgctgcaaggaagtttccagagctttcgaggaaggtttcttcaactcaaattcatccgcctgataattttcttatattttcctaaagaaggaagagaagcgcatagaggagaagggaaataattttttaggagcctttcttacggctatgaggaatttggggctcagttgaaaagcctaaactgcctctcgggaggttgggcgcggcgaactactttcagcggcgcacggagacggcgtctacgtgaggggtgataagtgacgcaacactcgttgcataaatttgcgctccgccagcccggagcatttaggggcggttggctttgttgggtgagcttgtttgtgtccctgtgggtggacgtggttggtgattggcaggatcctggtatccgctaacaggtactggcccgcagccgtaacgaccttggtggggtgtgagaggggggaatgggtgaggtcaaggtggaggcttcttggggttgggtgggccgctgaggggagggcgtgggggaagggagggcgaggtgacgcggcgcttggcttttccggaacagtgggccttgttgacttgaggagggcgagtgcggttggcgcgcgcgcgcgttgacggaaactaacggacgtctaaccgatcggcgattctgtcgagtttacttcgcggggaaggcggaaaagaggtagtttgtgtggtttctggaagcctttactttggaatcccagtgtgagaaaggtgccccttcttgtgtttcaatgggatttttatttcgcgagtcttgtgggtttggttttgttttcagtttgcctaacaccgtgcttaggtttgaggcagattggagttcggtcgggggagtttgaatatccggaacagttagtggggaaagctgtggacgcttggtaagagagcgctctggattttccgctgttgacgttgaaaccttgaatgacgaatttcgtattaagtgacttagccttgtaaaattgaggggaggcttgcggaatattaacgtatttaaggcattttgaaggaatagttgctaattttgaagaatattaggtgtaaaagcaagaaatacaatgatcctgaggtgacacgcttatgttttacttttaaactaggtcaccatgg

SEQ ID NO: 9 - DNA, 1603 bp, Artificial sequence
  Synthetic promotor sequence
acgcgtgctagcccgggctcgagatctaaaagatagatataatccataccatctgttcaatcttgtccttttcaactttttcagggaaacttcccccaggtgatagatggatagatacatgagatagacataacgcatacgatcagttcaatcttatcttttttaactttttcagagaaaacttcccttaagtgaacatttaaatctgaattacgtcctgttaaactgttctccaggaaaatgaaataaaataaatcttcaagtttttgtttacctaacaatttgttgtgtcgaacaaaccttcctacttttcaggtaacaaaatggcagcttaggctagaaagccgctcatattcgcaggtacaagggctgggtaagaacgccccgcctggctgactaacttgagttccgcgctctggacaggaattatgcacagggcgtcgctgtggcactagaaaccccaaagtcacaagcgccccagatccgaccaggatgccgctaccggctacagcccagaggcccgctcctgcggcgcaagcccgccttcctgaagaaaaagcccagtcccggcagcgttcttctccggctccgcccttcttccgctcgactttctttgccattggctgacaacggagtacataaggacgtcatttcctgccgcctgtcttttccgtgctacctgcagaggggtccatacggcgttgttctgggtgagttccgtgtagcgtccctggcgccttccagggctagaaaaatgagcttttcctgctcaaatgaagggtgagaagactagtgatgaaagccggtcagactggatctgtcttccgcccggcgcgccccactttaggcctgcggcccgcacatggccaggctcgggctggcgggttcccagagtgctccgggagcgggtggaggtcgccctccagcggaggctccgagctggggttcggaccaggccgcgggtgggcgggagtgcagaaagcgggctaacatcctgtgttgctatcccttcggagtcccacacggcggtgagtctaggcccaggcgctgatttacaccagctacttgggctggtcgggttttcccttcgcgccgtgcgggtcaggagttaaggttctcgggtttttagacaaacaagtggtgacagcacagcgaagtaattccaaagcatccgcctacaatctgcttgaaaatgtctgaaaacaattcatgccttttttgcctttagtttgcatattccaaacatggctgctcttttgtatctagttgttaacttggcgcatccacaacttttccttaattcctatcttgagaagtgttgaatttccattcgctaatttcgtgtagttttattactcggttactctgccgtccacactatttcctcagtaagatgtgcgctgttccgtaatacacgacatgtatgggttaactttctgtttacccttcactacactgtaagctcaatgcctgacactatagcagatggagtttttggttgcttttaagggtgtgccctacttaactcaatggaatgaaaagaaataggttgctctcttatttcagattcccgtcgtaacttaaagggaaattttcaccatgg

SEQ ID NO: 10 - DNA, 1557 bp, Artificial sequence
  Synthetic promotor sequence
acgcgtgctagcccgggctcgagatcttcatttatttagggagacagaggagtttttagctggattacgttatccatacaaggttggaacataattacagaatttggttacaagaagtgttttttttggtgggtgggcgggggtacagctaacattgttttacgaagagtttaacacgcatgagttgctgtcatctggtgacatcgtttgagtctctctagtcattgaacagaacaagaaaaatcgaattaaatttataatctgaactgaagttatatgtgacccatcacatctctcaagttttaaaatgggtttttttgttgttgttgatggagggggagagggtccagcagcttttaaatgttttcacatcgtgtgttccaaaaataactggttagcctaagtcacttccaccctccaatgttgtgaatgcagtctctagcattcgctatttaatgtcttcttcctgcactatttgagaaatcgcgaggtcgacttaataccgcagtcgccacttcgcggaccggagggcggagtctgcttagttctgaggactgcgtgggtccgcgcagagagctcctgctaggcctgcgcgtcccgttctaaattcttaccctttagtccttgtcaccacccccgccgtgggaacggcctgacagtcactcgtcaaaggaagtggctgccggcagctcttgacccggaatcggatcctagtcccaccccctccgctccaggcttccttctgcaacaggcgtgggtcacgctctcgctcggtctttctgccgccatcttggttccgcgttccctgcacagtaagtactttctgtgccgctactgtctatccgcagccatccgcctttctttcgggctaagccgccccggggactgagagttaaggagagttggaggctttactgggccacagggttcctactcgcccctgggcctccggacaaaatggggtctgcggttggtgtcctggcaaaagcagggtagaagggctgcggggcgggcccagaatccgagcctgcagagatgggagcagttgcagtgttgagggcggaagaggagtgcgtcttgttttgggaactgcttcacaggatccagaaaagggtaaggggtcacagccttagaacctgtaacaccgttctccctgtgacaagcaagtgttggacttaaaacaccgtctttcccctcctggtaccccaaagtcggcaaacgtagtccaggaggccccagcccagccagtgtgagtattaggagtggagggggttcacagtagcgtctgaagtctcccatgatcgagagccagcccgggatcctctccctcgggttgagaatgcagtgggaattgctggccttgatagaggcgtgagggtcacatcatatttacctcttattcccaggtgctttggggaaggttgtcaccaaacaccctcacgatttttttctaacagccctcctcggagtttttaagaatacttatctccgggttgggcaagtgaatgtatcccaatcaatttaggcctcctttttattcccttccttcagccatgg

SEQ ID NO: 11 - DNA, 492 bp, Artificial sequence
  Synthetic promotor sequence
acgcgtagcctgaatagagagagatttaacagtatgaatgaagggaggagtcaagaacagtttaggcttctcgcgtacatgattgggtggccccagatgtcattcacaagtataagccttggaggcggaacataactcgaagaagagcccttttgtgctccgcccatagcgtaggaaggtgtcaattggctgtttcgaggaacgcgccaaaaactgccaagggctgtgggaggtgtgttcttgcgtcatttccgagagacttccgcgccgcagtttccctgcttccccagctccagaacttccggccagcgcagccattttggcttcctgaccttgggctacggctgaccgttttttgtggtgtactccgtgccatcaaatccgtcctgacgccgctgctgctgcggggcttgacaggctcggcccggcggctcccagtgccgcgcgccaagatccattcgttgccgccggaggggaagcttgccaccatgg

SEQ ID NO: 12 - DNA, 2731 bp, Artificial sequence
  Synthetic promotor sequence
acgcgtcgttggcaggtcctgaggcagctggcaagacgcctgcagctgaaagatacaaggccagggacaggacagtcccatccccaggaggcagggagtatacaggctggggaagtttgcccttgcgtggggtggtgatggaggaggctcagcaagtcttctggactgtgaacctgtgtctgccactgtgtgctgggtggtggtcatctttcccaccaggctgtggcctctgcaaccttcaagggaggagcaggtcccattggctgagcacagccttgtaccgtgaactggaacaagcagcctccttcctggccacaggttccatgtccttatatggactcatctttgcctattgcgacacacactcagtgaacacctactacgcgctgcaaagagccccgcaggcctgaggtgcccccacctcaccactcttcctatttttgtgtaaaaatccagcttcttgtcaccacctccaaggagggggaggaggaggaaggcaggttcctctaggctgagccgaatgcccctctgtggtcccacgccactgatcgctgcatgcccaccacctgggtacacacagtctgtgattcccggagcagaacggaccctgcccacccggtcttgtgtgctactcagtggacagacccaaggcaagaaagggtgacaaggacagggtcttcccaggctggctttgagttcctagcaccgccccgcccccaatcctctgtggcacatggagtcttggtccccagagtcccccagcggcctccagatggtctgggagggcagttcagctgtggctgcgcatagcagacatacaacggacggtgggcccagacccaggctgtgtagacccagcccccccgccccgcagtgcctaggtcacccactaacgccccaggccttgtcttggctgggcgtgactgttaccctcaaaagcaggcagctccagggtaaaaggtgccctgccctgtagagcccaccttccttcccagggctgcggctgggtaggtttgtagccttcatcacgggccacctccagccactggaccgctggcccctgccctgtcctggggagtgtggtcctgcgacttctaagtggccgcaagccacctgactcccccaacaccacactctacctctcaagcccaggtctctccctagtgacccacccagcacatttagctagctgagccccacagccagaggtcctcaggccctgctttcagggcagttgctctgaagtcggcaagggggagtgactgcctggccactccatgccctccaagagctccttctgcaggagcgtacagaacccagggccctggcacccgtgcagaccctggcccaccccacctgggcgctcagtgcccaagagatgtccacacctaggatgtcccgcggtgggtggggggcccgagagacgggcaggccgggggcaggcctggccatgcggggccgaaccgggcactgcccagcgtggggcgcgggggccacggcgcgcgcccccagcccccgggcccagcaccccaaggcggccaacgccaaaactctccctcctcctcttcctcaatctcgctctcgctctttttttttttcgcaaaaggaggggagagggggtaaaaaaatgctgcactgtgcggcgaagccggtgagtgagcggcgcggggccaatcagcgtgcgccgttccgaaagttgccttttatggctcgagcggccgcggcggcgccctataaaacccagcggcgcgacgcgccaccaccgccgagaccgcgtccgccccgcgagcacagagcctcgcctttgccgatccgccgcccgtccacacccgccgccaggtaagcccggccagccgaccggggcaggcggctcacggcccggccgcaggcggccgcggccccttcgcccgtgcagagccgccgtctgggccgcagcggggggcgcatggggggggaaccggaccgccgtggggggcgcgggagaagcccctgggcctccggagatgggggacaccccacgccagttcggaggcgcgaggccgcgctcgggaggcgcgctccgggggtgccgctctcggggcgggggcaaccggcggggtctttgtctgagccgggctcttgccaatggggatcgcagggtgggcgcggcggagcccccgccaggcccggtgggggctggggcgccattgcgcgtgcgcgctggtcctttgggcgctaactgcgtgcgcgctgggaattggcgctaattgcgcgtgcgcgctgggactcaaggcgctaactgcgcgtgcgttctggggcccggggtgccgcggcctgggctggggcgaaggcgggctcggccggaaggggtggggtcgccgcggctcccgggcgcttgcgcgcacttcctgcccgagccgctggccgcccgagggtgtggccgctgcgtgcgcgcgcgccgacccggcgctgtttgaaccgggcggaggcggggctggcgcccggttgggagggggttggggcctggcttcctgccgcgcgccgcggggacgcctccgaccagtgtttgccttttatggtaataacgcggccggcccggcttcctttgtccccaatctgggcgcgcgccggcgccccctggcggcctaaggactcggcgcgccggaagtggccagggcgggggcgacctcggctcacagcgcgcccggctattctcgcagctcacagatctagtactagtgggccaccatgg

SEQ ID NO: 13 - DNA, 720 bp, Artificial sequence
  Synthetic promotor sequence
atggtgagcaagggcgccgagctgttcaccggcatcgtgcccatcctgatcgagctgaatggcgatgtgaatggccacaagttcagcgtgagcggcgagggcgagggcgatgccacctacggcaagctgaccctgaagttcatctgcaccaccggcaagctgcctgtgccctggcccaccctggtgaccaccctgagctacggcgtgcagtgcttctcacgctaccccgatcacatgaagcagcacgacttcttcaagagcgccatgcctgagggctacatccaggagcgcaccatcttcttcgaggatgacggcaactacaagtcgcgcgccgaggtgaagttcgagggcgataccctggtgaatcgcatcgagctgaccggcaccgatttcaaggaggatggcaacatcctgggcaataagatggagtacaactacaacgcccacaatgtgtacatcatgaccgacaaggccaagaatggcatcaaggtgaacttcaagatccgccacaacatcgaggatggcagcgtgcagctggccgaccactaccagcagaatacccccatcggcgatggccctgtgctgctgcccgataaccactacctgtccacccagagcgccctgtccaaggaccccaacgagaagcgcgatcacatgatctacttcggcttcgtgaccgccgccgccatcacccacggcatggatgagctgtacaagtaa

SEQ ID NO: 14 - DNA, 717 bp, Artificial sequence
  Synthetic promotor sequence
atggtgagcaagggcgccgagctgttcaccggcatcgtgcccatcctgatcgagctgaatggcgatgtgaatggccacaagttcagcgtgagcggcgagggcgagggcgatgccacctacggcaagctgaccctgaagttcatctgcaccaccggcaagctgcctgtgccctggcccaccctggtgaccaccctgagctacggcgtgcagtgcttctcacgctaccccgatcacatgaagcagcacgacttcttcaagagcgccatgcctgagggctacatccaggagcgcaccatcttcttcgaggatgacggcaactacaagtcgcgcgccgaggtgaagttcgagggcgataccctggtgaatcgcatcgagctgaccggcaccgatttcaaggaggatggcaacatcctgggcaataagatggagtacaactacaacgcccacaatgtgtacatcatgaccgacaaggccaagaatggcatcaaggtgaacttcaagatccgccacaacatcgaggatggcagcgtgcagctggccgaccactaccagcagaatacccccatcggcgatggccctgtgctgctgcccgataaccactacctgtccacccagagcgccctgtccaaggaccccaacgagaagcgcgatcacatgatctacttcggcttcgtgaccgccgccgccatcacccacggcatggatgagctgtacaag

SEQ ID NO: 15 - DNA, 606 bp, Artificial sequence
  Synthetic promotor sequence
atgactgaatacaaaccaactgttagactggcaactagagatgatgttccaagagcagttagaaccctggctgctgcatttgctgactaccctgcaaccagacacactgtggacccagacagacacattgaaagagtgactgaactgcaggagctgttcctgaccagagtgggcctggacattggcaaagtgtgggtggcagatgatggtgctgctgtggcagtgtggaccacccctgaatctgttgaagctggtgcagtgtttgctgagattggcccaagaatggcagaactgtctggcagcagactggcagcacaacagcagatggaaggtctgctggcaccacacagaccaaaagaacctgcttggttcctggcaactgtgggtgtgagccctgaccaccagggtaagggcctgggctctgcagtggtgctgcctggtgtggaagcagctgaaagagcaggtgtgcctgctttcctggagacctcagctccaagaaacctgcctttctatgaaagactgggcttcactgtgactgctgatgtggaagtgccagaaggcccaagaacttggtgcatgactagaaaaccaggtgcttgataatga

SEQ ID NO: 16 - DNA, 606 bp, Artificial sequence
  Synthetic promotor sequence
atgactgaatacaaaccaactgttagactggcaactagagatgatgttccaagagcagttagaaccctggctgctgcatttgctgactaccctgcaaccagacacactgtggacccagacagacacattgaaagagtgactgaactgcaggagctgttcctgaccagagtgggcctggacattggcaaagtgtgggtggcagatgatggtgctgctgtggcagtgtggaccacccctgaatctgttgaagctggtgcagtgtttgctgagattggcccaagaatggcagaactgtctggcagcagactggcagcacaacagcagatggaaggtctgctggcaccacacagaccaaaagaacctgcttggttcctggcaactgtgggtgtgagccctgaccaccagggtaagggcctgggctctgcagtggtgctgcctggtgtggaagcagctgaaagagcaggtgtgcctgctttcctggagacctcagctccaagaaacctgcctttctatgaaagactgggcttcactgtgactgctgatgtggaatgcccaaaggacagagcaacttggtgcatgactagaaaaccaggtgcttgataatga

SEQ ID NO: 17 - DNA, 1977 bp, Artificial sequence
  Synthetic promotor sequence
aaatcctgagtcccgcttgacaccttttgtcaggcaccaccacctttctgggcgaatgcggtagtaccgtctgctctccctgctgctgtcctgaaatccattcaggcacagcggccgagagctttataataaccgattccaggtgttaggtgctttcccagccccgactcctgcgtcctggacccgcagtcctctgcttaatacctttgctttattagaaaacattctcctctactccgttcagctattcgctgagggcccgccaaccgccagcggttgtcagtggcctagaggcagcggacgcaaacacggggagaggtgcaatcgtctcaagtgactcggcgggcggggcccacaaccggaagcgggtgggcgaccttcacccacgtgcgctgcggcttcgttcgccagcatccaagatggcggcagggcggggcccaaggcgcggcgcgaattgtgacgcaggcgtccggcgtgctccgtgcgcaagcgctttcggcggcgattaggtggtttccggttccgctatcttctttttcttcagcgaggcggccgagctggttggtggcggcggtcgtgcgggttcgcgccgggccgagagcgggttgggggctgcgggaggctgcaggggcctgggcggcagaagaggcggccctgagctggctcatgcgggccagtctcggcagggtggctgggcagggctcgcgaggccacggctcggagcccagaccggggcccaggaggcgagcgccgttttggagaggagcctgcctgctctgcctgccagcgtgaccccacgaggcctcgggcgggaagaggtcctcggggcagatccgagttaatgagagaggggtattgagcgtgtagcgttaactctgccagtcactgcgtcagtcgctttggaaatactaaatttctcgagctgagtcttcatacctggctccattactacgtctgtaaggaggagctggtggtagtgtctgctttttagacttttctttagactatttgtatttttttcagatggagtcttgctctgtcgcctaagctggagttcagtggtgcggtctcggctcactgcaatctccacctcccgggctcgagcgattcttctgcctcagcctcccgagtagctgggattataggcgcctgccaccacgcccagttgatttttgtagttttagtagagacggagtttcaccatgttagccaggctcatcttgaactcttgacctcaaatgatccgtctgcctcggccttccaaagtgctgggattacaggcatgagcccctgcgcccggtcgattctttgtctttttaagtcaacttttatatgtgaacaatgcttggcaggtggttggtagatactaagtgatgttcgtggtttggggtcaaggcaagaagtggggtctggagagttttggtgtaattgagaaggaagctaagagtgttgggtgctccagcttggagttagagaggagagaggctgcgacaggaaggcatgtgtgttgtaggggatggcttcccatccaggctggcagcaggagcagcctgtgcagatcaggacctggctgccgtggaagagggtgggaccgccttcagggaagatggatctagcaagttgaagccaaagggtacttattccatcaggagatactgacgagtccttccgccgctaaacctaaggagaataaccacagtctgtgttcctgaagagcacccgtgcggtcaggagggtggaggacatgtggtctttagttccaggacatgtttagactacaggccagggtgtgtgagaagcctagcagggccaggcttggaggagtgaaaggaagacaggtactggggcaggaccagttggacttggtgcaggcaaagggatagcaactgtggtgtaggcacctgagcttgtgctactcaggcatgcattgctcaccagtctatcctgcctcccttcctcctgcagacgcaaccatgg

(19) United States
(12) Patent Application Publication
(10) Pub. No.: US 2022/0389436 A1
(43) Pub. Date: Dec. 8, 2022

(54) METHODS TO PREVENT RAPID SILENCING OF GENES IN PLURIPOTENT STEM CELLS

(71) Applicant: FUJIFILM Cellular Dynamics, Inc., Madison, WI (US)

(72) Inventors: Sarah DICKERSON, Madison, WI (US); Sarah BURTON, Madison, WI (US); Christie MUNN, Madison, WI (US); Madelyn GOEDLAND, Madison, WI (US); Michael MCLACHLAN, Madison, WI (US); Deepika RAJESH, Madison, WI (US); Tom BURKE, Madison, WI (US)

(73) Assignee: FUJIFILM Cellular Dynamics, Inc., Madison, WI (US)

(21) Appl. No.: 17/826,112

(22) Filed: May 26, 2022

Related U.S. Application Data
(60) Provisional application No. 63/193,472, filed on May 26, 2021.

(51) Int. Cl. C12N 15/79

(52) U.S. Cl. CPC C12N 15/79

(57) ABSTRACT

Provided herein are methods of producing cell lines with stable expression of a transgene by removal of CpG motifs. In further methods, there are provided methods for cell lines with stable expression of a transgene by driving expression by novel promoters or by tagging endogenous genes.

### PRIORITY CLAIM

This application claims benefit of priority to U.S. Provisional Application Ser. No. 63/193,472, filed May 26, 2021, the entire contents of which are hereby incorporated by reference.

### INCORPORATION OF SEQUENCE LISTING

The sequence listing that is contained in the file named “CDINP0103US.TXT”, which is 34,000 bytes (as measured in Microsoft Windows®) and was created on May 26, 2022, is filed herewith by electronic submission and is incorporated by reference herein.

### BACKGROUND

### 1. Field

The present disclosure relates generally to the field of stem cell biology. More particularly, it concerns methods for the codon optimization of genes in induced pluripotent stem cells to reduce rapid silencing of genes.

### 2. Description of Related Art

Studies have shown that there are significant differences in the performance of seemingly identical cell lines (Kyttala, 2016). These differences detected when comparing multiple clones from the same donor are referred to as “clone to clone variation”. These clones are believed, and in some cases confirmed, to contain identical DNA sequences. Inconsistent yield and purity in differentiation batches of the same cell line is referred to as “batch to batch variation”. In many cases, differences in differentiation performance have been attributed to epigenetic modifications; however, there is an unmet need to identify the specific epigenetic mechanisms and methods for altering these epigenetic mechanisms to prevent variations in cell lines.

### SUMMARY

In a first embodiment, the present disclosure provides an isolated cell line engineered to express at least one transgene wherein the at least one transgene (a) is under the control of a promoter having at least 90% (e.g., at least 91%, 92%, 93%, 94%, 95%, 96%, 97%, 98%, or 99%) sequence identity to SEQ ID NOs:1-12 or 17; (b) is under the control of an endogenous gene selected from the group consisting of HSP90AB1, ACTB, CTNNB1, MYL6, UBA52, CAG, RPS, and UBC; and/or (c) is encoded by a sequence modified to remove CpG motifs to provide for stable expression. In certain aspects, the cell line is an induced pluripotent stem cell (iPSC) line.

In some aspects, the sequence modified to remove CpG motifs to provide for stable expression has at least 90% (e.g., at least 91%, 92%, 93%, 94%, 95%, 96%, 97%, 98%, or 99%) sequence identity to SEQ ID NO:14 or SEQ ID NO:16. In certain aspects, the sequence modified to remove CpG motifs to provide for stable expression is SEQ ID NO:14 or SEQ ID NO:16.

In some aspects, at least one transgene wherein the at least one transgene (a) is under the control of a promoter having at least 90% (e.g., at least 91%, 92%, 93%, 94%, 95%, 96%, 97%, 98%, or 99%) sequence identity to SEQ ID NOs:1-12 or 17; and/or (b) is under the control of an endogenous gene selected from the group consisting of HSP90AB1, ACTB, CTNNB1, MYL6, UBA52, CAG, RPS, and UBC. In particular aspects, the at least one transgene is encoded by a sequence modified to remove CpG motifs to provide for stable expression.

In certain aspects, the at least one transgene is encoded by a sequence modified to remove CpG motifs to provide for stable expression and is under the control of a promoter having at least 90% (e.g., at least 91%, 92%, 93%, 94%, 95%, 96%, 97%, 98%, or 99%) sequence identity to SEQ ID NOs:1-12 or 17. In some aspects, the at least one transgene is encoded by a sequence modified to remove CpG motifs to provide for stable expression and is under the control of an endogenous gene selected from the group consisting of HSP90AB1, ACTB, CTNNB1, MYL6, UBA52, CAG, RPS, and UBC. In specific aspects, the at least one transgene is encoded by a sequence modified to remove CpG motifs to provide for stable expression and is under the control of an endogenous gene selected from the group consisting of HSP90AB1, ACTB, CTNNB1, and MYL6.

In further aspects, the cell line is engineered to express at least a first transgene and a second transgene. In some aspects, the first transgene is under the control of a promoter having at least 90% (e.g., at least 91%, 92%, 93%, 94%, 95%, 96%, 97%, 98%, or 99%) sequence identity to SEQ ID NOs:1-12 or 17 and the second transgene is under the control of an endogenous gene selected from the group consisting of HSP90AB1, ACTB, CTNNB1, MYL6, UBA52, CAG, RPS, and UBC. In other aspects, the first transgene is under the control of a promoter having at least 90% (e.g., at least 91%, 92%, 93%, 94%, 95%, 96%, 97%, 98%, or 99%) sequence identity to SEQ ID NOs:1-12 or 17 and the second transgene is under the control of an endogenous gene selected from the group consisting of HSP90AB1, ACTB, CTNNB1, and MYL6. In some aspects, the first transgene and/or second transgene are encoded by a sequence modified to remove CpG motifs for stable expression. In specific aspects, at least 50 percent, such as at least 70 percent, 80 percent, 90 percent, 95 percent, 96 percent, 97 percent, 98 percent or 99 percent, of the CpG motifs are removed. In particular aspects, all CpG motifs are removed. In some aspects, the CpG motif codons are replaced with codons that are not rare and/or do not generate a mononucleotide stretch. In particular aspects, the CpG motif codons are replaced with corresponding codons in Table 1.

In some aspects, the promoter is a response element. In certain aspects, the promoter is driven by a response element.

In some aspects, the transgene is a reporter gene or selection marker. In certain aspects, the reporter gene is a fluorescent or luminescent protein, such as luciferase, green fluorescent protein (GFP) or red fluorescent protein (RFP). In certain aspects, the at least one transgene is a selection marker, such as puromycin, neomycin, or blasticidin. In particular aspects, the at least one transgene is a suicide gene. In some aspects, the at least one transgene is thymidine kinase, TET, or myoblast determination protein 1 (MYOD1).

In particular aspects, the cell line has stable expression of the transgene for at least 30 days, such as at least 2 months, 3 months, 4 months, 5 months or longer. In particular aspects, the cell line has stable expression of the transgene over six months, such as over one year, over two years, or over three years.

In some aspects, the at least one transgene is encoded by an expression cassette. In certain aspects, the at least one transgene is introduced into the cell line by electroporation or lipofection. In specific aspects, the expression cassette is inserted at a genomic safe harbor site, such as the PPP1R12C (AAVS1) locus or ROSA locus.

In certain aspects, the promoter has at least 90% (e.g., at least 91%, 92%, 93%, 94%, 95%, 96%, 97%, 98%, or 99%) sequence identity to SEQ ID NO: 2, 3, 4, 6, or 17. In some aspects, the promoter comprises SEQ ID NO: 2, 3, 4, 6, or 17.

In particular aspects, the method comprises gene editing, specifically the transgene comprises gene editing, such as TALEN-mediated gene editing, CRISPR-mediated gene editing, or ZFN-mediated gene editing.

A further embodiment provides a method to prevent silencing of transgene expression in an engineered cell line comprising optimizing the transgene sequence to remove CpG motifs.

In some aspects, optimizing comprises replacing essentially all CpG motif codons. In certain aspects, optimizing comprises replacing at least 50 percent, such as at least 70 percent, 80 percent, 90 percent, 95 percent, 96 percent, 97 percent, 98 percent or 99 percent, of the CpG motifs are removed. In particular aspects, all CpG motifs are removed. In specific aspects, the CpG motif codons are replaced with codons that are not rare and/or do not generate a mononucleotide stretch. In some aspects, the CpG motif codons are replaced with corresponding codons in Table 1. In specific aspects, the transgene sequence optimized to remove CpG motifs comprises a percent GC content substantially similar to the percent GC content of the wild-type transgene sequence.

In some aspects, the transgene sequence is a reporter gene, such as a fluorescent protein, such as GFP or RFP.

In certain aspects, the transgene is under the control of a constitutive promoter. In some aspects, the constitutive promoter has expression in substantially all cell types. In particular aspects, the constitutive promoter has expression in essentially all cell types. In certain aspects, the constitutive promoter has expression in all cell types.

In particular aspects, the transgene is under the control of an inducible promoter. In some aspects, the transgene is under the control of an EEF1A1 promoter.

In additional aspects, the method further comprises treating the cell line with sodium butyrate, VPA, or TSA. In specific aspects, the sodium butyrate is added at a concentration of 0.25 mM to 0.5 mM.

In some aspects, the cell line is an iPSC line. In certain aspects, the method further comprises differentiating the iPSC line. In some aspects, the iPSC line is differentiated to mature cells, such as, but not limited to, hematopoietic precursor cells, neural precursor cells, GABAergic neurons, macrophages, microglia, or endothelial cells.

Another embodiment provides an expression vector comprising a promoter having at least 90% (e.g., at least 91%, 92%, 93%, 94%, 95%, 96%, 97%, 98%, or 99%) sequence identity to SEQ ID NOs: 1-12 or 17. In some aspects, the promoter has at least 90% (e.g., at least 91%, 92%, 93%, 94%, 95%, 96%, 97%, 98%, or 99%) sequence identity to SEQ ID NO: 2, 3, 4, 6, or 17. In certain aspects, the promoter comprises SEQ ID NO: 2, 3, 4, 6, or 17. In particular aspects, the expression vector is a pGL3 plasmid vector. In some aspects, the vector encodes a transgene under the control of the promoter. In particular aspects, the transgene is a reporter gene, such as a fluorescent or luminescent protein, such as luciferase, green fluorescent protein (GFP) or red fluorescent protein (RFP).

A further embodiment provides a method of generating a cell line with stable transgene expression comprising engineering the cell line to express a vector of the present embodiments (e.g., comprising a promoter having at least 90% (e.g., at least 91%, 92%, 93%, 94%, 95%, 96%, 97%, 98%, or 99%) sequence identity to SEQ ID NOs: 1-12 or 17), wherein the vector encodes said transgene. In some aspects, the cell line is a pluripotent cell line, such as an iPSC line.

In some aspects, the method comprises integrating the vector at the AAVS1 locus on chromosome 19. In certain aspects, integrating comprises gene editing, such as CRISPR-mediated gene editing, TALEN-mediated gene editing, or ZFN-mediated editing.

In further aspects, the method further comprises differentiating the cell line. In some aspects, the cell line is differentiated to hematopoietic precursor cells, neural precursor cells, GABAergic neurons, macrophages, microglia, or endothelial cells. In particular aspects, the cell line is cultured for at least 30 days, such as at least 2 months, 3 months, 4 months, 5 months or longer. In particular aspects, the cell line is cultured for over six months, such as over one year, over two years, or over three years. In particular aspects, the cell line has stable expression of the transgene for at least 30 days, such as at least 2 months, 3 months, 4 months, 5 months or longer. In particular aspects, the cell line has stable expression of the transgene over six months, such as over one year, over two years, or over three years. In some aspects, the cell line is cultured for at least six months. In certain aspects, the cell line has stable expression of the transgene at six months.

Another embodiment provides an isolated pluripotent cell line comprising an expression vector of the present embodiments (e.g., comprising a promoter having at least 90% (e.g., at least 91%, 92%, 93%, 94%, 95%, 96%, 97%, 98%, or 99%) sequence identity to SEQ ID NOs: 1-12 or 17).

A further embodiment provides a method of generating a cell line with stable expression of an exogenous transgene comprising engineering the cell line to express the transgene under the control of an endogenous gene, wherein the endogenous gene is HSP90AB1, ACTB, CTNNB1, MYL6, UBA52, CAG, RPS, and UBC, such as HSP90AB1, ACTB, CTNNB1, or MYL6.

In some aspects, engineering comprises gene editing, such as TALEN-mediated gene editing, CRISPR-mediated gene editing, or ZFN-mediated gene editing. In some aspects, the transgene is a reporter gene, selection marker, or suicide gene.

In certain aspects, the cell line is a pluripotent cell line, such as an iPSC line.

Another embodiment provides isolated cell line with endogenous HSP90AB1, ACTB, CTNNB1, MYL6, UBA52, CAG, RPS, and UBC tagged with a transgene. In some aspects, the transgene is a reporter gene, selection marker, or suicide gene. In certain aspects, the cell line is a pluripotent cell line, such as an iPSC line.

Further provided herein is an assay for detecting a cell comprising culturing a cell line of the present embodiments and measuring the expression of a reporter gene. Also provided herein is the use of a cell line of the present embodiments for a cellular assay, such as a cell viability assay, or an assay for screening candidate agents. In some aspects, the assay is a high-throughput assay. In certain aspects, the cellular assay comprises measuring expression of a reporter gene.

Another embodiment provides a composition comprising the cell line of the present embodiments for use in a cellular assay.

Other objects, features and advantages of the present disclosure will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The patent or application file contains at least one drawing executed in color. Copies of this patent or patent application publication with color drawings(s) will be provided by the Office upon request and payment of the necessary fee.

The following drawings form part of the present specification and are included to further demonstrate certain aspects of the present invention. The invention may be better understood by reference to one or more of these drawings in combination with the detailed description of specific embodiments presented herein.

FIGS. 1A-1D: ZsGreen expression driven by EEF1A1p is mottled in iPSCs. iPSCs 01278.103 (FIG. 1A, 1B) and 01279.107 (FIG. 1C, 1D) were engineered with EEF1A1p-ZsGreen at the PPP1R12C locus. Brightfield and Fluorescence (GFP) microscopy were used to capture the GFP expression in the cells 11 passages post-engineering (FIG. 1A-1B) or 18 passages post-engineering (FIGS. 1C-1D).

FIG. 2: Codon optimization of AcGFP1 DNA sequence (SEQ ID NO:13) resulted in a CpG-free AcGFP1 DNA sequence (SEQ ID NO:14).

FIGS. 3A-3B: CpG-free AcGFP1 expression is stable while AcGFP1 expression is not maintained overtime. The percent GFP expression in five clones targeted with EEF1A1p-CpG-free-AcGFP1 (FIG. 3A) and nine clones targeted with EEF1A1p-AcGFP1 (FIG. 3B) at the AAVS1 locus was monitored overtime.

FIGS. 4A-4C: Rapid silencing of AcGFP1 in iPSCs. Depiction of engineering iPSCs at the PPP1R12C locus (AAVS1 safe harbor) with three cassettes EEF1A1p-mRFP1+PGKp-Puro, EEF1A1p-AcGFP1 or EEF1A1p-CpG-Free AcGFP1. Noted within the figure are the engineered iPSC ID numbers for cell lines 8717 and 9650 which are used in further experiments throughout the document (FIG. 4A). AcGFP1 expressing clones were picked and expanded but did not retain consistent expression. After two months in culture, AcGFP1 engineered iPSCs were bulk sorted for AcGFP1 expression. Brightfield and Fluorescence (GFP) microscopy was used to capture the GFP expression in the cells 12 days post-sorting (FIG. 4B) or 23 days post-sorting (FIG. 4C). Similar silencing was observed with other green fluorescent proteins, including monomer mNeonGreen and tetramer ZsGreen.

FIGS. 5A-5B: Silenced transgene be reactivated with NaBut treatment. A small number of cells were silenced in the CpG-free AcGPF1 cultures (<3% of cells, FIG. 3A). These silenced cells were single cell sorted and expanded to further investigate their silencing and research methods for overcoming the silencing. Two months after sorting for no GFP expression, silenced CpG-free AcGFP1 clones were treated with 1 mM, 0.5 mM, or 1 μM of NaBut. After nine days of NaBut treatment the cells were assayed for % GFP expression by flow cytometry, a dose-dependent reactivation of CpG-free AcGFP1 was observed. After a successful pilot experiment, the duration of NaBut treatment was increased to 46 days with NaBut treatment doses of 0.25 mM and 0.5 mM and the GFP expression levels were monitored over time by fluorescent microscopy and flow cytometry. The initial results were confirmed (FIGS. 5A and 5B, dark blue bar: day 8 of treatment). A dose dependent effect of NaBut treatment was evident through the duration of the experiment (FIGS. 5A and 5B).

FIG. 6: iPSC 9650 (CpG-free-AcGFP1 at AAVS1) differentiation. iPSC 9650 maintained GFP expression throughout hepatocyte differentiation (measured by CXCR4, AAT and ALB expression) and induced neuron (iN) differentiation (measured by TUJ expression).

FIGS. 7A-7C: Plasmids for 1069: WT PuroR (FIG. 7A), 1362: CpG-free PuroR1 (FIG. 7B) and 1363: CpG-free PuroR1 (FIG. 7C).

FIG. 8: Schematic description of the protocol to generate endothelial cells from iPSC 9650-GFP engineered with CpG-freeAcGFP1 at AAVS1.

FIG. 9: Hypoxic acclimatized iPSCs were plated on Purecoat Amine plates to initiate the generation of hematoendothelial cells for 6 days. A representative photograph of iPSC derived hematoendothelial cells on day 6 of differentiation reveals the presence of hematoendothelial colonies in two-dimensional format retaining the expression of GFP.

FIG. 10: Morphology of 9650-GFP derived endothelial cells at passage 2 in culture revealing an overlap of GFP/BF using a 4x objective.

FIG. 11: Purity of endothelial cells derived from 9650-GFP iPSCs. Hypoxic acclimatized iPSCs were plated on Purecoat Amine plates to initiate the generation of hematoendothelial cells and subsequently replated to generate pure endothelial cells that can be propagated over multiple passages. The purity of endothelial cells was quantified at passage by staining for the co-expression of CD31, CD144 and CD105 by flow cytometry.

FIG. 12: Hypoxic acclimatized iPSCs were plated on Purecoat Amine plates to initiate the generation of hematoendothelial cells and subsequently replated to generate pure endothelial cells that can be propagated over multiple passages. The intensity of GFP expression was quantified by flow cytometry over multiple passages.

FIG. 13: Schematic description of the protocol to generate hematopoietic precursor cells (HPCs) from iPSCs.

FIGS. 14A-14C: Hypoxically acclimatized iPSCs were harvested and differentiated to HPCs in a 3D aggregate format over a period of 13-15 days. At the end of the HPC differentiation process the cells were harvested, the purity of HPCs was quantified by staining for the expression of CD34, CD45, CD31, CD41 and CD235 expression along with GFP (FIG. 14A) or RFP (FIG. 14C) expression to show the retention of fluorescence in end stage HPCs. Co-expression of GFP with CD34 post-MACS separation is greater than 90% (FIG. 14B).

FIG. 15. Efficiency of generating HPCs: 1 input iPSC gave rise to 0.766 and 0.225 HPCs for 8717 and 9650, respectively

FIG. 16: Schematic of generation of microglia from HPCs.

FIGS. 17A-17B: Phase and fluorescence images from microglia differentiation of lines 9650-GFP (FIG. 17A) and 8717-RFP (FIG. 17B).

FIG. 18: Efficiency of generating hematopoietic precursor cells (HPCs). CD34+MACs sorted 9650-GFP derived HPCs and unsorted 8717-RFP were differentiated to Microglia. The total viable number of input HPCs and output Microglia was quantified. The process efficiency was calculated based on the purity and absolute number of CD34+positive cells present on day 23 of Microglia differentiation divided by the absolute number of input viable HPCs.

FIGS. 19C-19D: Purity profile of day 23 microglia generated from 8717-RFP (FIG. 19A) and 9650-GFP (FIG. 19C) iPSCs, respectively. The end stage microglia were harvested and stained for the presence of PU.1, IBA, CX3CR, TREM2 and P2RY12 expression were quantified by flow cytometry. The co expression of the markers was quantified along with the retention of GFP or RFP in end stage cells (FIGS. 19B, 19D).

FIG. 20: Schematic representation of generating end stage macrophages from HPCs.

FIG. 21: HPC derived from 8717-RFP were differentiated further along to generate end stage Macrophages. Purity assessment of end stage macrophages was quantified by staining for the presence of CD68 expression on days 44 and 51 of the differentiation process.

FIG. 22: Phase and fluorescent images of line 8717-RFP line during different days of the Macrophage differentiation process. The images were captured at 10× magnification.

FIG. 23: 8717-RFP iPSC derived HPCs were differentiated to end stage Macrophages. The total viable number of input HPCs and output Macrophages was quantified. The process efficiency was calculated based on the purity and absolute number of CD68+positive cells present on day 51 of Macrophage differentiation divided by the absolute number of input viable HPCs.

FIG. 24: Retaining the presence of the engineered fluorochrome throughout the differentiation process. 9650-GFP and 8717-RFP iPSCs retained the presence of the fluorochromes throughout the differentiation of iPSCs to HPCs and further along to generate pure end stage Microglia and Macrophages.

FIG. 25: A schematic description of the method to generate Neural Precursor Cells (NPCs) form iPSC without using dual SMAD inhibition. The various steps involved, and the composition of the medias used, are described.

FIGS. 26A-26B: (FIG. 26A) Visualization of Red and Green Fluorescence during 2D pre-conditioning stage of NPC differentiation process. FIG. 26B captures the fluorescence of end stage 3D NPC cultures prior to the harvest. All images at taken using 4X objective.

FIG. 27: Quantification of purity post thaw in 8717-RFP and 9650-GFP derived NPCs. NPCs were thawed and stained for the presence of SSEA4, CD56 and CD15 expression using the relevant isotype controls.

FIG. 28: Differentiation protocol of NPCs to GABAergic Neurons. NPCs were placed in a 3D differentiation culture and transitioned to 2D culture on PLO-Laminin coated plates. End stage neurons were harvested at 18 days and the purity of Nestin and β-Tubulin 3 was quantified by flow cytometry.

FIGS. 29A-29B: Bright field and fluorescence images taken at Day 2 (3D) (FIG. 29A) and Day 18 (2D) (FIG. 29B) of GABAergic neuron differentiation. 3D cultures in ULA T25 Flask and 2D cultures on 6 well PLO-Laminin coated plates. All images taken at 10× magnification.

FIG. 30: Retention of GFP and RFP expression in undifferentiated engineered iPSCs and in end stage neuronal cultures on Day 13 and Day 18 of GABAergic differentiation. Day 13 samples were stained prior to plating onto PLO-Laminin and Day 18 cultures were stained at the end of the GABAergic Neuron differentiation.

FIG. 31: GABAergic neurons derived from 9650-GFP and 8717-RFP iPSCs cultures on day 18 of differentiation were harvested and stained for the Nestin and β-Tubulin purity by flow cytometry. The co-expression of GFP or RFP along with Nestin and tubulin in end stage cultures was quantified.

FIGS. 32A-32B: (FIG. 32A) Normalized luciferase (Firefly/Renilla ratio, normalized to EEF1A1=100%) are shown (HSP90AB 1de1400 promoter and HSP90AB1 promoter had expression around 66% and 75% of EEF1A1). (FIG. 32B) Plasmid design using CAG promoter as an example for control of ZsGreen fluorescent protein and being targeted to the AAVS1 (PPP1R12C) safe harbor locus on chromosome 19 in human iPSC.

FIG. 33: Engineered iPSC lines expressing ZsGreen (ZsG) fluorescent protein were maintained in culture for up to seven months (E8 media/vitronectin coated plates) and periodically checked for green expression using flow cytometry on an Accuri C6 instrument (BD). Most clones maintained a consistent flow profile over time, apart from one of the RPS19 promoter clones (5363), which showed many cells with lowered fluorescence at the August time point. The graph shows median fluorescence levels normalized to unengineered iPSC=1.

FIGS. 34A-34B: (FIG. 34A) Flow cytometry plots for the ZsGreen (ZsG) engineered iPSC lines. (FIG. 34B) At day 21 of differentiation, all cells had a visible neuronal phenotype. Flow cytometry shows many cells with diminishing fluorescence for the CAG, UBC(v1), and HSP90AB 1de1400 promoters. The UBCv2, UBA52, and RPS19 promoters showed tight and stable expression, as did the tagged genes HSP90AB1, CTNNB1, and MYL6.

### DESCRIPTION OF ILLUSTRATIVE EMBODIMENTS

DNA methylation plays an important role in modulating the expression of genes including induction transcriptional repression, prevention of transcription factor binding to DNA, requirement for some transcription factor binding to DNA, recruitment of HDAC complexes, X-chromosome inactivation, and the immunogenicity of CpG motifs, such as TLR9. DNA methylation in mammalians occurs when a methyl group is added by a methyltransferase enzyme to the fifth carbon of cytosine (5-mC) in cytosine phosphate-guanine (CpG). DNMT3A and DNMT3B (DNA methyltransferases) are responsible for de novo methylation (i.e., methylating previously unmethylated DNA) and DNMT3B has been shown to be turned on in iPSCs. DNMT1 is responsible for methylating hemi-methylated DNA after replication and is characterized as a maintenance methyltransferase. Demethylation studies have emerged more recently in which Gadd45a has been identified as an important player in DNA demethylation in DNA repair and TET and TDG in oxidation and excision of 5-mC in DNA.

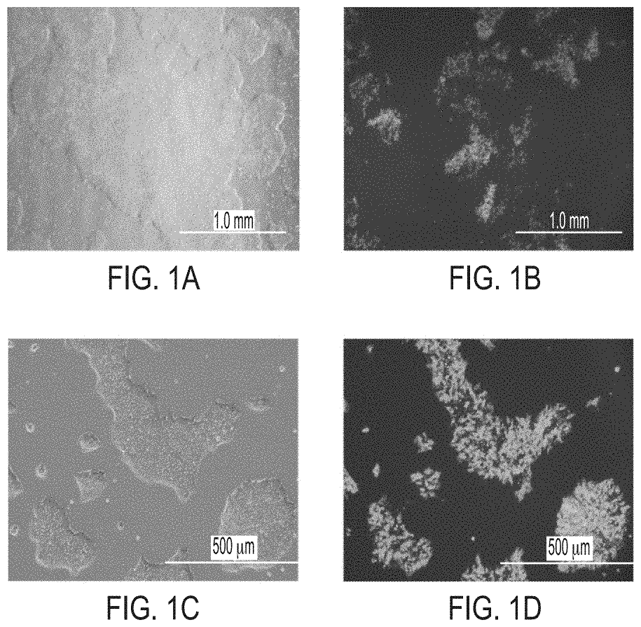

The addition of transgenes through genome engineering into iPSCs has afforded the opportunity to monitor the expression of transgenes over time and through the differentiation process. Green Fluorescent Protein (GFP) and Red Fluorescent Protein (RFP) are widely used for the generation of fusion proteins without significantly interfering with native protein assembly and function, making them powerful tools for in vivo analyses and biomarkers to monitor progenitor populations and determine the kinetics of emerging cell lineages. As evidenced by the lack of iPSCs or differentiated cells expressing green fluorescent protein (GFP) on the market, there is a need for methods to maintain transgene expression through extensive passaging and differentiation. Specifically, FIG. 1 shows mottled expression of GFP in iPSCs.

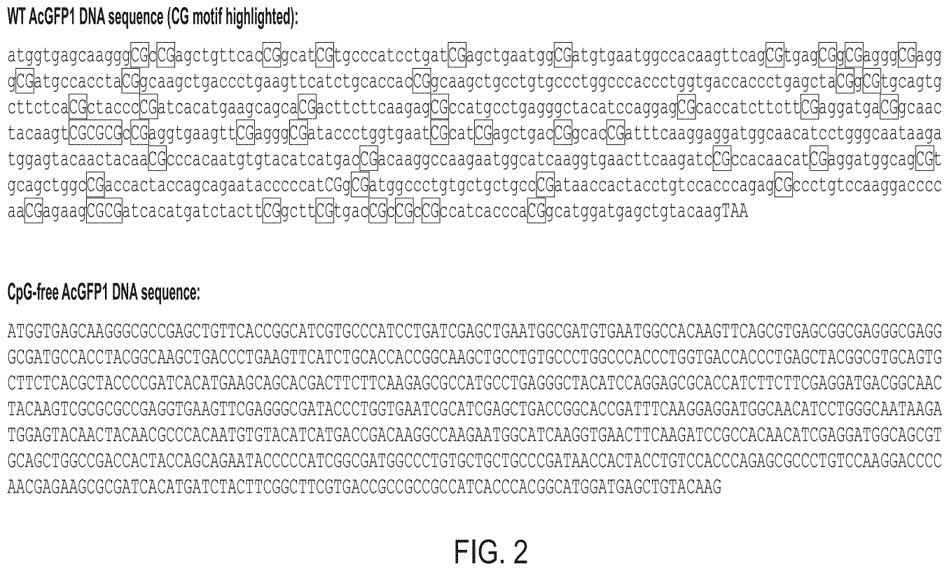

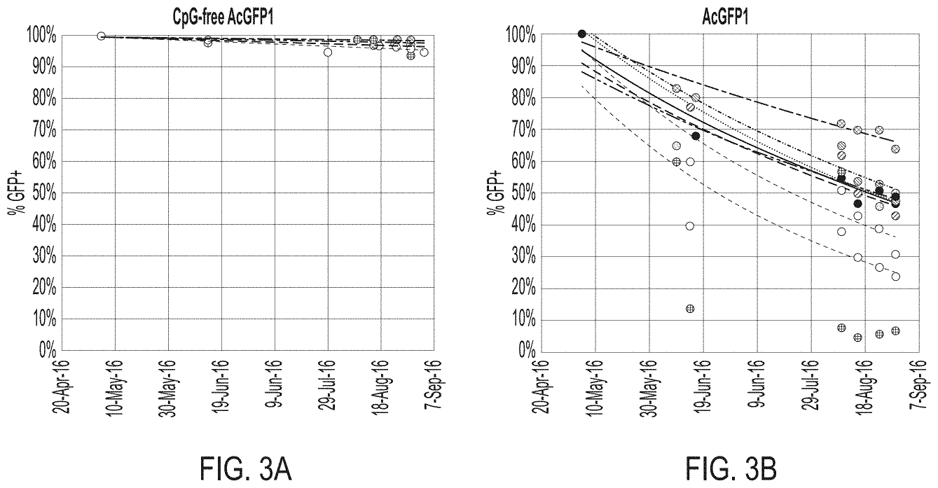

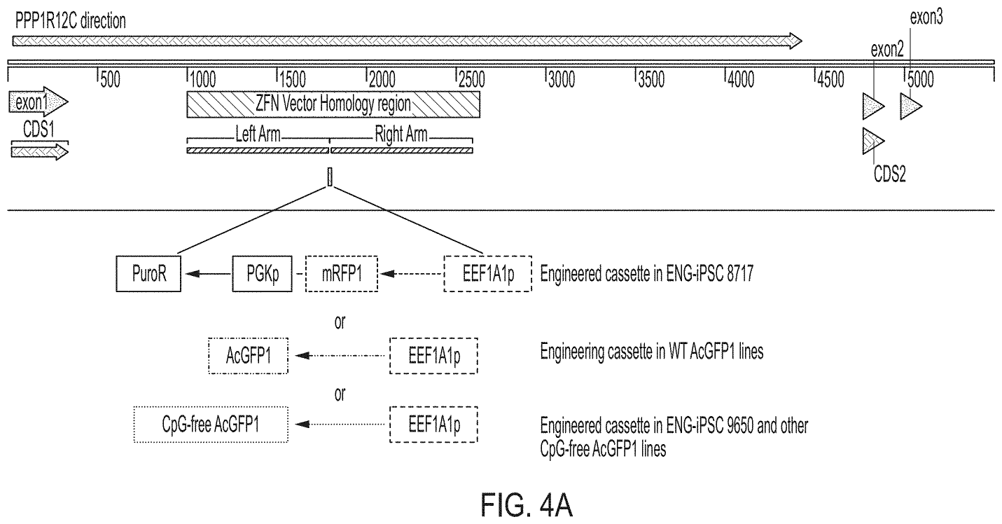

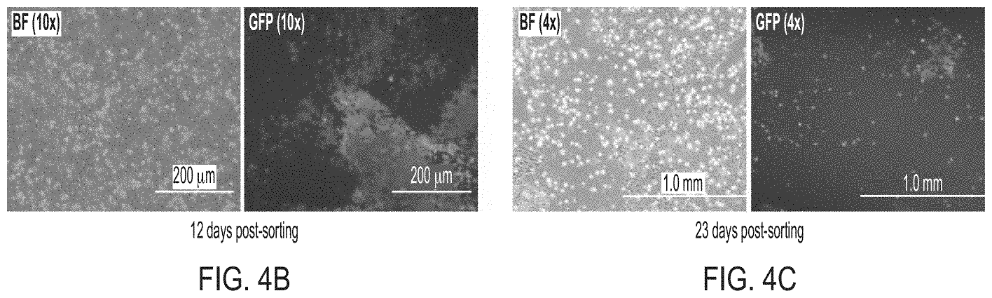

Thus, in certain embodiments, the present disclosure provides methods for maintaining expression of a transgene in a cell line by optimizing the sequence of the transgene to remove CpG motifs and, thus, prevent rapid silencing of the transgene. Methylation is a major epigenetic mechanism in addition to RNA-associate silencing and histone modification. In the present studies, the DNA sequence of Aequorea coerulescens green fluorescence protein (AcGFP1) was modified to remove CpG motifs as depicted in FIG. 2. The results showed that expression of the CpG-free AcGFP1 was stable while the expression of wild-type AcGFP1 was not (FIG. 3). Thus, the present methods allow for the prevention of transgene silencing due to global methylation or other epigenetic dysregulation.

In further embodiments, methods are provided for maintaining expression of a transgene in a cell line by driving expression of the transgene by novel promoters (e.g., SEQ ID NOs: 1-12 or 17) provided herein or by driving expression of the transgene by tagging genes, such as HSP90AB1, ACTB, CTNNB1, or MYL6.

Further, the present cells lines may be differentiated to specific cell types and maintain expression of the transgene for 3 months, 6 months, or even greater than 12 months. In particular aspects, the cell line is cultured for at least 30 days, such as at least 2 months, 3 months, 4 months, 5 months or longer. In particular aspects, the cell line is cultured for over six months, such as over one year, over two years, or over three years. In particular aspects, the cell line has stable expression of the transgene for at least 30 days, such as at least 2 months, 3 months, 4 months, 5 months or longer. In particular aspects, the cell line has stable expression of the transgene over six months, such as over one year, over two years, or over three years. In additional aspects, methods are provided for the cellular assays for use of the present cell lines for cell viability and screening assays.

### I. DEFINITIONS

The term “purified” does not require absolute purity; rather, it is intended as a relative term. Thus, a purified population of cells is greater than about 90% 91%, 92%, 93%, 94%, 95%, 96%, 97%, 98%, 99% or 100% pure, or, most preferably, essentially free of other cell types.

As used herein, the term “stable expression” refers to expression that is more stable than the unmodified sequence. For example, stable expression may refer to expression that remains unchanged over a period of time, such as one month, six months, a year, or greater than a year.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

As used herein in the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

The term “essentially” is to be understood that methods or compositions include only the specified steps or materials and those that do not materially affect the basic and novel characteristics of those methods and compositions.

The term “substantially free of” is used to 98% of the listed components and less than 2% of the components to which composition or particle is substantially free of.

The terms “substantially” or “approximately” as used herein may be applied to modify any quantitative comparison, value, measurement, or other representation that could permissibly vary without resulting in a change in the basic function to which it is related.

The term “about” means, in general, within a standard deviation of the stated value as determined using a standard analytical technique for measuring the stated value. The terms can also be used by referring to plus or minus 5% of the stated value.

As used herein, a sequence that is “substantially” similar to a wild-type sequence comprises a percent GC content within 5% of the wildtype percent GC content.

The term “cell population” is used herein to refer to a group of cells, typically of a common type. The cell population can be derived from a common progenitor or may comprise more than one cell type. An “enriched” cell population refers to a cell population derived from a starting cell population (e.g., an unfractionated, heterogeneous cell population) that contains a greater percentage of a specific cell type than the percentage of that cell type in the starting population. The cell populations may be enriched for one or more cell types and depleted of one or more cell types.

The term “stem cell” refers herein to a cell that under suitable conditions is capable of differentiating into a diverse range of specialized cell types, while under other suitable conditions is capable of self-renewing and remaining in an essentially undifferentiated pluripotent state. The term “stem cell” also encompasses a pluripotent cell, multipotent cell, precursor cell and progenitor cell. Exemplary human stem cells can be obtained from hematopoietic or mesenchymal stem cells obtained from bone marrow tissue, embryonic stem cells obtained from embryonic tissue, or embryonic germ cells obtained from genital tissue of a fetus. Exemplary pluripotent stem cells can also be produced from somatic cells by reprogramming them to a pluripotent state by the expression of certain transcription factors associated with pluripotency; these cells are called “induced pluripotent stem cells” or “iPSCs”.

The term “pluripotent” refers to the property of a cell to differentiate into all other cell types in an organism, with the exception of extraembryonic, or placental, cells. Pluripotent stem cells are capable of differentiating to cell types of all three germ layers (e.g., ectodermal, mesodermal, and endodermal cell types) even after prolonged culture. A pluripotent stem cell may be an embryonic stem cell derived from the inner cell mass of a blastocyst or produced by nuclear transfer. In other embodiments, the pluripotent stem cell is an induced pluripotent stem cell derived by reprogramming somatic cells.

The term “differentiation” refers to the process by which an unspecialized cell becomes a more specialized type with changes in structural and/or functional properties. The mature cell typically has altered cellular structure and tissue-specific proteins.

As used herein, “undifferentiated” refers to cells that display characteristic markers and morphological characteristics of undifferentiated cells that clearly distinguish them from terminally differentiated cells of embryo or adult origin.

“Embryoid bodies (EBs)” are aggregates of pluripotent stem cells that can undergo differentiation into cells of the endoderm, mesoderm, and ectoderm germ layers. The spheroid structures form when pluripotent stem cells are allowed to aggregate under non-adherent culture conditions and thus form EBs in suspension.

An “isolated” cell has been substantially separated or purified from others cells in an organism or culture. Isolated cells can be, for example, at least 99%, at least 98% pure, at least 95% pure or at least 90% pure.

A “cell line” as used herein refers to a collection of cells originating from one cell. The cell line may be kept in a growth medium in tubes, flasks, or dishes. The cell line may be developed by clonal expansion from a single cell that is allowed to expand to multiple cells. The cell line may comprise cells that are genetically identical and can be maintained in culture over time, such as several months or years.

An “embryo” refers to a cellular mass obtained by one or more divisions of a zygote or an activated oocyte with an artificially reprogrammed nucleus.

An “embryonic stem (ES) cell” is an undifferentiated pluripotent cell which is obtained from an embryo in an early stage, such as the inner cell mass at the blastocyst stage, or produced by artificial means (e.g. nuclear transfer) and can give rise to any differentiated cell type in an embryo or an adult, including germ cells (e.g. sperm and eggs).

“Induced pluripotent stem cells (iPSCs)” are cells generated by reprogramming a somatic cell by expressing or inducing expression of a combination of factors (herein referred to as reprogramming factors). iPSCs can be generated using fetal, postnatal, newborn, juvenile, or adult somatic cells. In certain embodiments, factors that can be used to reprogram somatic cells to pluripotent stem cells include, for example, Oct4 (sometimes referred to as Oct 3/4), Sox2, c-Myc, and Klf4, Nanog, and Lin28. In some embodiments, somatic cells are reprogrammed by expressing at least two reprogramming factors, at least three reprogramming factors, or four reprogramming factors to reprogram a somatic cell to a pluripotent stem cell.

“Feeder-free” or “feeder-independent” is used herein to refer to a culture supplemented with cytokines and growth factors (e.g., TGFβ, bFGF, LIF) as a replacement for the feeder cell layer. Thus, “feeder-free” or feeder-independent culture systems and media may be used to culture and maintain pluripotent cells in an undifferentiated and proliferative state. In some cases, feeder-free cultures utilize an animal-based matrix (e.g. MATRIGEL™) or are grown on a substrate such as fibronectin, collagen, or vitronectin. These approaches allow human stem cells to remain in an essentially undifferentiated state without the need for mouse fibroblast “feeder layers.”

“Feeder layers” are defined herein as a coating layer of cells such as on the bottom of a culture dish. The feeder cells can release nutrients into the culture medium and provide a surface to which other cells, such as pluripotent stem cells, can attach.

The term “defined” or “fully-defined,” when used in relation to a medium, an extracellular matrix, or a culture condition, refers to a medium, an extracellular matrix, or a culture condition in which the chemical composition and amounts of approximately all the components are known. For example, a defined medium does not contain undefined factors such as in fetal bovine serum, bovine serum albumin or human serum albumin. Generally, a defined medium comprises a basal media (e.g., Dulbecco's Modified Eagle's Medium (DMEM), F12, or Roswell Park Memorial Institute Medium (RPMI) 1640, containing amino acids, vitamins, inorganic salts, buffers, antioxidants, and energy sources) which is supplemented with recombinant albumin, chemically defined lipids, and recombinant insulin. An example of a fully defined medium is Essential 8™ medium.

For a medium, extracellular matrix, or culture system used with human cells, the term “Xeno-Free (XF)” refers to a condition in which the materials used are not of non-human animal-origin.

### II. ENGINEERED CELL LINES

In some embodiments, cell lines are provided herein which are engineered to express a transgene with stable expression. The stable expression can be achieved by codon optimizing the transgene sequence to remove CpG motifs, driving expression by novel promoters (e.g., SEQ ID NOs:1-12 or 17), or by driving expression by tagging endogenous gene (e.g., HSP90AB1, ACTB, CTNNB1, or MYL6).

As used herein, “CpG motif” refers to nucleotides contains a cytosine “C” followed by a phosphate bond “p” and a guanine “G”. Reference to “removal of CpG motifs” means that the C and/or G nucleotides are modified to remove the motif. As used herein, “humanized” with respect to a nucleic acid molecule means that the nucleic acid molecule has a sequence or a portion of a sequence that resembles or closely resembles a human sequence or the molecule is otherwise made to be more functional in a human cell. For example, codons can be optimized for human usage based on known codon usage in humans in order to enhance the effectiveness of expression of the nucleic acid in human cells, e.g. to achieve faster translation rates and high accuracy.

TABLE 1
Exemplary replacement codons

| | CpG-free | Amino acid |
|---|---|---|
| WT | modified | sequences |
| codon(s) | codon(s) | (WT/CpG-free) |
| ACG | ACA | T/T |
| | | Thr/Thr |
| AGC GTC GCC | TCT GTG GCC | SVA/SVA |
| | | Ser-Val-Ala/ |
| | | Ser-Val-Ala |
| CGC | AGA | R/R |
| | | Arg/Arg |
| AGC GAC GGC | TCT GAT GGC | SDG/SDG |
| | | Ser-Asp-Gly/ |
| | | Ser-Asp-Gly |
| CTC GTG | CTG GTG | LV/LV |
| | | Leu-Val/ |
| | | Leu-Val |
| GCG | GCA | A/A |
| | | Ala/Ala |
| ATC GTC GCG | ATT GTG GCA | IVA/IVA |
| | | Ile-Val-Ala/ |
| | | Ile-Val-Ala |
| CGA | AGG | R/R |
| | | Arg/Arg |

The process of gene switching off by methylation is explained by a series of cascades of events that ultimately result in changes in chromatin structure, forming a transcription-weak state. Methylation of 5′-CpG-3′ in a gene binds to a methylated DNA sequence and simultaneously binds to a histone deacetylase (MBD-HDAC) and a transcription inhibitor protein (transcriptional redresser protein). Artificial gene synthesis techniques allow any nucleotide sequence selected from this possibility to be synthesized. wherein the amino acid sequence encoded by the corresponding gene is preferably unchanged. The modified target nucleic acid sequences are generated from long oligonucleotides, for example by stepwise PCR, as described in the examples, or for conventional gene synthesis, a specialized supplier (e.g., Geneart GmbH, Qiagen AG).

In some aspects, all CpGs in a transgene that can be removed within the scope of the genetic code are removed. However, less CpGs, for example 50%, 60%, 75% 80%, 85%, 90%, 95%, 96%, 97%, 98%, or 99%, may also be removed. Codon-optimized constructs according to the present disclosure can be prepared, for example, by selecting the same codon distribution is as in the expression system used. The expression system may be a mammalian system, such as a human system. Preferably, the codon optimization thus matches the codon selection of the human gene.

The abundance of some codons in Homo sapiens is low, while other codons are moderate or high. As used herein, a “rare codon” refers to a codon with a frequency of less than 0.2 in Homo sapiens. To avoid the use of rare codons when modifying the DNA sequence, a codon frequency table may be used to select codons with a frequency of at least 0.3, such as at least 0.4, 0.5, 0.6, 0.7, 0.8, or 0.9.

TABLE 2
Codon frequency in homo sapiens.

| | | | | | | | | | | | | | | | | | | | |
|---|---|---|---|---|---|---|---|---|---|---|---|---|---|---|---|---|---|---|---|
| U | U | U | F | 0.46 | U | C | U | S | 0.19 | U | A | U | Y | 0.44 | U | G | U | C | 0.46 |
| U | U | C | F | 0.54 | U | C | C | S | 0.22 | U | A | C | Y | 0.56 | U | G | C | C | 0.54 |
| U | U | A | L | 0.08 | U | C | A | S | 0.15 | U | A | A | * | 0.30 | U | G | A | * | 0.47 |
| U | U | G | L | 0.13 | U | C | G | S | 0.05 | U | A | G | * | 0.24 | U | G | G | W | 1.00 |
| C | U | U | L | 0.13 | C | C | U | P | 0.29 | C | A | U | H | 0.42 | C | G | U | R | 0.08 |
| C | U | C | L | 0.20 | C | C | C | P | 0.32 | C | A | C | H | 0.58 | C | G | C | R | 0.18 |
| C | U | A | L | 0.07 | C | C | A | P | 0.28 | C | A | A | Q | 0.27 | C | G | A | R | 0.11 |
| C | U | G | L | 0.40 | C | C | G | P | 0.11 | C | A | G | Q | 0.73 | C | G | G | R | 0.20 |
| A | U | U | I | 0.36 | A | C | U | T | 0.25 | A | A | U | N | 0.47 | A | G | U | S | 0.15 |
| A | U | C | I | 0.47 | A | C | C | T | 0.36 | A | A | C | N | 0.53 | A | G | C | S | 0.24 |
| A | U | A | I | 0.17 | A | C | A | T | 0.28 | A | A | A | K | 0.43 | A | G | A | R | 0.21 |
| A | U | G | M | 1.00 | A | C | G | T | 0.11 | A | A | G | K | 0.57 | A | G | G | R | 0.21 |
| G | U | U | V | 0.18 | G | C | U | A | 0.27 | G | A | U | D | 0.46 | G | G | U | G | 0.16 |
| G | U | C | V | 0.24 | G | C | C | A | 0.40 | G | A | C | D | 0.54 | G | G | C | G | 0.34 |
| G | U | A | V | 0.12 | G | C | A | A | 0.23 | G | A | A | E | 0.42 | G | G | A | G | 0.25 |
| G | U | G | V | 0.46 | G | C | G | A | 0.11 | G | A | G | E | 0.58 | G | G | G | G | 0.25 |

For example, when modifying the sequence encoding for L, leucine, there are 6 codons to evaluate (listed here as: codon+frequency): UUA 8%, UUG 13%, CUU 13%, CUC 20%, CUA 7%, or CUG 40%. If a leucine codon CUC is followed by a codon that begins with a G, a CG motif is present and the leucine codon modification to CUG is preferred over the other 4 codons to remove the CG motif and avoid using a rare codon.

Modification of codons like CCG for proline to remove the CG motif could be accomplished using the codon CCC, however if the protein region contains several prolines this modification would create a mononucleotide stretch repeat. Thus, other codons, such as CCU or CCA, could be used for proline to avoid a mononucleotide stretch. As used herein, a “mononucleotide stretch” refers to a region of at least six of the same nucleotide in a row, such as CCCCCC.

The transgene sequence may encode RNA, a derivative or mimetic, peptide, or polypeptide, modified peptide or polypeptide, protein or modified protein thereof. The transgene may also be a chimeric and/or assembled sequence of different wild type sequences, e.g., may encode a fusion protein or mosaic-type assembled polygene construct. Transgenes may also comprise synthetic sequences. In this regard, nucleic acid sequences can be modeled synthetically, such as by using computer models.

The transgenes to be expressed may be the sequence of genes for any protein, for example recombinant protein, artificial polypeptide, fusion protein and equivalents thereof. In some aspects, the transgenes are diagnostic and/or therapeutic peptides, polypeptides or proteins. In some aspects, the transgenes are reporter genes, such as but not limited to, GFP, RFP, luciferase, β-galactosidase, or chloramphenicol acetyltransferase. In some aspects, the transgene is LacZ, mSEAP, or Lucia. Peptides/proteins include, for example, i) human enzymes (e.g., asparaginase, adenosine deaminase, insulin, tPA, coagulation factor, vitamin K epoxide reductase), hormones (e.g., erythro), production of therapeutic proteins such as poietins, follicle-stimulating hormones, estrogens) and other human-derived proteins (e.g., osteogenic proteins, antithrombin), ii) viral proteins, bacterial proteins, which can be used as vaccines, or proteins derived from parasites (e.g., HIV, HBV, HCV, influenza, Borrelia, Haemophilus, Meningococcus, Anthrax, Botulin Toxin, Diphtheria Toxin, Tetanus Toxin, Plasmodium, etc.) or iii) diagnostics. The transgene may be a promoter or selection gene, such as blasticidin or neomycin.

A. Induced Pluripotent Stem Cells

In some embodiments, the engineered cell lines are iPSCs. The induction of pluripotency was originally achieved in 2006 using mouse cells (Yamanaka et al. 2006) and in 2007 using human cells (Yu et al. 2007; Takahashi et al. 2007) by reprogramming of somatic cells via the introduction of transcription factors that are linked to pluripotency. Pluripotent stem cells can be maintained in an undifferentiated state and can differentiate into any adult cell type.

With the exception of germ cells, any somatic cell can be used as a starting point for iPSCs. For example, cell types could be keratinocytes, fibroblasts, hematopoietic cells, mesenchymal cells, liver cells, or stomach cells. T cells may also be used as a source of somatic cells for reprogramming (U.S. Pat. No. 8,741,648). There is no limitation on the degree of cell differentiation or the age of an animal from which cells are collected; even undifferentiated progenitor cells (including somatic stem cells) and finally differentiated mature cells can be used as sources of somatic cells in the methods disclosed herein. iPSCs can be grown under conditions that are known to differentiate human ES cells into specific cell types, and express human ES cell markers including: SSEA-1, SSEA-3, SSEA-4, TRA-1-60, and TRA-1-81.

A. HLA Matching

Major Histocompatibility Complex (MHC) is the main cause of immune-rejection of allogeneic organ transplants. There are three major class I MHC haplotypes (A, B, and C) and three major MHC class II haplotypes (DR, DP, and DQ).

MHC compatibility between a donor and a recipient increases significantly if the donor cells are HLA homozygous, i.e. contain identical alleles for each antigen-presenting protein. Most individuals are heterozygous for MHC class I and II genes, but certain individuals are homozygous for these genes. These homozygous individuals can serve as super donors, and grafts generated from their cells can be transplanted in all individuals that are either homozygous or heterozygous for that haplotype. Furthermore, if homozygous donor cells have a haplotype found in high frequency in a population, these cells may have application in transplantation therapies for a large number of individuals.

Accordingly, the iPSCs can be produced from somatic cells of the subject to be treated, or another subject with the same or substantially the same HLA type as that of the patient. In one case, the major HLAs (e.g., the three major loci of HLA-A, HLA-B and HLA-DR) of the donor are identical to the major HLAs of the recipient. In some cases, the somatic cell donor may be a super donor; thus, iPSCs derived from a MHC homozygous super donor may be used to generate differentiated cells. Thus, the iPSCs derived from a super donor may be transplanted in subjects that are either homozygous or heterozygous for that haplotype. For example, the iPSCs can be homozygous at two HLA alleles such as HLA-A and HLA-B. As such, iPSCs produced from super donors can be used in the methods disclosed herein, to produce differentiated cells that can potentially “match” a large number of potential recipients.

B. Reprogramming Factors

Somatic cells can be reprogrammed to produce induced pluripotent stem cells (iPSCs) using methods known to one of skill in the art. One of skill in the art can readily produce induced pluripotent stem cells; see for example, Published U.S. Patent Application No. 20090246875, Published U.S. Patent Application No. 2010/0210014; Published U.S. Patent Application No. 20120276636; U.S. Pat. Nos. 8,058,065; 8,129,187; 8,278,620; PCT Publication NO. WO 2007/069666 A1, and U.S. Pat. No. 8,268,620, which are incorporated herein by reference. Generally, nuclear reprogramming factors are used to produce pluripotent stem cells from a somatic cell. In some embodiments, at least two, at least three, or at least four, of Klf4, c-Myc, Oct3/4, Sox2, Nanog, and Lin28 are utilized. In other embodiments, Oct3/4, Sox2, c-Myc and Klf4 are utilized. In some aspects, five, six, seven, or eight reprogramming factors are used.

The cells are treated with a nuclear reprogramming substance, which is generally one or more factor(s) capable of inducing an iPSC from a somatic cell or a nucleic acid that encodes these substances (including forms integrated in a vector). The nuclear reprogramming substances generally include at least Oct3/4, Klf4 and Sox2 or nucleic acids that encode these molecules. A functional inhibitor of p53, L-myc or a nucleic acid that encodes L-myc, and Lin28 or Lin28b or a nucleic acid that encodes Lin28 or Lin28b, can be utilized as additional nuclear reprogramming substances. Nanog can also be utilized for nuclear reprogramming. As disclosed in published U.S. Patent Application No. 20120196360, exemplary reprogramming factors for the production of iPSCs include (1) Oct3/4, Klf4, Sox2, L-Myc (Sox2 can be replaced with Sox1, Sox3, Sox15, Sox17 or Sox18; Klf4 is replaceable with Klf1, Klf2 or Klf5); (2) Oct3/4, Klf4, Sox2, L-Myc, TERT, SV40 Large T antigen (SV40LT); (3) Oct3/4, Klf4, Sox2, L-Myc, TERT, human papilloma virus (HPV)16 E6; (4) Oct3/4, Klf4, Sox2, L-Myc, TERT, HPV16 E7 (5) Oct3/4, Klf4, Sox2, L-Myc, TERT, HPV16 E6, HPV16 E7; (6) Oct3/4, Klf4, Sox2, L-Myc, TERT, Bmil; (7) Oct3/4, Klf4, Sox2, L-Myc, Lin28; (8) Oct3/4, Klf4, Sox2, L-Myc, Lin28, SV40LT; (9) Oct3/4, Klf4, Sox2, L-Myc, Lin28, TERT, SV40LT; (10) Oct3/4, Klf4, Sox2, L-Myc, SV40LT; (11) Oct3/4, Esrrb, Sox2, L-Myc (Esrrb is replaceable with Esrrg); (12) Oct3/4, Klf4, Sox2; (13) Oct3/4, Klf4, Sox2, TERT, SV40LT; (14) Oct3/4, Klf4, Sox2, TERT, HP VI 6 E6; (15) Oct3/4, Klf4, Sox2, TERT, HPV16 E7; (16) Oct3/4, Klf4, Sox2, TERT, HPV16 E6, HPV16 E7; (17) Oct3/4, Klf4, Sox2, TERT, Bmil; (18) Oct3/4, Klf4, Sox2, Lin28 (19) Oct3/4, Klf4, Sox2, Lin28, SV40LT; (20) Oct3/4, Klf4, Sox2, Lin28, TERT, SV40LT; (21) Oct3/4, Klf4, Sox2, SV40LT; or (22) Oct3/4, Esrrb, Sox2 (Esrrb is replaceable with Esrrg). In one non-limiting example, Oct3/4, Klf4, Sox2, and c-Myc are utilized. In other embodiments, Oct4, Nanog, and Sox2 are utilized; see for example, U.S. Pat. No. 7,682,828, which is incorporated herein by reference. These factors include, but are not limited to, Oct3/4, Klf4 and Sox2. In other examples, the factors include, but are not limited to Oct 3/4, Klf4 and Myc. In some non-limiting examples, Oct3/4, Klf4, c-Myc, and Sox2 are utilized. In other non-limiting examples, Oct3/4, Klf4, Sox2 and Sal 4 are utilized. Factors like Nanog, Lin28, Klf4, or c-Myc can increase reprogramming efficiency and can be expressed from several different expression vectors. For example, an integrating vector such as the EBV element-based system can be used (U.S. Pat. No. 8,546,140). In a further aspect, reprogramming proteins could be introduced directly into somatic cells by protein transduction. Reprogramming may further comprise contacting the cells with one or more signaling receptors including glycogen synthase kinase 3 (GSK-3) inhibitor, a mitogen-activated protein kinase kinase (MEK) inhibitor, a transforming growth factor beta (TGF-β) receptor inhibitor or signaling inhibitor, leukemia inhibitory factor (LIF), a p53 inhibitor, an NF-kappa B inhibitor, or a combination thereof. Those regulators may include small molecules, inhibitory nucleotides, expression cassettes, or protein factors. It is anticipated that virtually any iPS cells or cell lines may be used.

Mouse and human cDNA sequences of these nuclear reprogramming substances are available with reference to the NCBI accession numbers mentioned in WO 2007/069666, which is incorporated herein by reference. Methods for introducing one or more reprogramming substances, or nucleic acids encoding these reprogramming substances, are known in the art, and disclosed for example, in published U.S. Patent Application No. 2012/0196360 and U.S. Pat. No. 8,071,369, which both are incorporated herein by reference.

Once derived, iPSCs can be cultured in a medium sufficient to maintain pluripotency. The iPSCs may be used with various media and techniques developed to culture pluripotent stem cells, more specifically, embryonic stem cells, as described in U.S. Pat. No. 7,442,548 and U.S. Patent Pub. No. 2003/0211603. In the case of mouse cells, the culture is carried out with the addition of Leukemia Inhibitory Factor (LIF) as a differentiation suppression factor to an ordinary medium. In the case of human cells, it is desirable that basic fibroblast growth factor (bFGF) be added in place of LIF. Other methods for the culture and maintenance of iPSCs, as would be known to one of skill in the art, may be used.

In certain embodiments, undefined conditions may be used; for example, pluripotent cells may be cultured on fibroblast feeder cells or a medium that has been exposed to fibroblast feeder cells in order to maintain the stem cells in an undifferentiated state. In some embodiments, the cell is cultured in the co-presence of mouse embryonic fibroblasts treated with radiation or an antibiotic to terminate the cell division, as feeder cells. Alternately, pluripotent cells may be cultured and maintained in an essentially undifferentiated state using a defined, feeder-independent culture system, such as a TESR™ medium (Ludwig et al., 2006a; Ludwig et al., 2006b) or E8™ medium (Chen et al., 2011).

C. Plasmids

In some embodiments, the iPSC can be modified to express exogenous nucleic acids, such as to include an enhancer operably linked to a promoter and a nucleic acid sequence encoding a first marker. The construct can also include other elements, such as a ribosome binding site for translational initiation (internal ribosomal binding sequences), and a transcription/translation terminator. Generally, it is advantageous to transfect cells with the construct. Suitable vectors for stable transfection include, but are not limited to retroviral vectors, lentiviral vectors and Sendai virus.

In some embodiments plasmids that encode a marker are composed of: (1) a high copy number replication origin, (2) a selectable marker, such as, but not limited to, the neo gene for antibiotic selection with kanamycin, (3) transcription termination sequences, including the tyrosinase enhancer and (4) a multicloning site for incorporation of various nucleic acid cassettes; and (5) a nucleic acid sequence encoding a marker operably linked to the tyrosinase promoter. There are numerous plasmid vectors that are known in the art for inducing a nucleic acid encoding a protein. These include, but are not limited to, the vectors disclosed in U.S. Pat. Nos. 6,103,470; 7,598,364; 7,989,425; and 6,416,998, which are incorporated herein by reference. In some aspects, the plasmid comprises a “suicide gene” which, upon administration of a prodrug or drug, effects transition of a gene product to a compound which kills its host cell. Examples of suicide gene, prodrug or drug combinations which may be used are, for example, without limiting, truncated EGFR and cetuximab; Herpes Simplex Virus-thymidine kinase (HSV-tk) and ganciclovir, acyclovir, or FIAU; oxidoreductase and cycloheximide; cytosine deaminase and 5-fluorocytosine; thymidine kinase thymidilate kinase (Tdk::Tmk) and AZT; and deoxycytidine kinase and cytosine arabinoside.

A viral gene delivery system can be an RNA-based or DNA-based viral vector. An episomal gene delivery system can be a plasmid, an Epstein-Barr virus (EBV)-based episomal vector, a yeast-based vector, an adenovirus-based vector, a simian virus 40 (SV40)-based episomal vector, a bovine papilloma virus (BPV)-based vector, or a lentiviral vector.

Markers include, but are not limited to, fluorescence proteins (for example, green fluorescent protein or red fluorescent protein), enzymes (for example, horse radish peroxidase or alkaline phosphatase or firefly/renilla luciferase or nanoluc), or other proteins. A marker may be a protein (including secreted, cell surface, or internal proteins; either synthesized or taken up by the cell); a nucleic acid (such as an mRNA, or enzymatically active nucleic acid molecule) or a polysaccharide. Included are determinants of any such cell components that are detectable by antibody, lectin, probe or nucleic acid amplification reaction that are specific for the marker of the cell type of interest. The markers can also be identified by a biochemical or enzyme assay or biological response that depends on the function of the gene product. Nucleic acid sequences encoding these markers can be operably linked to the tyrosinase enhancer. In addition, other genes can be included, such as genes that may influence stem cell differentiation, or cell function, or physiology, or pathology.

D. Delivery Systems

Introduction of a nucleic acid, such as DNA or RNA, into the engineered cells lines of the current disclosure may use any suitable methods for nucleic acid delivery for transformation of a cell, as described herein or as would be known to one of ordinary skill in the art. Such methods include, but are not limited to, direct delivery of DNA such as by ex vivo transfection (Wilson et al., 1989, Nabel et al, 1989), by injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harland and Weintraub, 1985; U.S. Pat. No. 5,789,215, incorporated herein by reference); by electroporation (U.S. Pat. No. 5,384,253, incorporated herein by reference; Tur-Kaspa et al., 1986; Potter et al., 1984); by calcium phosphate precipitation (Graham and Van Der Eb, 1973; Chen and Okayama, 1987; Rippe et al., 1990); by using DEAE-dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991) and receptor-mediated transfection (Wu and Wu, 1987; Wu and Wu, 1988); by microprojectile bombardment (PCT Application Nos. WO 94/09699 and 95/06128; U.S. Pat. Nos. 5,610,042; 5,322,783 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); by Agrobacterium-mediated transformation (U.S. Pat. Nos. 5,591,616 and 5,563,055, each incorporated herein by reference); by desiccation/inhibition-mediated DNA uptake (Potrykus et al., 1985), and any combination of such methods. Through the application of techniques such as these, organelle(s), cell(s), tissue(s) or organism(s) may be stably or transiently transformed.

1. Viral Vectors

Viral vectors may be provided in certain aspects of the present disclosure. In generating recombinant viral vectors, non-essential genes are typically replaced with a gene or coding sequence for a heterologous (or non-native) protein. A viral vector is a kind of expression construct that utilizes viral sequences to introduce nucleic acid and possibly proteins into a cell. The ability of certain viruses to infect cells or enter cells via receptor-mediated endocytosis, and to integrate into host cell genomes and express viral genes stably and efficiently have made them attractive candidates for the transfer of foreign nucleic acids into cells (e.g., mammalian cells). Non-limiting examples of virus vectors that may be used to deliver a nucleic acid of certain aspects of the present disclosure are described below.

Retroviruses have promise as gene delivery vectors due to their ability to integrate their genes into the host genome, transfer a large amount of foreign genetic material, infect a broad spectrum of species and cell types, and be packaged in special cell-lines (Miller, 1992).

In order to construct a retroviral vector, a nucleic acid is inserted into the viral genome in place of certain viral sequences to produce a virus that is replication-defective. In order to produce virions, a packaging cell line containing the gag, pol, and env genes—but without the LTR and packaging components—is constructed (Mann et al., 1983). When a recombinant plasmid containing a cDNA, together with the retroviral LTR and packaging sequences, is introduced into a special cell line (e.g., by calcium phosphate precipitation), the packaging sequence allows the RNA transcript of the recombinant plasmid to be packaged into viral particles, which are then secreted into the culture medium (Nicolas and Rubenstein, 1988; Temin, 1986; Mann et al., 1983). The medium containing the recombinant retroviruses is then collected, optionally concentrated, and used for gene transfer. Retroviral vectors are able to infect a broad variety of cell types. However, integration and stable expression require the division of host cells (Paskind et al., 1975).

Lentiviruses are complex retroviruses, which, in addition to the common retroviral genes gag, pol, and env, contain other genes with regulatory or structural function. Lentiviral vectors are well known in the art (see, for example, Naldini et al., 1996; Zufferey et al., 1997; Blomer et al., 1997; U.S. Pat. Nos. 6,013,516 and 5,994,136).

Recombinant lentiviral vectors are capable of infecting non-dividing cells and can be used for both in vivo and ex vivo gene transfer and expression of nucleic acid sequences. For example, recombinant lentivirus capable of infecting a non-dividing cell—wherein a suitable host cell is transfected with two or more vectors carrying the packaging functions, namely gag, pol and env, as well as rev and tat—is described in U.S. Pat. No. 5,994,136, incorporated herein by reference.

2. Episomal Vectors

The use of plasmid- or liposome-based extra-chromosomal (i.e., episomal) vectors may be also provided in certain aspects of the present disclosure. Such episomal vectors may include, e.g., oriP-based vectors, and/or vectors encoding a derivative of EBNA-1. These vectors may permit large fragments of DNA to be introduced unto a cell and maintained extra-chromosomally, replicated once per cell cycle, partitioned to daughter cells efficiently, and elicit substantially no immune response.

In particular, EBNA-1, the only viral protein required for the replication of the oriP-based expression vector, does not elicit a cellular immune response because it has developed an efficient mechanism to bypass the processing required for presentation of its antigens on MHC class I molecules (Levitskaya et al., 1997). Further, EBNA-1 can act in trans to enhance expression of the cloned gene, inducing expression of a cloned gene up to 100-fold in some cell lines (Langle-Rouault et al., 1998; Evans et al., 1997). Finally, the manufacture of such oriP-based expression vectors is inexpensive.

In certain aspects, reprogramming factors are expressed from expression cassettes comprised in one or more exogenous episiomal genetic elements (see U.S. Patent Publication 2010/0003757, incorporated herein by reference). Thus, iPSCs can be essentially free of exogenous genetic elements, such as from retroviral or lentiviral vector elements. These iPSCs are prepared by the use of extra-chromosomally replicating vectors (i.e., episomal vectors), which are vectors capable of replicating episomally to make iPSCs essentially free of exogenous vector or viral elements (see U.S. Pat. No. 8,546,140, incorporated herein by reference; Yu et al., 2009). A number of DNA viruses, such as adenoviruses, Simian vacuolating virus 40 (SV40) or bovine papilloma virus (BPV), or budding yeast ARS (Autonomously Replicating Sequences)—containing plasmids replicate extra-chromosomally or episomally in mammalian cells. These episomal plasmids are intrinsically free from all these disadvantages (Bode et al., 2001) associated with integrating vectors. For example, a lymphotrophic herpes virus-based including or Epstein-Barr Virus (EBV) as defined above may replicate extra-chromosomally and help deliver reprogramming genes to somatic cells. Useful EBV elements are OriP and EBNA-1, or their variants or functional equivalents. An additional advantage of episomal vectors is that the exogenous elements will be lost with time after being introduced into cells, leading to self-sustained iPSCs essentially free of these elements.

Other extra-chromosomal vectors include other lymphotrophic herpes virus-based vectors. Lymphotrophic herpes virus is a herpes virus that replicates in a lymphoblast (e.g., a human B lymphoblast) and becomes a plasmid for a part of its natural life-cycle. Herpes simplex virus (HSV) is not a “lymphotrophic” herpes virus. Exemplary lymphotrophic herpes viruses include, but are not limited to EBV, Kaposi's sarcoma herpes virus (KSHV); Herpes virus saimiri (HS) and Marek's disease virus (MDV). Also, other sources of episome-based vectors are contemplated, such as yeast ARS, adenovirus, SV40, or BPV.

One of skill in the art would be well-equipped to construct a vector through standard recombinant techniques (see, for example, Maniatis et al., 1988 and Ausubel et al., 1994, both incorporated herein by reference).

Vectors can also comprise other components or functionalities that further modulate gene delivery and/or gene expression, or that otherwise provide beneficial properties to the targeted cells. Such other components include, for example, components that influence binding or targeting to cells (including components that mediate cell-type or tissue-specific binding); components that influence uptake of the vector nucleic acid by the cell; components that influence localization of the polynucleotide within the cell after uptake (such as agents mediating nuclear localization); and components that influence expression of the polynucleotide.

Such components also may include markers, such as detectable and/or selection markers that can be used to detect or select for cells that have taken up and are expressing the nucleic acid delivered by the vector. Such components can be provided as a natural feature of the vector (such as the use of certain viral vectors that have components or functionalities mediating binding and uptake), or vectors can be modified to provide such functionalities. A large variety of such vectors are known in the art and are generally available. When a vector is maintained in a host cell, the vector can either be stably replicated by the cells during mitosis as an autonomous structure, incorporated within the genome of the host cell, or maintained in the host cell's nucleus or cytoplasm.

3. Regulatory Elements

Expression cassettes included in reprogramming vectors useful in the present disclosure preferably contain (in a 5′-to-3′ direction) a eukaryotic transcriptional promoter operably linked to a protein-coding sequence, splice signals including intervening sequences, and a transcriptional termination/polyadenylation sequence.

a. Promoter/Enhancers

The expression constructs provided herein comprise promoter to drive expression of the programming genes. A promoter generally comprises a sequence that functions to position the start site for RNA synthesis. The best known example of this is the TATA box, but in some promoters lacking a TATA box, such as, for example, the promoter for the mammalian terminal deoxynucleotidyl transferase gene and the promoter for the SV40 late genes, a discrete element overlying the start site itself helps to fix the place of initiation. Additional promoter elements regulate the frequency of transcriptional initiation. Typically, these are located in the region 30-110 bp upstream of the start site, although a number of promoters have been shown to contain functional elements downstream of the start site as well. To bring a coding sequence “under the control of” a promoter, one positions the 5′ end of the transcription initiation site of the transcriptional reading frame “downstream” of (i.e., 3′ of) the chosen promoter. The “upstream” promoter stimulates transcription of the DNA and promotes expression of the encoded RNA.

The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either cooperatively or independently to activate transcription. A promoter may or may not be used in conjunction with an “enhancer,” which refers to a cis-acting regulatory sequence involved in the transcriptional activation of a nucleic acid sequence.

A promoter may be one naturally associated with a nucleic acid sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other virus, or prokaryotic or eukaryotic cell, and promoters or enhancers not “naturally occurring,” i.e., containing different elements of different transcriptional regulatory regions, and/or mutations that alter expression. For example, promoters that are most commonly used in recombinant DNA construction include the β-lactamase (penicillinase), lactose and tryptophan (trp) promoter systems. In addition to producing nucleic acid sequences of promoters and enhancers synthetically, sequences may be produced using recombinant cloning and/or nucleic acid amplification technology, including PCR™, in connection with the compositions disclosed herein (see U.S. Pat. Nos. 4,683,202 and 5,928,906, each incorporated herein by reference). Furthermore, it is contemplated that the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the organelle, cell type, tissue, organ, or organism chosen for expression. Those of skill in the art of molecular biology generally know the use of promoters, enhancers, and cell type combinations for protein expression, (see, for example Sambrook et al. 1989, incorporated herein by reference). The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

Additionally any promoter/enhancer combination (as per, for example, the Eukaryotic Promoter Data Base EPDB) could also be used to drive expression. Use of a T3, T7 or SP6 cytoplasmic expression system is another possible embodiment. Eukaryotic cells can support cytoplasmic transcription from certain bacterial promoters if the appropriate bacterial polymerase is provided, either as part of the delivery complex or as an additional genetic expression construct.

Non-limiting examples of promoters include early or late viral promoters, such as, SV40 early or late promoters, cytomegalovirus (CMV) immediate early promoters, Rous Sarcoma Virus (RSV) early promoters; eukaryotic cell promoters, such as, e. g., beta actin promoter (Ng, 1989; Quitsche et al., 1989), GADPH promoter (Alexander et al., 1988, Ercolani et al., 1988), metallothionein promoter (Karin et al., 1989; Richards et al., 1984); and concatenated response element promoters, such as cyclic AMP response element promoters (cre), serum response element promoter (sre), phorbol ester promoter (TPA) and response element promoters (tre) near a minimal TATA box. It is also possible to use human growth hormone promoter sequences (e.g., the human growth hormone minimal promoter described at Genbank, accession no. X05244, nucleotide 283-341) or a mouse mammary tumor promoter (available from the ATCC, Cat. No. ATCC 45007).

Tissue-specific transgene expression, especially for reporter gene expression in hematopoietic cells and precursors of hematopoietic cells derived from programming, may be desirable as a way to identify derived hematopoietic cells and precursors. To increase both specificity and activity, the use of cis-acting regulatory elements has been contemplated. For example, a hematopoietic cell-specific promoter may be used. Many such hematopoietic cell-specific promoters are known in the art.

In certain aspects, methods of the present disclosure also concern enhancer sequences, i.e., nucleic acid sequences that increase a promoter's activity and that have the potential to act in cis, and regardless of their orientation, even over relatively long distances (up to several kilobases away from the target promoter). However, enhancer function is not necessarily restricted to such long distances as they may also function in close proximity to a given promoter.

Many hematopoietic cell promoter and enhancer sequences have been identified, and may be useful in present methods. See, e.g., U.S. Pat. No. 5,556,954; U.S. Patent App. 20020055144; U.S. Patent App. 20090148425.

b. Initiation Signals and Linked Expression

A specific initiation signal also may be used in the expression constructs provided in the present disclosure for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

In certain embodiments, internal ribosome entry sites (IRES) elements are used to create multigene, or polycistronic, messages. IRES elements are able to bypass the ribosome scanning model of 5′ methylated Cap dependent translation and begin translation at internal sites (Pelletier and Sonenberg, 1988). IRES elements from two members of the picornavirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message (see U.S. Pat. Nos. 5,925,565 and 5,935,819, each herein incorporated by reference).

Additionally, certain 2A sequence elements could be used to create linked-or co-expression of programming genes in the constructs provided in the present disclosure. For example, cleavage sequences could be used to co-express genes by linking open reading frames to form a single cistron. An exemplary cleavage sequence is the F2A (Foot-and-mouth disease virus 2A) or a “2A-like” sequence (e.g., Thosea asigna virus 2A; T2A) (Minskaia and Ryan, 2013). In particular embodiments, an F2A-cleavage peptide is used to link expression of the genes in the multi-lineage construct.

c. Origins of Replication

In order to propagate a vector in a host cell, it may contain one or more origins of replication sites (often termed “ori”), for example, a nucleic acid sequence corresponding to oriP of EBV as described above or a genetically engineered oriP with a similar or elevated function in programming, which is a specific nucleic acid sequence at which replication is initiated. Alternatively, a replication origin of other extra-chromosomally replicating virus as described above or an autonomously replicating sequence (ARS) can be employed.

d. Selection and Screenable Markers

In certain embodiments, cells containing a nucleic acid construct may be identified in vitro or in vivo by including a marker in the expression vector. Such markers would confer an identifiable change to the cell permitting easy identification of cells containing the expression vector. Generally, a selection marker is one that confers a property that allows for selection. A positive selection marker is one in which the presence of the marker allows for its selection, while a negative selection marker is one in which its presence prevents its selection. An example of a positive selection marker is a drug resistance marker.

Usually the inclusion of a drug selection marker aids in the cloning and identification of transformants, for example, genes that confer resistance to neomycin, puromycin, hygromycin, DHFR, GPT, zeocin and histidinol are useful selection markers. In addition to markers conferring a phenotype that allows for the discrimination of transformants based on the implementation of conditions, other types of markers including screenable markers such as GFP, whose basis is colorimetric analysis, are also contemplated. Alternatively, screenable enzymes as negative selection markers such as herpes simplex virus thymidine kinase (tk) or chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selection and screenable markers are well known to one of skill in the art.

E. Gene Editing

In some embodiments, the present methods comprise gene editing by sequence-specific or targeted nucleases, including DNA-binding targeted nucleases such as zinc finger nucleases (ZFN) and transcription activator-like effector nucleases (TALENs), and RNA-guided nucleases such as a CRISPR-associated nuclease (Cas), specifically designed to be targeted to the sequence of the gene or a portion thereof.

In some embodiments, gene editing is carried out by induction of one or more double-stranded breaks and/or one or more single-stranded breaks in the gene, typically in a targeted manner. In some embodiments, the double-stranded or single-stranded breaks are made by a nuclease, e.g., an endonuclease, such as a gene-targeted nuclease. In some aspects, the breaks are induced in the coding region of the gene, e.g., in an exon. For example, in some embodiments, the induction occurs near the N-terminal portion of the coding region, e.g., in the first exon, in the second exon, or in a subsequent exon.

In some aspects, the double-stranded or single-stranded breaks undergo repair via a cellular repair process, such as by non-homologous end-joining (NHEJ) or homology-directed repair (HDR). In some aspects, the repair process is error-prone and results in disruption of the gene, such as a frameshift mutation, e.g., biallelic frameshift mutation, which can result in complete knockout of the gene.

In some embodiments, the gene editing is achieved using a DNA-targeting molecule, such as a DNA-binding protein or DNA-binding nucleic acid, or complex, compound, or composition, containing the same, which specifically binds to or hybridizes to the gene. In some embodiments, the DNA-targeting molecule comprises a DNA-binding domain, e.g., a zinc finger protein (ZFP) DNA-binding domain, a transcription activator-like protein (TAL) or TAL effector (TALE) DNA-binding domain, a clustered regularly interspaced short palindromic repeats (CRISPR) DNA-binding domain, or a DNA-binding domain from a meganuclease. Zinc finger, TALE, and CRISPR system binding domains can be engineered to bind to a predetermined nucleotide sequence, for example via engineering (altering one or more amino acids) of the recognition helix region of a naturally occurring zinc finger or TALE protein. Engineered DNA binding proteins (zinc fingers or TALEs) are proteins that are non-naturally occurring. Rational criteria for design include application of substitution rules and computerized algorithms for processing information in a database storing information of existing ZFP and/or TALE designs and binding data. See, for example, U.S. Pat. Nos. 6,140,081; 6,453,242; and 6,534,261; see also WO 98/53058; WO 98/53059; WO 98/53060; WO 02/016536 and WO 03/016496 and U.S. Publication No. 2011/0301073.

In some embodiments, the DNA-targeting molecule, complex, or combination contains a DNA-binding molecule and one or more additional domain, such as an effector domain to facilitate the repression or disruption of the gene. For example, in some embodiments, the gene editing is carried out by fusion proteins that comprise DNA-binding proteins and a heterologous regulatory domain or functional fragment thereof. In some aspects, domains include, e.g., transcription factor domains such as activators, repressors, co-activators, co-repressors, silencers, oncogenes, DNA repair enzymes and their associated factors and modifiers, DNA rearrangement enzymes and their associated factors and modifiers, chromatin associated proteins and their modifiers, e.g. kinases, acetylases and deacetylases, and DNA modifying enzymes, e.g. methyltransferases, topoisomerases, helicases, ligases, kinases, phosphatases, polymerases, endonucleases, and their associated factors and modifiers. See, for example, U.S. Patent Application Publication Nos. 2005/0064474; 2006/0188987 and 2007/0218528, incorporated by reference in their entireties herein, for details regarding fusions of DNA-binding domains and nuclease cleavage domains. In some aspects, the additional domain is a nuclease domain. Thus, in some embodiments, gene editing is facilitated by gene or genome editing, using engineered proteins, such as nucleases and nuclease-containing complexes or fusion proteins, composed of sequence-specific DNA-binding domains fused to or complexed with non-specific DNA-cleavage molecules such as nucleases.

In some aspects, these targeted chimeric nucleases or nuclease-containing complexes carry out precise genetic modifications by inducing targeted double-stranded breaks or single-stranded breaks, stimulating the cellular DNA-repair mechanisms, including error-prone nonhomologous end joining (NHEJ) and homology-directed repair (HDR). In some embodiments the nuclease is an endonuclease, such as a zinc finger nuclease (ZFN), TALE nuclease (TALEN), and RNA-guided endonuclease (RGEN), such as a CRISPR-associated (Cas) protein, or a meganuclease.

In some embodiments, a donor nucleic acid, e.g., a donor plasmid or nucleic acid encoding the genetically engineered antigen receptor, is provided and is inserted by HDR at the site of gene editing following the introduction of the DSBs. Thus, in some embodiments, the disruption of the gene and the introduction of the antigen receptor, e.g., CAR, are carried out simultaneously, whereby the gene is disrupted in part by knock-in or insertion of the CAR-encoding nucleic acid.

In some embodiments, no donor nucleic acid is provided. In some aspects, NHEJ-mediated repair following introduction of DSBs results in insertion or deletion mutations that can cause gene disruption, e.g., by creating missense mutations or frameshifts.

1. ZFPs and ZFNs

In some embodiments, the DNA-targeting molecule includes a DNA-binding protein such as one or more zinc finger protein (ZFP) or transcription activator-like protein (TAL), fused to an effector protein such as an endonuclease. Examples include ZFNs, TALEs, and TALENs.

In some embodiments, the DNA-targeting molecule comprises one or more zinc-finger proteins (ZFPs) or domains thereof that bind to DNA in a sequence-specific manner. A ZFP or domain thereof is a protein or domain within a larger protein that binds DNA in a sequence-specific manner through one or more zinc fingers, regions of amino acid sequence within the binding domain whose structure is stabilized through coordination of a zinc ion. The term zinc finger DNA binding protein is often abbreviated as zinc finger protein or ZFP. Among the ZFPs are artificial ZFP domains targeting specific DNA sequences, typically 9-18 nucleotides long, generated by assembly of individual fingers.

ZFPs include those in which a single finger domain is approximately 30 amino acids in length and contains an alpha helix containing two invariant histidine residues coordinated through zinc with two cysteines of a single beta turn, and having two, three, four, five, or six fingers. Generally, sequence-specificity of a ZFP may be altered by making amino acid substitutions at the four helix positions (−1, 2, 3 and 6) on a zinc finger recognition helix. Thus, in some embodiments, the ZFP or ZFP-containing molecule is non-naturally occurring, e.g., is engineered to bind to a target site of choice.

In some aspects, disruption of MeCP2 is carried out by contacting a first target site in the gene with a first ZFP, thereby disrupting the gene. In some embodiments, the target site in the gene is contacted with a fusion ZFP comprising six fingers and the regulatory domain, thereby inhibiting expression of the gene.

In some embodiments, the step of contacting further comprises contacting a second target site in the gene with a second ZFP. In some aspects, the first and second target sites are adjacent. In some embodiments, the first and second ZFPs are covalently linked. In some aspects, the first ZFP is a fusion protein comprising a regulatory domain or at least two regulatory domains.

In some embodiments, the first and second ZFPs are fusion proteins, each comprising a regulatory domain or each comprising at least two regulatory domains. In some embodiments, the regulatory domain is a transcriptional repressor, a transcriptional activator, an endonuclease, a methyl transferase, a histone acetyltransferase, or a histone deacetylase.

In some embodiments, the ZFP is encoded by a ZFP nucleic acid operably linked to a promoter. In some aspects, the method further comprises the step of first administering the nucleic acid to the cell in a lipid:nucleic acid complex or as naked nucleic acid. In some embodiments, the ZFP is encoded by an expression vector comprising a ZFP nucleic acid operably linked to a promoter. In some embodiments, the ZFP is encoded by a nucleic acid operably linked to an inducible promoter. In some aspects, the ZFP is encoded by a nucleic acid operably linked to a weak promoter.

In some embodiments, the target site is upstream of a transcription initiation site of the gene. In some aspects, the target site is adjacent to a transcription initiation site of the gene. In some aspects, the target site is adjacent to an RNA polymerase pause site downstream of a transcription initiation site of the gene.

In some embodiments, the DNA-targeting molecule is or comprises a zinc-finger DNA binding domain fused to a DNA cleavage domain to form a zinc-finger nuclease (ZFN). In some embodiments, fusion proteins comprise the cleavage domain (or cleavage half-domain) from at least one Type liS restriction enzyme and one or more zinc finger binding domains, which may or may not be engineered. In some embodiments, the cleavage domain is from the Type liS restriction endonuclease Fok I. Fok I generally catalyzes double-stranded cleavage of DNA, at 9 nucleotides from its recognition site on one strand and 13 nucleotides from its recognition site on the other.

In some embodiments, ZFNs target a gene present in the engineered cell. In some aspects, the ZFNs efficiently generate a double strand break (DSB), for example at a predetermined site in the coding region of the gene. Typical regions targeted include exons, regions encoding N terminal regions, first exon, second exon, and promoter or enhancer regions. In some embodiments, transient expression of the ZFNs promotes highly efficient and permanent disruption of the target gene in the engineered cells. In particular, in some embodiments, delivery of the ZFNs results in the permanent disruption of the gene with efficiencies surpassing 50%.

Many gene-specific engineered zinc fingers are available commercially. For example, Sangamo Biosciences (Richmond, Calif., USA) has developed a platform (CompoZr) for zinc-finger construction in partnership with Sigma-Aldrich (St. Louis, Mo., USA), allowing investigators to bypass zinc-finger construction and validation altogether, and provides specifically targeted zinc fingers for thousands of proteins (Gaj et al., Trends in Biotechnology, 2013, 31(7), 397-405). In some embodiments, commercially available zinc fingers are used or are custom designed.

2. TALs, TALEs and TALENs

In some embodiments, the DNA-targeting molecule comprises a naturally occurring or engineered (non-naturally occurring) transcription activator-like protein (TAL) DNA binding domain, such as in a transcription activator-like protein effector (TALE) protein, See, e.g., U.S. Patent Publication No. 2011/0301073, incorporated by reference in its entirety herein.

A TALE DNA binding domain or TALE is a polypeptide comprising one or more TALE repeat domains/units. The repeat domains are involved in binding of the TALE to its cognate target DNA sequence. A single “repeat unit” (also referred to as a “repeat”) is typically 33-35 amino acids in length and exhibits at least some sequence homology with other TALE repeat sequences within a naturally occurring TALE protein. Each TALE repeat unit includes 1 or 2 DNA-binding residues making up the Repeat Variable Diresidue (RVD), typically at positions 12 and/or 13 of the repeat. The natural (canonical) code for DNA recognition of these TALEs has been determined such that an HD sequence at positions 12 and 13 leads to a binding to cytosine (C), NG binds to T, NI to A, NN binds to G or A, and NO binds to T and non-canonical (atypical) RVDs are also known. See, U.S. Patent Publication No. 2011/0301073. In some embodiments, TALEs may be targeted to any gene by design of TAL arrays with specificity to the target DNA sequence. The target sequence generally begins with a thymidine.

In some embodiments, the molecule is a DNA binding endonuclease, such as a TALE nuclease (TALEN). In some aspects the TALEN is a fusion protein comprising a DNA-binding domain derived from a TALE and a nuclease catalytic domain to cleave a nucleic acid target sequence.

In some embodiments, the TALEN recognizes and cleaves the target sequence in the gene. In some aspects, cleavage of the DNA results in double-stranded breaks. In some aspects the breaks stimulate the rate of homologous recombination or non-homologous end joining (NHEJ). Generally, NHEJ is an imperfect repair process that often results in changes to the DNA sequence at the site of the cleavage. In some aspects, repair mechanisms involve rejoining of what remains of the two DNA ends through direct re-ligation (Critchlow and Jackson, 1998) or via the so-called microhomology-mediated end joining. In some embodiments, repair via NHEJ results in small insertions or deletions and can be used to disrupt and thereby repress the gene. In some embodiments, the modification may be a substitution, deletion, or addition of at least one nucleotide. In some aspects, cells in which a cleavage-induced mutagenesis event, i.e. a mutagenesis event consecutive to an NHEJ event, has occurred can be identified and/or selected by well-known methods in the art.

In some embodiments, TALE repeats are assembled to specifically target a gene. A library of TALENs targeting 18,740 human protein-coding genes has been constructed. Custom-designed TALE arrays are commercially available through Cellectis Bioresearch (Paris, France), Transposagen Biopharmaceuticals (Lexington, Ky., USA), and Life Technologies (Grand Island, N.Y., USA).

In some embodiments the TALENs are introduced as trans genes encoded by one or more plasmid vectors. In some aspects, the plasmid vector can contain a selection marker which provides for identification and/or selection of cells which received said vector.

3. RGENs (CRISPR/Cas Systems)

In some embodiments, the disruption is carried out using one or more DNA-binding nucleic acids, such as disruption via an RNA-guided endonuclease (RGEN). For example, the disruption can be carried out using clustered regularly interspaced short palindromic repeats (CRISPR) and CRISPR-associated (Cas) proteins. In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), and/or other sequences and transcripts from a CRISPR locus.

The CRISPR/Cas nuclease or CRISPR/Cas nuclease system can include a non-coding RNA molecule (guide) RNA, which sequence-specifically binds to DNA, and a Cas protein (e.g., Cas9), with nuclease functionality (e.g., two nuclease domains). One or more elements of a CRISPR system can derive from a type I, type II, or type III CRISPR system, e.g., derived from a particular organism comprising an endogenous CRISPR system, such as Streptococcus pyogenes.

In some aspects, a Cas nuclease and gRNA (including a fusion of crRNA specific for the target sequence and fixed tracrRNA) are introduced into the cell. In general, target sites at the 5′ end of the gRNA target the Cas nuclease to the target site, e.g., the gene, using complementary base pairing. The target site may be selected based on its location immediately 5′ of a protospacer adjacent motif (PAM) sequence, such as typically NGG, or NAG. In this respect, the gRNA is targeted to the desired sequence by modifying the first 20, 19, 18, 17, 16, 15, 14, 14, 12, 11, or 10 nucleotides of the guide RNA to correspond to the target DNA sequence. In general, a CRISPR system is characterized by elements that promote the formation of a CRISPR complex at the site of a target sequence. Typically, “target sequence” generally refers to a sequence to which a guide sequence is designed to have complementarity, where hybridization between the target sequence and a guide sequence promotes the formation of a CRISPR complex. Full complementarity is not necessarily required, provided there is sufficient complementarity to cause hybridization and promote formation of a CRISPR complex.

The CRISPR system can induce double stranded breaks (DSBs) at the target site, followed by disruptions as discussed herein. In other embodiments, Cas9 variants, deemed “nickases,” are used to nick a single strand at the target site. Paired nickases can be used, e.g., to improve specificity, each directed by a pair of different gRNAs targeting sequences such that upon introduction of the nicks simultaneously, a 5′ overhang is introduced. In other embodiments, catalytically inactive Cas9 is fused to a heterologous effector domain such as a transcriptional repressor or activator, to affect gene expression.

The target sequence may comprise any polynucleotide, such as DNA or RNA polynucleotides. The target sequence may be located in the nucleus or cytoplasm of the cell, such as within an organelle of the cell. Generally, a sequence or template that may be used for recombination into the targeted locus comprising the target sequences is referred to as an “editing template” or “editing polynucleotide” or “editing sequence”. In some aspects, an exogenous template polynucleotide may be referred to as an editing template. In some aspects, the recombination is homologous recombination.

Typically, in the context of an endogenous CRISPR system, formation of the CRISPR complex (comprising the guide sequence hybridized to the target sequence and complexed with one or more Cas proteins) results in cleavage of one or both strands in or near (e.g. within 1, 2, 3, 4, 5, 6, 7, 8, 9, 10, 20, 50, or more base pairs from) the target sequence. The tracr sequence, which may comprise or consist of all or a portion of a wild-type tracr sequence (e.g. about or more than about 20, 26, 32, 45, 48, 54, 63, 67, 85, or more nucleotides of a wild-type tracr sequence), may also form part of the CRISPR complex, such as by hybridization along at least a portion of the tracr sequence to all or a portion of a tracr mate sequence that is operably linked to the guide sequence. The tracr sequence has sufficient complementarity to a tracr mate sequence to hybridize and participate in formation of the CRISPR complex, such as at least 50%, 60%, 70%, 80%, 90%, 95% or 99% of sequence complementarity along the length of the tracr mate sequence when optimally aligned.

One or more vectors driving expression of one or more elements of the CRISPR system can be introduced into the cell such that expression of the elements of the CRISPR system direct formation of the CRISPR complex at one or more target sites. Components can also be delivered to cells as proteins and/or RNA. For example, a Cas enzyme, a guide sequence linked to a tracr-mate sequence, and a tracr sequence could each be operably linked to separate regulatory elements on separate vectors. Alternatively, two or more of the elements expressed from the same or different regulatory elements, may be combined in a single vector, with one or more additional vectors providing any components of the CRISPR system not included in the first vector. The vector may comprise one or more insertion sites, such as a restriction endonuclease recognition sequence (also referred to as a “cloning site”). In some embodiments, one or more insertion sites are located upstream and/or downstream of one or more sequence elements of one or more vectors. When multiple different guide sequences are used, a single expression construct may be used to target CRISPR activity to multiple different, corresponding target sequences within a cell.

A vector may comprise a regulatory element operably linked to an enzyme-coding sequence encoding the CRISPR enzyme, such as a Cas protein. Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof. These enzymes are known; for example, the amino acid sequence of S. pyogenes Cas9 protein may be found in the SwissProt database under accession number Q99ZW2.

The CRISPR enzyme can be Cas9 (e.g., from S. pyogenes or S. pneumonia). The CRISPR enzyme can direct cleavage of one or both strands at the location of a target sequence, such as within the target sequence and/or within the complement of the target sequence. The vector can encode a CRISPR enzyme that is mutated with respect to a corresponding wild-type enzyme such that the mutated CRISPR enzyme lacks the ability to cleave one or both strands of a target polynucleotide containing a target sequence. For example, an aspartate-to-alanine substitution (D10A) in the RuvC I catalytic domain of Cas9 from S. pyogenes converts Cas9 from a nuclease that cleaves both strands to a nickase (cleaves a single strand). In some embodiments, a Cas9 nickase may be used in combination with guide sequence(s), e.g., two guide sequences, which target respectively sense and antisense strands of the DNA target. This combination allows both strands to be nicked and used to induce NHEJ or HDR.

In some embodiments, an enzyme coding sequence encoding the CRISPR enzyme is codon optimized for expression in particular cells, such as eukaryotic cells. The eukaryotic cells may be those of or derived from a particular organism, such as a mammal, including but not limited to human, mouse, rat, rabbit, dog, or non-human primate. In general, codon optimization refers to a process of modifying a nucleic acid sequence for enhanced expression in the host cells of interest by replacing at least one codon of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization.

In general, a guide sequence is any polynucleotide sequence having sufficient complementarity with a target polynucleotide sequence to hybridize with the target sequence and direct sequence-specific binding of the CRISPR complex to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more.

Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), Clustal W, Clustal X, BLAT, Novoalign (Novocraft Technologies, ELAND (Illumina, San Diego, Calif.), SOAP (available at soap.genomics.org.cn), and Maq (available at maq.sourceforge.net).

The CRISPR enzyme may be part of a fusion protein comprising one or more heterologous protein domains. A CRISPR enzyme fusion protein may comprise any additional protein sequence, and optionally a linker sequence between any two domains. Examples of protein domains that may be fused to a CRISPR enzyme include, without limitation, epitope tags, reporter gene sequences, and protein domains having one or more of the following activities: methylase activity, demethylase activity, transcription activation activity, transcription repression activity, transcription release factor activity, histone modification activity, RNA cleavage activity and nucleic acid binding activity. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporter genes include, but are not limited to, glutathione-5-transferase (GST), horseradish peroxidase (HRP), chloramphenicol acetyltransferase (CAT) beta galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP). A CRISPR enzyme may be fused to a gene sequence encoding a protein or a fragment of a protein that bind DNA molecules or bind other cellular molecules, including but not limited to maltose binding protein (MBP), S-tag, Lex A DNA binding domain (DBD) fusions, GAL4A DNA binding domain fusions, and herpes simplex virus (HSV) BP16 protein fusions.

F. Differentiation of iPSCs

In some embodiments, methods are provided for producing differentiated cells from an essentially single cell suspension of pluripotent stem cells (PSCs) such as human iPSCs. In some embodiments, the PSCs are cultured to pre-confluency to prevent any cell aggregates. In certain aspects, the PSCs are dissociated by incubation with a cell dissociation enzyme, such as exemplified by TRYPSIN™ or TRYPLE™. PSCs can also be dissociated into an essentially single cell suspension by pipetting. In addition, Blebbistatin (e.g., about 2.5 μM) can be added to the medium to increase PSC survival after dissociation into single cells while the cells are not adhered to a culture vessel. A ROCK inhibitor instead of Blebbistatin may alternatively used to increase PSC survival after dissociated into single cells.

Once a single cell suspension of PSCs is obtained at a known cell density, the cells are generally seeded in an appropriate culture vessel, such as a tissue culture plate, such as a flask, 6-well, 24-well, or 96-well plate. A culture vessel used for culturing the cell(s) can include, but is particularly not limited to: flask, flask for tissue culture, dish, petri dish, dish for tissue culture, multi dish, micro plate, micro-well plate, multi plate, multi-well plate, micro slide, chamber slide, tube, tray, CELLSTACK® Chambers, culture bag, and roller bottle, as long as it is capable of culturing the stem cells therein. The cells may be cultured in a volume of at least or about 0.2, 0.5, 1, 2, 5, 10, 20, 30, 40, 50 ml, 100 ml, 150 ml, 200 ml, 250 ml, 300 ml, 350 ml, 400 ml, 450 ml, 500 ml, 550 ml, 600 ml, 800 ml, 1000 ml, 1500 ml, or any range derivable therein, depending on the needs of the culture. In a certain embodiment, the culture vessel may be a bioreactor, which may refer to any device or system ex vivo that supports a biologically active environment such that cells can be propagated. The bioreactor may have a volume of at least or about 2, 4, 5, 6, 8, 10, 15, 20, 25, 50, 75, 100, 150, 200, 500 liters, 1, 2, 4, 6, 8, 10, 15 cubic meters, or any range derivable therein.

In certain aspects, the PSCs, such as iPSCs, are plated at a cell density appropriate for efficient differentiation. Generally, the cells are plated at a cell density of about 1,000 to about 75,000 cells/cm², such as of about 5,000 to about 40,000 cells/cm². In a 6 well plate, the cells may be seeded at a cell density of about 50,000 to about 400,000 cells per well. In exemplary methods, the cells are seeded at a cell density of about 100,000, about 150,00, about 200,000, about 250,000, about 300,000 or about 350,000 cells per well, such as about 200,00 cells per well.

The PSCs, such as iPSCs, are generally cultured on culture plates coated by one or more cellular adhesion proteins to promote cellular adhesion while maintaining cell viability. For example, preferred cellular adhesion proteins include extracellular matrix proteins such as vitronectin, laminin, collagen and/or fibronectin which may be used to coat a culturing surface as a means of providing a solid support for pluripotent cell growth. The term “extracellular matrix” is recognized in the art. Its components include one or more of the following proteins: fibronectin, laminin, vitronectin, tenascin, entactin, thrombospondin, elastin, gelatin, collagen, fibrillin, merosin, anchorin, chondronectin, link protein, bone sialoprotein, osteocalcin, osteopontin, epinectin, hyaluronectin, undulin, epiligrin, and kalinin. In exemplary methods, the PSCs are grown on culture plates coated with vitronectin or fibronectin. In some embodiments, the cellular adhesion proteins are human proteins.

The extracellular matrix (ECM) proteins may be of natural origin and purified from human or animal tissues or, alternatively, the ECM proteins may be genetically engineered recombinant proteins or synthetic in nature. The ECM proteins may be a whole protein or in the form of peptide fragments, native or engineered. Examples of ECM protein that may be useful in the matrix for cell culture include laminin, collagen I, collagen IV, fibronectin and vitronectin. In some embodiments, the matrix composition includes synthetically generated peptide fragments of fibronectin or recombinant fibronectin. In some embodiments, the matrix composition is xeno-free. For example, in the xeno-free matrix to culture human cells, matrix components of human origin may be used, wherein any non-human animal components may be excluded.

In some aspects, the total protein concentration in the matrix composition may be about 1 ng/mL to about 1 mg/mL. In some preferred embodiments, the total protein concentration in the matrix composition is about 1 μg/mL to about 300 m/mL. In more preferred embodiments, the total protein concentration in the matrix composition is about 5 μg/mL to about 200 m/mL.

Cells can be cultured with the nutrients necessary to support the growth of each specific population of cells. Generally, the cells are cultured in growth media including a carbon source, a nitrogen source and a buffer to maintain pH. The medium can also contain fatty acids or lipids, amino acids (such as non-essential amino acids), vitamin(s), growth factors, cytokines, antioxidant substances, pyruvic acid, buffering agents, and inorganic salts. An exemplary growth medium contains a minimal essential media, such as Dulbecco's Modified Eagle's medium (DMEM) or ESSENTIAL 8™ (E8™) medium, supplemented with various nutrients, such as non-essential amino acids and vitamins, to enhance stem cell growth. Examples of minimal essential media include, but are not limited to, Minimal Essential Medium Eagle (MEM) Alpha medium, Dulbecco's modified Eagle medium (DMEM), RPMI-1640 medium, 199 medium, and F12 medium. Additionally, the minimal essential media may be supplemented with additives such as horse, calf or fetal bovine serum. Alternatively, the medium can be serum free. In other cases, the growth media may contain “knockout serum replacement,” referred to herein as a serum-free formulation optimized to grow and maintain undifferentiated cells, such as stem cell, in culture. KNOCKOUT™ serum replacement is disclosed, for example, in U.S. Patent Application No. 2002/0076747, which is incorporated herein by reference. Preferably, the PSCs are cultured in a fully defined and feeder free media.

Accordingly, the PSCs are generally cultured in a fully defined culture medium after plating. In certain aspects, about 18-24 hours after seeding, the medium is aspirated and fresh medium, such as E8™ medium, is added to the culture. In certain aspects, the single cell PSCs are cultured in the fully defined culture medium for about 1, 2 or 3 days after plating. Preferably, the single cells PSCs are cultured in the fully defined culture medium for about 2 days before proceeding with the differentiation process.

In some embodiments, the medium may contain or may not contain any alternatives to serum. The alternatives to serum can include materials which appropriately contain albumin (such as lipid-rich albumin, albumin substitutes such as recombinant albumin, plant starch, dextrans and protein hydrolysates), transferrin (or other iron transporters), fatty acids, insulin, collagen precursors, trace elements, 2-mercaptoethanol, 3′-thiolgiycerol, or equivalents thereto. The alternatives to serum can be prepared by the method disclosed in International Publication No. WO 98/30679, for example. Alternatively, any commercially available materials can be used for more convenience. The commercially available materials include KNOCKOUT™ Serum Replacement (KSR), Chemically-defined Lipid concentrated (Gibco), and GLUTAMAX™ (Gibco).

Other culturing conditions can be appropriately defined. For example, the culturing temperature can be about 30 to 40° C., for example, at least or about 31, 32, 33, 34, 35, 36, 37, 38, 39° C. but particularly not limited to them. In one embodiment, the cells are cultured at 37° C. The CO₂ concentration can be about 1 to 10%, for example, about 2 to 5%, or any range derivable therein. The oxygen tension can be at least, up to, or about 1, 2, 3, 4, 5, 6, 7, 8, 9, 10, 20%, or any range derivable therein.

G. Cryopreservation of iPSCs or Differentiated Cells

The cells produced by the methods disclosed herein can be cryopreserved, see for example, PCT Publication No. 2012/149484 A2, which is incorporated by reference herein. The cells can be cryopreserved with or without a substrate. In several embodiments, the storage temperature ranges from about −50° C. to about −60° C., about −60° C. to about −70° C., about −70° C. to about −80° C., about −80° C. to about −90° C., about −90° C. to about −100° C., and overlapping ranges thereof. In some embodiments, lower temperatures are used for the storage (e.g., maintenance) of the cryopreserved cells. In several embodiments, liquid nitrogen (or other similar liquid coolant) is used to store the cells. In further embodiments, the cells are stored for greater than about 6 hours. In additional embodiments, the cells are stored about 72 hours. In several embodiments, the cells are stored 48 hours to about one week. In yet other embodiments, the cells are stored for about 1, 2, 3, 4, 5, 6, 7, or 8 weeks. In further embodiments, the cells are stored for 1, 2, 3, 4, 5, 67, 8, 9, 10, 11 or 12 months. The cells can also be stored for longer times. The cells can be cryopreserved separately or on a substrate, such as any of the substrates disclosed herein.

In some embodiments, additional cryoprotectants can be used. For example, the cells can be cryopreserved in a cryopreservation solution comprising one or more cryoprotectants, such as DM80, serum albumin, such as human or bovine serum albumin. In certain embodiments, the solution comprises about 1%, about 1.5%, about 2%, about 2.5%, about 3%, about 4%, about 5%, about 6%, about 7%, about 8%, about 9%, or about 10% DMSO. In other embodiments, the solution comprises about 1% to about 3%, about 2% to about 4%, about 3% to about 5%, about 4% to about 6%, about 5% to about 7%, about 6% to about 8%, about 7% to about 9%, or about 8% to about 10% dimethylsulfoxide (DMSO) or albumin. In a specific embodiment, the solution comprises 2.5% DMSO. In another specific embodiment, the solution comprises 10% DMSO.

Cells may be cooled, for example, at about 1° C. minute during cryopreservation. In some embodiments, the cryopreservation temperature is about −80° C. to about −180° C., or about −125° C. to about −140° C. In some embodiments, the cells are cooled to 4° C. prior to cooling at about 1° C./minute. Cryopreserved cells can be transferred to vapor phase of liquid nitrogen prior to thawing for use. In some embodiments, for example, once the cells have reached about −80° C., they are transferred to a liquid nitrogen storage area. Cryopreservation can also be done using a controlled-rate freezer. Cryopreserved cells may be thawed, e.g., at a temperature of about 25° C. to about 40° C., and typically at a temperature of about 37° C.

### III. USE OF ENGINEERED CELL LINES

Certain aspects provide a method to produce a cell line with stable transgene expression which can be used for a number of important research, development, and commercial purposes.

The cell lines produced by the methods disclosed herein may be used in any methods and applications currently known in the art iPSCs or differentiated cells. For example, a method of assessing a compound may be provided, comprising assaying a pharmacological or toxicological property of the compound on the cell line. There may also be provided a method of assessing a compound for an effect on a cell culture, comprising: a) contacting the cell culture provided herein with the compound; and b) assaying an effect of the compound on the cell culture.

A. Test Compound Screening

The cell culture can be used commercially to screen for factors (such as solvents, small molecule drugs, peptides, oligonucleotides) or environmental conditions (such as culture conditions or manipulation) that affect the characteristics of such cells and their various progeny. For example, test compounds may be chemical compounds, small molecules, polypeptides, growth factors, cytokines, or other biological agents.

In one embodiment, a method includes contacting a cell culture with a test agent and determining if the test agent modulates activity or function of cells within the population. In some applications, screening assays are used for the identification of agents that modulate cell proliferation, alter cell differentiation, or affect cell viability. Screening assays may be performed in vitro or in vivo. Methods of screening and identifying candidate agents include those suitable for high-throughput screening. For example, the cell culture can be positioned or placed on a culture dish, flask, roller bottle or plate (e.g., a single multi-well dish or dish such as 8, 16, 32, 64, 96, 384 and 1536 multi-well plate or dish), optionally at defined locations, for identification of potentially therapeutic molecules. Libraries that can be screened include, for example, small molecule libraries, siRNA libraries, and adenoviral transfection vector libraries.

Other screening applications relate to the testing of pharmaceutical compounds for their effect on retinal tissue maintenance or repair. Screening may be done either because the compound is designed to have a pharmacological effect on the cells, or because a compound designed to have effects elsewhere may have unintended side effects on cells of this tissue type.

B. Therapy and Transplantation

Other embodiments can also provide use of the cell lines for the treatment of a disease or disorder. In another aspect, the disclosure provides a method of treatment of an individual in need thereof, comprising administering a composition comprising engineered cells to said individual.

To determine suitability of cell compositions for therapeutics administration, the cells can first be tested in a suitable animal model. In one aspect, the cell lines are evaluated for their ability to survive and maintain their phenotype in vivo. The compositions are transplanted to immunodeficient animals (e.g., nude mice or animals rendered immunodeficient chemically or by irradiation). Tissues are harvested after a period of growth, and assessed as to whether the pluripotent stem cell-derived cells are still present.

As used herein, a disease or disorder refers to a pathological condition in an organism resulting from, for example, infection or genetic defect, and characterized by identifiable symptoms. An exemplary disease as described herein is a neoplastic disease, such as cancer. As used herein, neoplastic disease refers to any disorder involving cancer, including tumor development, growth, metastasis and progression.

As used herein, cancer is a term for diseases caused by or characterized by any type of malignant tumor, including metastatic cancers, lymphatic tumors, and blood cancers. Exemplary cancers include, but are not limited to, leukemia, lymphoma, pancreatic cancer, lung cancer, ovarian cancer, breast cancer, cervical cancer, bladder cancer, prostate cancer, glioma tumors, adenocarcinomas, liver cancer and skin cancer. Exemplary cancers in humans include a bladder tumor, breast tumor, prostate tumor, basal cell carcinoma, biliary tract cancer, bladder cancer, bone cancer, brain and CNS cancer (e.g., glioma tumor), cervical cancer, choriocarcinoma, colon and rectum cancer, connective tissue cancer, cancer of the digestive system; endometrial cancer, esophageal cancer; eye cancer; cancer of the head and neck; gastric cancer; intra-epithelial neoplasm; kidney cancer; larynx cancer; leukemia; liver cancer; lung cancer (e.g., small cell and non-small cell); lymphoma including Hodgkin's and Non-Hodgkin's lymphoma; melanoma; myeloma, neuroblastoma, oral cavity cancer (e.g., lip, tongue, mouth, and pharynx); ovarian cancer; pancreatic cancer, retinoblastoma; rhabdomyosarcoma; rectal cancer, renal cancer, cancer of the respiratory system; sarcoma, skin cancer; stomach cancer, testicular cancer, thyroid cancer; uterine cancer, cancer of the urinary system, as well as other carcinomas and sarcomas. Exemplary cancers commonly diagnosed in dogs, cats, and other pets include, but are not limited to, lymphosarcoma, osteosarcoma, mammary tumors, mastocytoma, brain tumor, melanoma, adenosquamous carcinoma, carcinoid lung tumor, bronchial gland tumor, bronchiolar adenocarcinoma, fibroma, myxochondroma, pulmonary sarcoma, neurosarcoma, osteoma, papilloma, retinoblastoma, Ewing's sarcoma, Wilm's tumor, Burkitt's lymphoma, microglioma, neuroblastoma, osteoclastoma, oral neoplasia, fibrosarcoma, osteosarcoma and rhabdomyosarcoma, genital squamous cell carcinoma, transmissible venereal tumor, testicular tumor, seminoma, Sertoli cell tumor, hemangiopericytoma, histiocytoma, chloroma (e.g., granulocytic sarcoma), corneal papilloma, corneal squamous cell carcinoma, hemangiosarcoma, pleural mesothelioma, basal cell tumor, thymoma, stomach tumor, adrenal gland carcinoma, oral papillomatosis, hemangioendothelioma and cystadenoma, follicular lymphoma, intestinal lymphosarcoma, fibrosarcoma and pulmonary squamous cell carcinoma. Exemplary cancers diagnosed in rodents, such as a ferret, include, but are not limited to, insulinoma, lymphoma, sarcoma, neuroma, pancreatic islet cell tumor, gastric MALT lymphoma and gastric adenocarcinoma. Exemplary neoplasias affecting agricultural livestock include, but are not limited to, leukemia, hemangiopericytoma and bovine ocular neoplasia (in cattle); preputial fibrosarcoma, ulcerative squamous cell carcinoma, preputial carcinoma, connective tissue neoplasia and mastocytoma (in horses); hepatocellular carcinoma (in swine); lymphoma and pulmonary adenomatosis (in sheep); pulmonary sarcoma, lymphoma, Rous sarcoma, reticulo-endotheliosis, fibrosarcoma, nephroblastoma, B-cell lymphoma and lymphoid leukosis (in avian species); retinoblastoma, hepatic neoplasia, lymphosarcoma (lymphoblastic lymphoma), plasmacytoid leukemia and swimbladder sarcoma (in fish), caseous lymphadenitis (CLA): chronic, infectious, contagious disease of sheep and goats caused by the bacterium Corynebacterium pseudotuberculosis, and contagious lung tumor of sheep caused by jaagsiekte.

Pharmaceutical compositions of the cell lines produced by the methods disclosed herein are also provided. These compositions can include at least about 1×10³ cells, about 1×10⁴ cells, about 1×10⁵ cells, about 1×10⁶ cells, about 1×10⁷ cells, about 1×10⁸ cells, or about 1×10⁹ cells. In certain embodiments, the compositions are substantially purified preparations comprising differentiated cells produced by the methods disclosed herein. Compositions are also provided that include a scaffold, such as a polymeric carrier and/or an extracellular matrix, and an effective amount of the cells produced by the methods disclosed herein. The matrix material is generally physiologically acceptable and suitable for use in in vivo applications. For example, the physiologically acceptable materials include, but are not limited to, solid matrix materials that are absorbable and/or non-absorbable, such as small intestine submucosa (SIS), crosslinked or non-crosslinked alginate, hydrocolloid, foams, collagen gel, collagen sponge, polyglycolic acid (PGA) mesh, fleeces and bioadhesives.

Suitable polymeric carriers also include porous meshes or sponges formed of synthethic or natural polymers, as well as polymer solutions. For example, the matrix is a polymeric mesh or sponge, or a polymeric hydrogel. Natural polymers that can be used include proteins such as collagen, albumin, and fibrin; and polysaccharides such as alginate and polymers of hyaluronic acid. Synthetic polymers include both biodegradable and non-biodegradable polymers. For example, biodegradable polymers include polymers of hydroxy acids such as polyactic acid (PLA), polyglycolic acid (PGA) and polylactic acid-glycolic acid (PGLA), polyorthoesters, polyanhydrides, polyphosphazenes, and combinations thereof. Non-biodegradable polymers include polyacrylates, polymethacrylates, ethylene vinyl acetate, and polyvinyl alcohols.

Polymers that can form ionic or covalently crosslinked hydrogels which are malleable can be used. A hydrogel is a substance formed when an organic polymer (natural or synthetic) is cross-linked via covalent, ionic, or hydrogen bonds to create a three-dimensional open-lattice structure which entraps water molecules to form a gel. Examples of materials which can be used to form a hydrogel include polysaccharides such as alginate, polyphosphazines, and polyacrylates, which are crosslinked ionically, or block copolymers such as PLURON1CS™ or TETRON1CS™, polyethylene oxide-polypropylene glycol block copolymers which are crosslinked by temperature or H, respectively. Other materials include proteins such as fibrin, polymers such as polyvinylpyrrolidone, hyaluronic acid and collagen.

C. Distribution for Commercial, Therapeutic, and Research Purposes

In some embodiments, a reagent system is provided that includes a set or combination of cells that exists at any time during manufacture, distribution or use. The culture sets comprise any combination of the cell population described herein in combination with undifferentiated pluripotent stem cells or other differentiated cell types, often sharing the same genome. Each cell type may be packaged together, or in separate containers in the same facility, or at different locations, at the same or different times, under control of the same entity or different entities sharing a business relationship.

Pharmaceutical compositions may optionally be packaged in a suitable container with written instructions for a desired purpose, such as the reconstitution of cell function to improve a disease or injury of tissue.

### IV. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Codon Optimization to Prevent Gene Silencing

To test if methylation was the cause of transgene silencing, all CpG motifs were removed from coding regions of genes which were known (e.g., GFP) to be silenced or speculated to be silenced (e.g., PuroR and NeoR). Due to the ease of visual inspection, the concept was rigorously tested comparing WT AcGFP1 and CpG-free AcGFP1. There was no change in the amino acid sequence after removal of the CpG motifs between SEQ ID NO:13 and SEQ ID NO:14.

AcGFP1 DNA sequence (SEQ ID NO: 13):
atggtgagcaagggCGcCGagctgttcacCGgcatCGtgcccatcctgat
CGagctgaatggCGatgtgaatggccacaagttcagCGtgagCGgCGagg
gCGagggCGatgccacctaCGgcaagctgaccctgaagttcatctgcacc
acCGgcaagctgcctgtgccctggcccaccctggtgaccaccctgagcta
CGgCGtgcagtgcttctcaCGctacccCGatcacatgaagcagcaCGact
tcttcaagagCGccatgcctgagggctacatccaggagCGcaccatcttc
ttCGaggatgaCGgcaactacaagtCGCGCGcCGaggtgaagttCGaggg
CGataccctggtgaatCGcatCGagctgacCGgcacCGatttcaaggagg
atggcaacatcctgggcaataagatggagtacaactacaaCGcccacaat
gtgtacatcatgacCGacaaggccaagaatggcatcaaggtgaacttcaa
gatcCGccacaacatCGaggatggcagCGtgcagctggcCGaccactacc
agcagaatacccccatCGgCGatggccctgtgctgctgccCGataaccac
tacctgtccacccagagCGccctgtccaaggaccccaaCGagaagCGCGa
tcacatgatctacttCGgcttCGtgacCGcCGcCGccatcacccaCGgca
tggatgagctgtacaagTAA
CpG-free AcGFP1 DNA sequence (SEQ ID NO: 14):
ATGGTGAGCAAGGGCGCCGAGCTGTTCACCGGCATCGTGCCCATCCTGAT
CGAGCTGAATGGCGATGTGAATGGCCACAAGTTCAGCGTGAGCGGCGAGG
GCGAGGGCGATGCCACCTACGGCAAGCTGACCCTGAAGTTCATCTGCACC
ACCGGCAAGCTGCCTGTGCCCTGGCCCACCCTGGTGACCACCCTGAGCTA
CGGCGTGCAGTGCTTCTCACGCTACCCCGATCACATGAAGCAGCACGACT
TCTTCAAGAGCGCCATGCCTGAGGGCTACATCCAGGAGCGCACCATCTTC
TTCGAGGATGACGGCAACTACAAGTCGCGCGCCGAGGTGAAGTTCGAGGG
CGATACCCTGGTGAATCGCATCGAGCTGACCGGCACCGATTTCAAGGAGG
ATGGCAACATCCTGGGCAATAAGATGGAGTACAACTACAACGCCCACAAT
GTGTACATCATGACCGACAAGGCCAAGAATGGCATCAAGGTGAACTTCAA
GATCCGCCACAACATCGAGGATGGCAGCGTGCAGCTGGCCGACCACTACC
AGCAGAATACCCCCATCGGCGATGGCCCTGTGCTGCTGCCCGATAACCAC
TACCTGTCCACCCAGAGCGCCCTGTCCAAGGACCCCAACGAGAAGCGCGA
TCACATGATCTACTTCGGCTTCGTGACCGCCGCCGCCATCACCCACGGCA
TGGATGAGCTGTACAAG

The DNA sequence of the genes of interest were meticulously modified to remove all CG motifs replacing with codons which were 1) not rare, 2) did not generate stretches of mononucleotide stretches and 3) maintained a % GC content similar to the WT version of the gene. For example, for CpG-free AcGFP1, the WT version of AcGFP1 (SEQ ID NO:13) has a 59% GC content and the new CpG-free AcGFP1 (SEQ ID NO:14) has a 52% GC content.

The EEF1A1 promoter was used for expression of AcGFP1 in iPSCs at the PPP1R12C locus. Testing of the CpG-free AcGFP1, compared to the WT AcGFP1, revealed that silencing of gene expression was overcome by removing the CpGs in the protein coding sequence (FIG. 3).

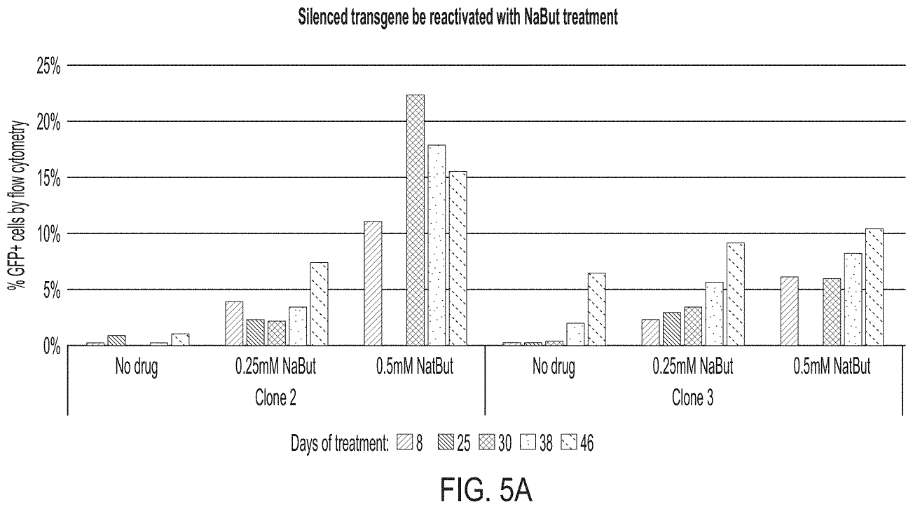

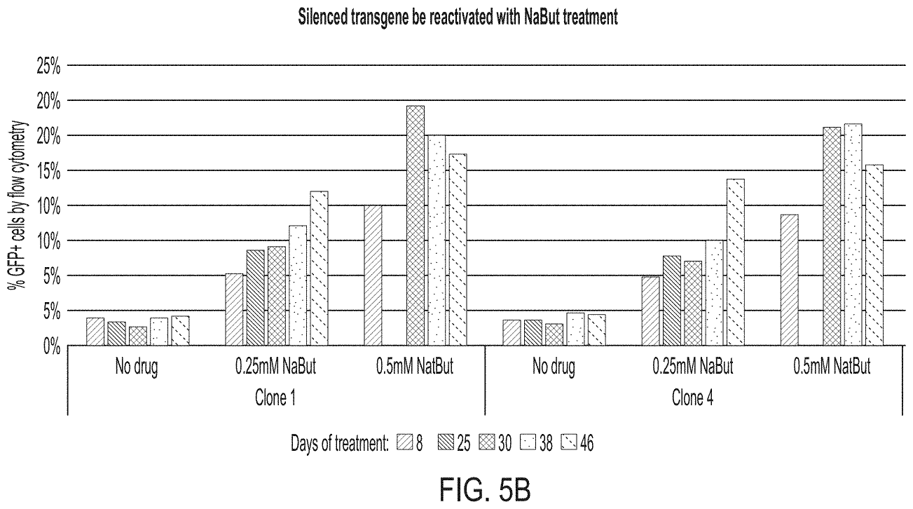

However, after 5 months, a small percentage of cells were detected with no GFP expression (3%) even though the CpGs had been removed from AcGFP1. To investigate this population, clones were isolated by single cell sorting for no GFP expression. The cells were treated with sodium butyrate (NaBut), a histone deacetylase (HDAC) inhibitor capable of removing chromatin structure and inducing demethylation. It was observed that NaBut treatment resulted in a dose dependent reactivation of GFP expression (FIG. 5).

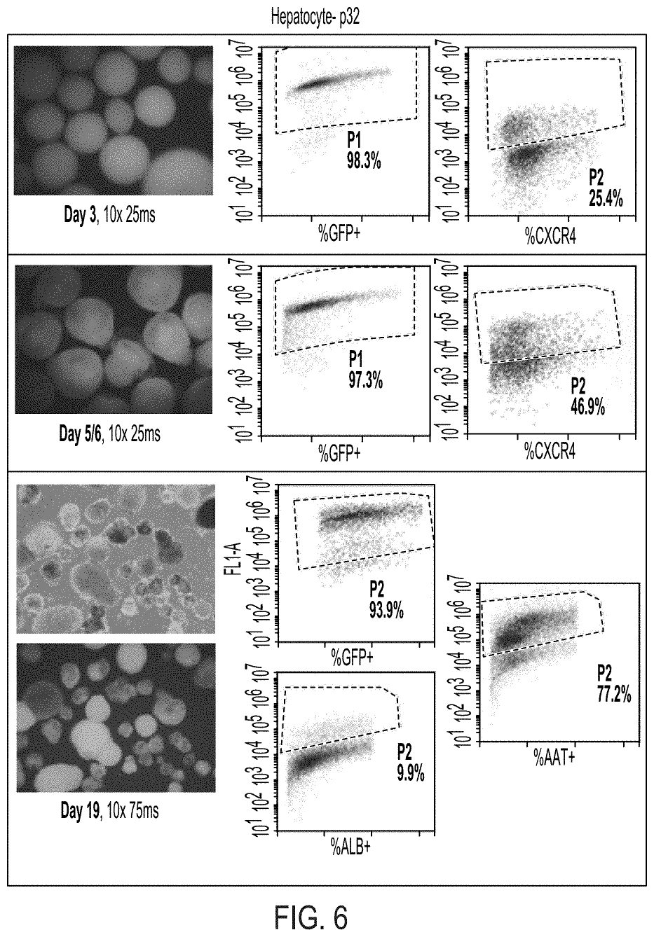

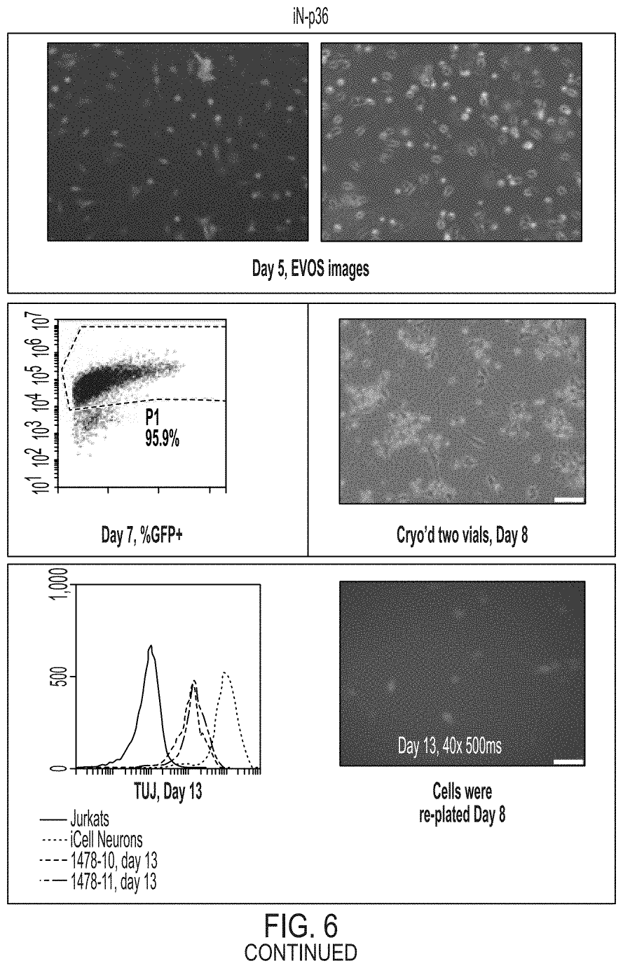

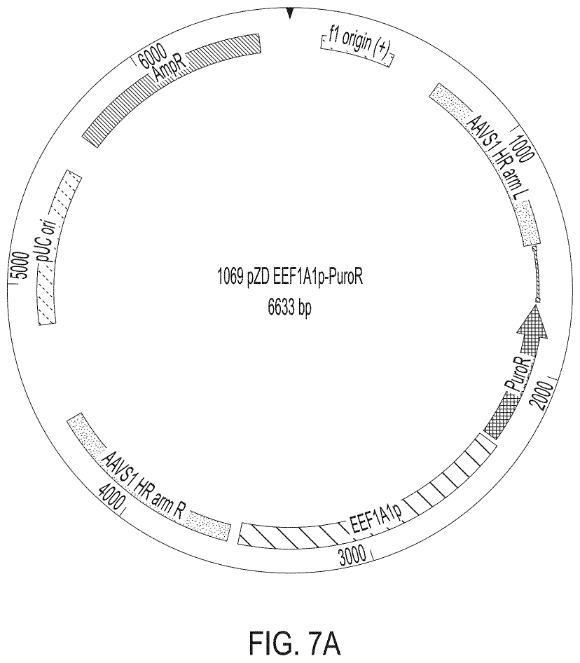

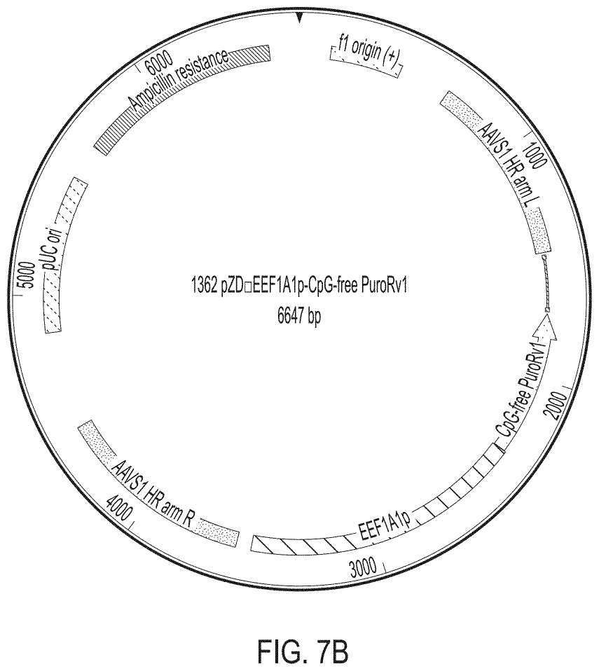

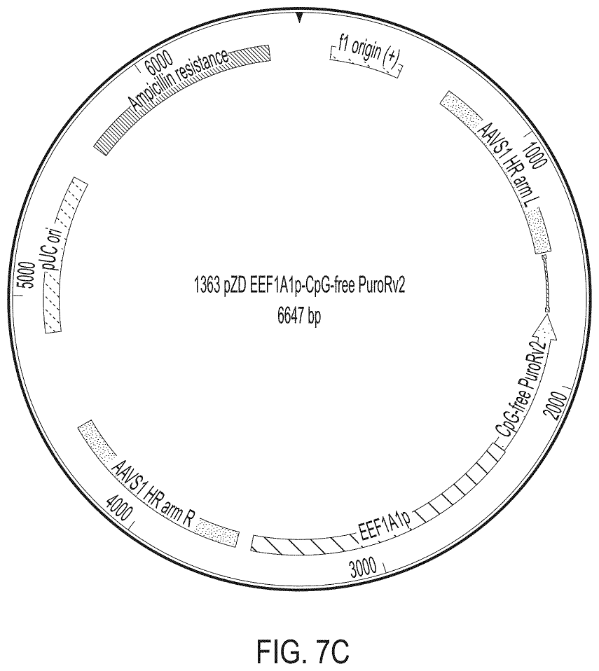

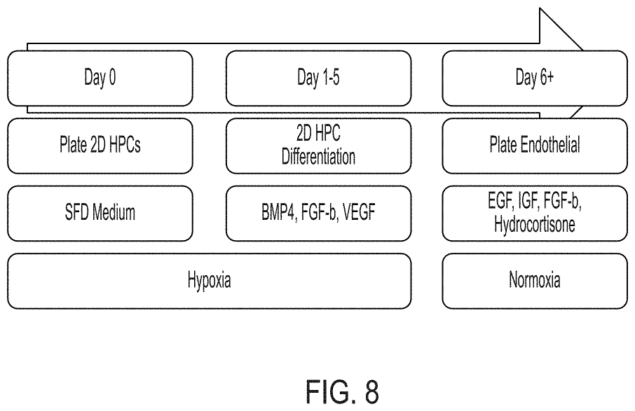

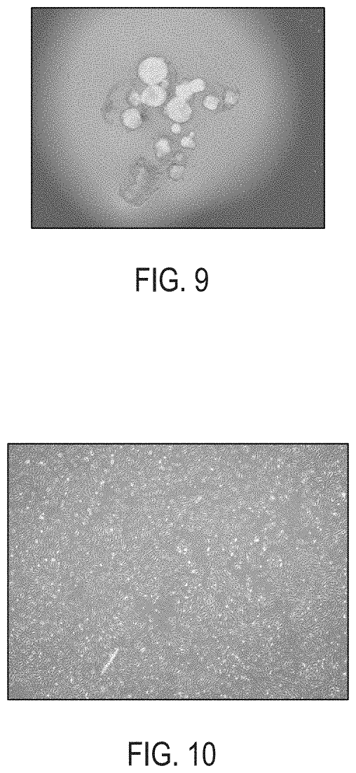

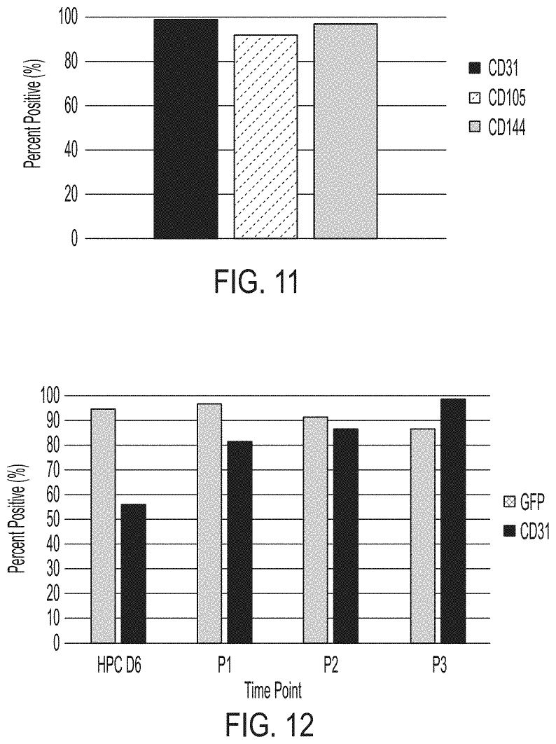

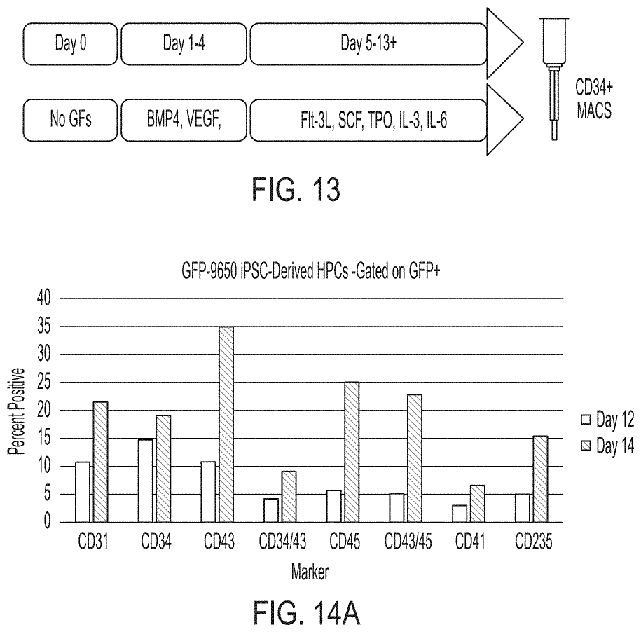

The CpG-free AcGFP1 iPSCs were differentiated to hepatocytes or neurons and a high percentage of GFP-positive differentiated cells were observed (FIG. 6).

To validate the results, codon optimization of PuroRv1 (synthesized based on Invivogen amino acid sequence) in pUC57-KanR(m) was performed. The CpG-free sequence of PuroR (SEQ ID NO:15) is shown below.

Plasmid 1346 CpG-free PuroR:
(SEQ ID NO: 15)
ATGACTGAATACAAACCAACTGTTAGACTGGCAACTAGAGATGATGTTCC
AAGAGCAGTTAGAACCCTGGCTGCTGCATTTGCTGACTACCCTGCAACCA
GACACACTGTGGACCCAGACAGACACATTGAAAGAGTGACTGAACTGCAG
GAGCTGTTCCTGACCAGAGTGGGCCTGGACATTGGCAAAGTGTGGGTGGC
AGATGATGGTGCTGCTGTGGCAGTGTGGACCACCCCTGAATCTGTTGAAG
CTGGTGCAGTGTTTGCTGAGATTGGCCCAAGAATGGCAGAACTGTCTGGC
AGCAGACTGGCAGCACAACAGCAGATGGAAGGTCTGCTGGCACCACACAG
ACCAAAAGAACCTGCTTGGTTCCTGGCAACTGTGGGTGTGAGCCCTGACC
ACCAGGGTAAGGGCCTGGGCTCTGCAGTGGTGCTGCCTGGTGTGGAAGCA
GCTGAAAGAGCAGGTGTGCCTGCTTTCCTGGAGACCTCAGCTCCAAGAAA
CCTGCCTTTCTATGAAAGACTGGGCTTCACTGTGACTGCTGATGTGGAAG
TGCCAGAAGGCCCAAGAACTTGGTGCATGACTAGAAAACCAGGTGCTTGA
TAATGA
CpG-free PuroRv2 in plasmids 1347 and 1363:
(SEQ ID NO: 16)
ATGACTGAATACAAACCAACTGTTAGACTGGCAACTAGAGATGATGTTCC
AAGAGCAGTTAGAACCCTGGCTGCTGCATTTGCTGACTACCCTGCAACCA
GACACACTGTGGACCCAGACAGACACATTGAAAGAGTGACTGAACTGCAG
GAGCTGTTCCTGACCAGAGTGGGCCTGGACATTGGCAAAGTGTGGGTGGC
AGATGATGGTGCTGCTGTGGCAGTGTGGACCACCCCTGAATCTGTTGAAG
CTGGTGCAGTGTTTGCTGAGATTGGCCCAAGAATGGCAGAACTGTCTGGC
AGCAGACTGGCAGCACAACAGCAGATGGAAGGTCTGCTGGCACCACACAG
ACCAAAAGAACCTGCTTGGTTCCTGGCAACTGTGGGTGTGAGCCCTGACC
ACCAGGGTAAGGGCCTGGGCTCTGCAGTGGTGCTGCCTGGTGTGGAAGCA
GCTGAAAGAGCAGGTGTGCCTGCTTTCCTGGAGACCTCAGCTCCAAGAAA
CCTGCCTTTCTATGAAAGACTGGGCTTCACTGTGACTGCTGATGTGGAAT
GCCCAAAGGACAGAGCAACTTGGTGCATGACTAGAAAACCAGGTGCTTGA
TAATGA

The CpG-free PuroR cassette was introduced into iPSCs by electroporation.

It was observed that the cells with CpG-free PuroRv1 and PuroRv2 were capable of conferring drug resistance.

TABLE 2
Drug resistance of WT PuroR vs. CpG-free PuroR.

| | | mTeSR1 +0.1 μg/ml | mTeSR1 + 0.3 μg/ml |
|---|---|---|---|
| Sample | mTeSR1 | Puromycin | Puromycin |
| 2.038 | +++ | + | − |
| 2.038 transfected | +++ | +++ | ++ |
| with 1036 (WT | | | |
| PuroR) | | | |
| 2.038 transfected | +++ | +++ | +++ |
| with 1069 (WT | | | |
| PuroR) | | | |
| 2.038 transfected | +++ | +++ | +++ |
| with 1362 (CpG- | | | |
| free PuroRv1) | | | |
| 2.038 transfected | +++ | +++ | − |
| with 1363 (CpG- | | | |
| free PuroRv2) | | | |

The iPSC line 2.038 was transfected with plasmids encoding the puromycin gene (WT or CpG-free) driven by a constitutive promoter. The growth of iPSCs was scored on a 0 (−) to 3 (+++) scale when fed with mTeSR1 alone, mTeSR1 with 0.1 ug/mL puromycin or mTeSR1 with 0.3 ug/mL Puromycin. An untransfected cell line (2.038) was used as a control puromycin treatment control.

TABLE 3
WT PuroR vs. CpG-free PuroR.

| | | | | | | ZFN | | |
|---|---|---|---|---|---|---|---|---|
| | | | | | | plasmid | ZFN | ZFN |
| | | | | | | amount | left | right |
| Nucleo- | Targeting | Amount | Size | Conc. | Volume | each | volume | volume |
| fection | Vector ID | (μg/kb) | (kb) | (μg/μL) | (μL) | (μg) | (μl) | (μl) |
| 1 | 1069 | 2 | 8.1 | 1.15 | 11 | 2.9 | 0 | 0 |
| 2 | 1069 | 2 | 8.1 | 1.15 | 11 | 2.9 | 1 | 1 |
| 3 | 1362 | 2 | 6.3 | 1.2 | 11 | 2.9 | 0 | 0 |
| 4 | 1362 | 2 | 6.6 | 1.2 | 11 | 2.9 | 1 | 1 |
| 5 | 1363 | 2 | 6.6 | 1.57 | 8.4 | 2.9 | 0 | 0 |
| 6 | 1363 | 2 | 6.6 | 1.57 | 8.4 | 2.9 | 1 | 1 |

TABLE 4
Viability of WT PuroR vs. CpG-free PuroR on day 4 post-
electroporation and 3 days of selection.

| | | mTeSR1 +0.1 μg/ml | mTeSR1 + 0.3 μg/ml |
|---|---|---|---|
| Sample | mTeSR1 | Puromycin | Puromycin |
| 1069 − | +++ | +++ | + |
| 1069 + | +++ | +++ | +++ |
| 1362 − | +++ | +++ | + |
| 1362 + | +++ | +++ | +++ |
| 1363 − | +++ | +++ | − |
| 1363 + | +++ | +++ | − |

The iPSC line 2.038 was transfected with plasmids encoding the puromycin gene (WT or CpG-free) driven by a constitutive promoter. The growth of iPSCs was scored on a 0 (−) to 3 (+++) scale when fed with mTeSR1 alone, mTeSR1 with 0.1 ug/mL puromycin or mTeSR1 with 0.3 ug/mL Puromycin. An untransfected cell line (2.038) was used as a control puromycin treatment control.

TABLE 5
Clones screened for verification of correct genome engineering
without off-target integration or mutations at AAVSI cut site. Backbone
PCR was performed to confirm no off-target integration of the plasmid.

| | | | | Right Arm | BackBone |
|---|---|---|---|---|---|
| Plasmid | # of Clones | WT PCT | Left Arm PCR | PCR | PCR |
| 1036 | 24 | 3/24 positive | 3/3 positive | 3/3 positive | ⅔ |
| | | | | | negative at |
| | | | | | passage 4 |
| | 2013 0709 | 2013 0808 | 2013 0809 Left | 2013 0822 | 2013 0827 |
| | Nucleofection of | WT PCR | Arm PCR 1036 | Right Arm | Backbone |
| | 2.038 with | 1036 & 1362 | & 1362 Clones | PCR 1036 & | PCR 1036 |
| | plasmids 1362 & | Clones | | 1362 Clones | & 1362 |
| | 1363 | | | | Clones |
| 1362 | 24 | 11/24 | 8/11 positive | 8/11 positive | 4/7 |
| | | positive | | | negative at |
| | | | | | passage 4 |
| | 2013 0709 | 2013 0808 | 2013 0809 Left | 2013 0822 | 2013 0827 |
| | Nucleofection of | WT PCR | Arm PCR 1036 | RightArm | Backbone |
| | 2.038 with | 1036 & 1362 | & 1362 Clones | PCR 1036 & | PCR 1036 |
| | plasmids 1362 & | Clones | | 1362 Clones | & 1362 |
| | 1363 | | | | Clones |

TABLE 6
Plasmids used to engineer cell lines.
1800 pZD EEF1A1p-CpG-free AcGFP1
1800 pZD EEF1A1p-CpG-free AcGFP1
1393 pZD EEF1A1p-AcGFP1
1184 pZD-EEF1A1p-mRFP1/PGKp-puroR
1036 pZDonor AAVS1 EEF1A1p-ZsGreen/PGKp-puroR
1069 pZD EEF1A1p-Puro
1362 pZD EEF1A1p-CpG-free PuroRv1
1363 pZD EFxp-CpG-free PuroRv2

These results suggest that CpG plays a significant role in transgene silencing in iPSC lines. In addition, these results suggest global methylation or other epigenetic dysregulation plays an important role in iPSCs with defective differentiation. Thus, the present methods of optimization to remove some or all CpG motifs can be used to prevent transgene silencing.

### Example 2—Differentiation of CpG Optimized iPSCs

iPSC transfected with CpG-free AcGFP1 and mRFP1 retained expression of the fluorochromes constitutively for many passages in culture. The next step was to check the retention of the fluorochromes during differentiation of iPSCs to progenitor cells as well as end stage lineages from engineered iPSCs. It was shown that engineered iPSCs transfected with CpG-free plasmids successfully generated a pure population of endothelial cells, hematopoietic cells, macrophages and microglia.

Generation of iPSC derived endothelial cells from 9650 GFP iPSCs: Undifferentiated 9650-GFP were iPSCs maintained on MATRIGEL™ or Vitronectin in the presence of E8 and adapted to hypoxia for at least 5-10 passages. To initiate endothelial differentiation, sub-confluent iPSCs were harvested and plated at a density of 0.25 million cells/well onto Pure coat Amine culture dishes in the presence Serum Free Defined (SFD) media (Table 5) supplemented with 5 uM blebbistatin or 1 uM H1152 under hypoxic conditions. 24 hours post plating the cells were placed in SFD media supplemented with 50 ng/ml of BMP4, VEGF and FGF-b, known as SFDEB #1 Medium (Table 7). The cells were fed every 48 hours for 4-6 days to generate hematoendothelial progenitor cells. These progenitor cells can by cryopreserved or replated on a tissue culture treated plastic surface at a density of 10 k/cm² under normoxic conditions to initiate endothelial differentiation in the presence of SFD based Endothelial Medium (Table 7) with H1152.

In an exemplary method, cryopreserved day 6 hematoendothelial cells or live cultures were plated at 10 k/cm² on a tissue culture treated plastic surface in the presence of SFD based Endothelial Medium with 1 uM H1152 and normoxic conditions. The cells were given a fresh feed of endothelial medium 24 hours post plating and fed every 48 hours until they reached confluency. It took 5-6 days in culture for cells to reach confluency. The cells were harvested using TrypLE Select, stained for surface endothelial markers CD31, CD105 and CD144 and replated onto a tissue culture treated plastic at 10 k/cm² with endothelial medium and placed in normoxic incubator conditions to expand and propagate a pure population of endothelial cells.

TABLE 7
Exemplary media formulations to generate iPSC derived
endothelial cells.

| SFD | | SFD EB + 1 Medium | | SFD Endothelial Medium | |
|---|---|---|---|---|---|
| IMDM | 75% | SFD | 100% | SFD | 100% |
| Ham’s F12 | 25% | BMP4 | 50 ng/mL | EGF | 25 ng/mL |
| N2 (CTS) | 0.5% | FGF-b | 50 ng/mL | IGF | 25 ng/mL |
| B27 w/o RA | 1% | VEGF | 50 ng/mL | FGF-b | 50 ng/mL |
| (CTS) | | | | | |

| | | | |
|---|---|---|---|
| HSA | 0.05% | Hydro- | 1 μg/mL |
| | | cortisone | |

| | | |
|---|---|---|
| MTG | 450 | μM |
| Ascorbic | 50 | μg/mL |

| | |
|---|---|
| Acid | |
| Pen/Strep | 1% |
| GlutaMAX | 1% |

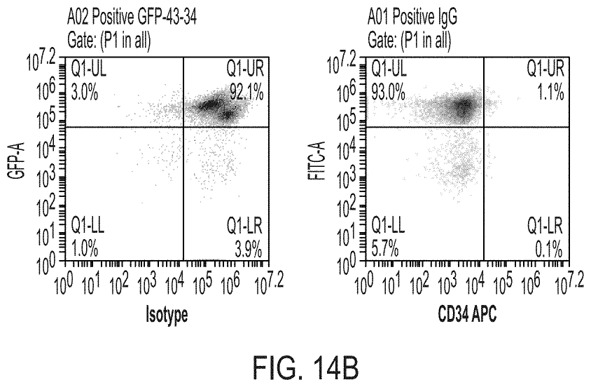

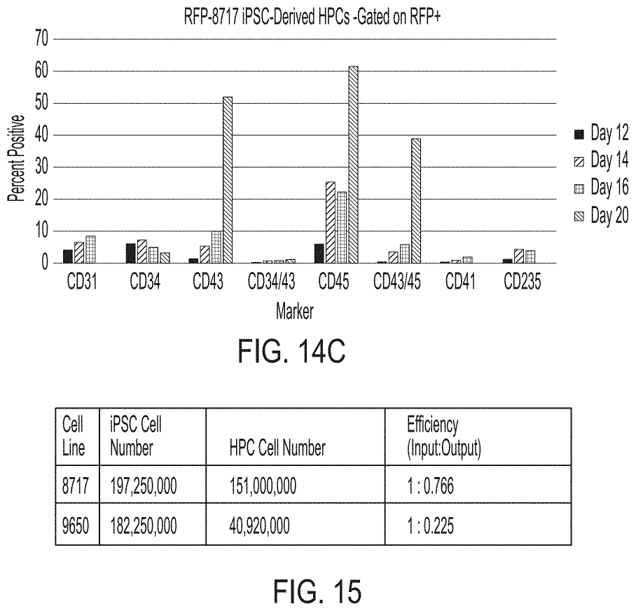

Generation of hematopoietic progenitor cells (HPCs) from GFP engineered 9650 and RFP engineered 8717 iPSCs: GFP engineered 9650 and RFP engineered 8717 iPSCs were maintained on Matrigel or Vitronectin in the presence of E8 were adapted to hypoxia for at least 5-10 passages. Cells were split from sub confluent iPSCs and plated at a density of 0.25-0.5 million cells per ml into a spinner flask in the presence of Serum Free Defined (SFD) media supplemented with 5 uM blebbistatin or 1 uM H1152. 24 hrs post plating SFD media supplemented with 50 ng/ml of BMP4, VEGF and FGF2 was exchanged. On the fifth day of the differentiation process the cells were placed in media containing 50 ng/ml Flt-3 Ligand, SCF, TP0, IL3 and IL6 with 10 U/ml of heparin. The cells were fed every 48 hrs throughout the differentiation process. The entire process was performed under hypoxic conditions. Purity of HPCs was determined by the quantification of CD4 and CD34 expression. The process outline is illustrated in FIG. 14. HPCs were purified further by magnetic sorting using CD34 antibody.

HPC purity was assessed starting at Day 12, and continued until CD34 expression reached >20%, as outlined in FIG. 14A. The differentiating HPC cultures retained the expression of GFP FIG. 14B. CD34⁺ MACS purification of line 9650 was performed on Day 15. RFP engineered 8717 revealed a lower efficiency of generating HPCs. Nevertheless, the cultures retained expression of RFP throughout the differentiation process. On Day 17, half of the culture was digested and plated for microglia differentiation, and the other half maintained as aggregates for macrophage differentiation. Efficiency of the process for both lines can be seen in FIG. 15.

TABLE 8
Exemplary formulation of Serum Free Defined Media (SFD),
EB#1 and MK#5 for generating HPCs from iPSCs.

| SFD | | | SFD EB#1 Medium | | SFD MK#5 Medium | |
|---|---|---|---|---|---|---|
| IMDM | 75% | | SFD | 100% | SFD | 50 ng/mL |
| Ham’s F12 | 25% | | BMP4 | 50 ng/mL | Flt-3 | 50 ng/mL |
| N2 (CTS) | 0.5% | | FGF-b | 50 ng/mL | SCF | 50 ng/mL |
| B27 w/o RA | 1% | | VEGF | 50 ng/mL | TPO | 50 ng/mL |
| (CTS) | | | | | | |
| HSA | 0.05% | | | | IL-3 | 50 ng/mL |
| MTG | 450 | μM | | | IL-6 | 50 ng/mL |
| Ascorbic | 50 | μg/mL | | | Heparin | 10 U/mL |

| | |
|---|---|
| Acid | |
| Pen/Strep | 1% |
| GlutaMAX | 1% |

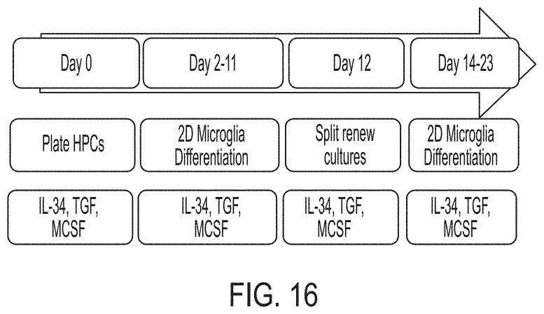

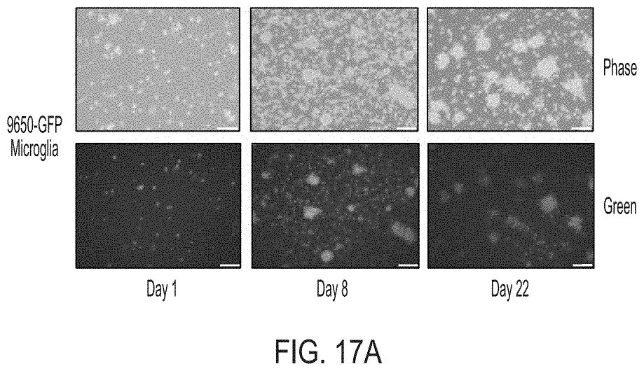

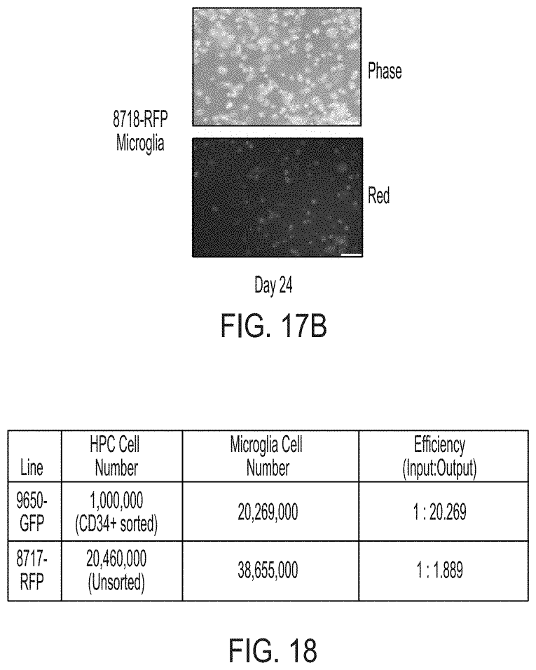

Generation of Microglia: Purified HPCs were placed in microglia differentiation media (MDM) under normoxic conditions. The cultures were fed using 2X MDM every 48 hours, with the differentiation process ending after 23 days. This process is outlined in FIG. 16. Morphology and fluorescence of the cells throughout the microglia differentiation process can be observed in FIGS. 17A and 17B. The efficiency of the process from HPC to microglia can be seen in FIG. 18.

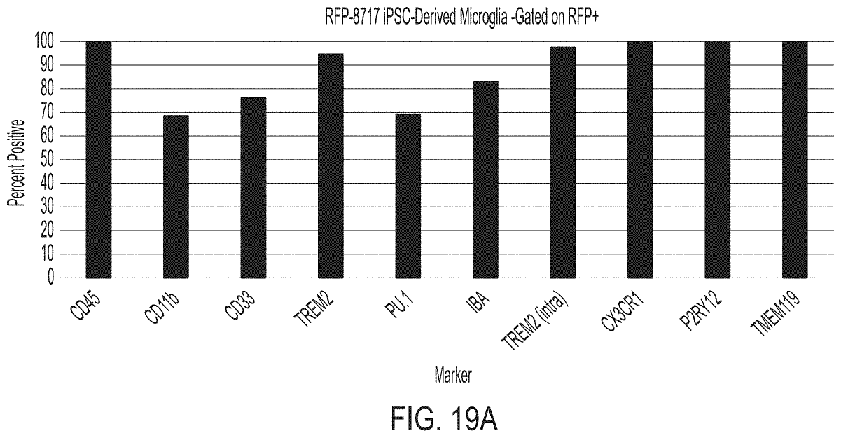

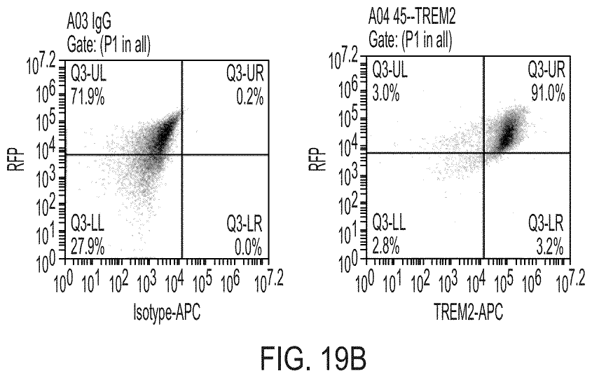

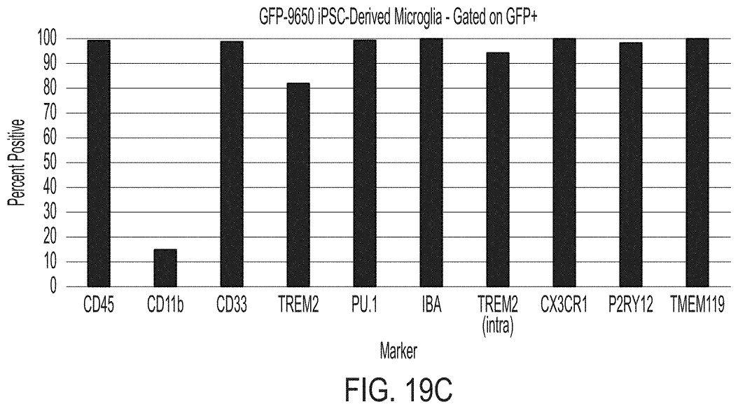

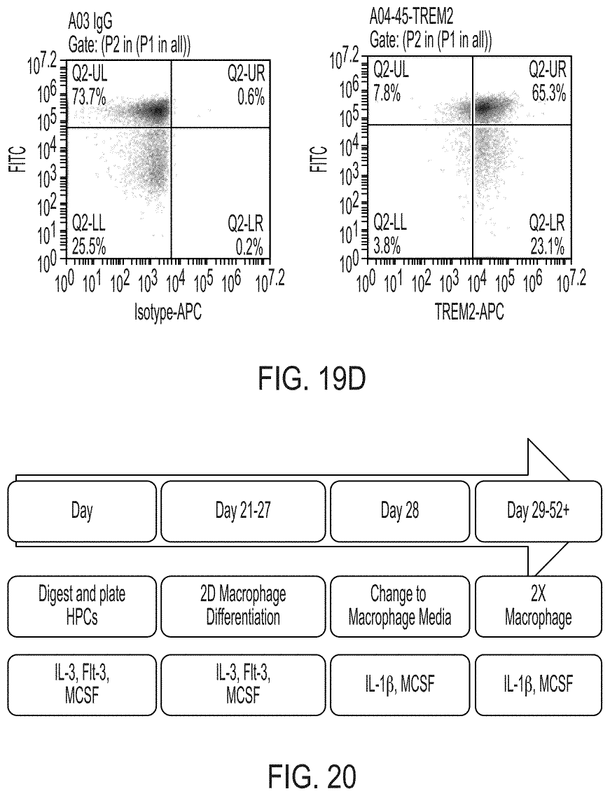

End-stage microglia cultures were assessed for purity. Cell surface expression of CD45, CD33, TREM2, and CD11 b, as well as intracellular expression of PU.1, IBA, P2RY12, TREM2, CX3CR1 and TMEM119 by flow cytometry (FIGS. 19A and 19B).

TABLE 9
Microglia differentiation medium.
Microglia Differentiation Medium-MDM

| Material | Supplier/Catalog # | Final Conc. | |
|---|---|---|---|
| DMEM/F-12, HEPES, no phenol red | ThermoFisher/11039021 | 94% | |
| N2 | ThermoFisher/17502048 | 0.5% | |
| B27 with RA | ThermoFisher/17504044 | 1% | |
| 10% BSA (in PBS) | Sigma/A1470 | 0.5% | |
| MTG (11.5M) | Sigma/M6145 | 450 | uM |
| Ascorbic Acid (20 mg/mL) | Wako/013-19641 | 50 | ug/mL |
| Pen/Strep | ThermoFisher/15140 | 1% | |
| GlutaMAX | ThermoFisher/35050 | 1% | |
| NEAA | ThermoFisher/11140050 | 1% | |
| ITS-G (100x) | ThermoFisher/41400045 | 1% | |
| Human Insulin | Sigma/19278 | 5 | ug/mL |
| MCSF (100 ug/mL) | Peprotech/300-25 | 25 | ng/mL |
| TGF-β1 (100 ug/mL) | R&D Systems/240-B | 50 | ng/mL |
| IL-34 (100 ug/mL) | Peprotech/200-34 | 100 | ng/mL |

TABLE 10
Microglia differentiation medium 2X.
Microglia Differentiation Medium-2X-MDM

| Material | Supplier/Catalog # | Final Conc. | |
|---|---|---|---|
| DMEM/F-12, HEPES, no phenol red | ThermoFisher/11039021 | 94% | |
| N2 | ThermoFisher/17502048 | 0.5% | |
| B27 with RA | ThermoFisher/17504044 | 1% | |
| 10% BSA (in PBS) | Sigma/A1470 | 0.5% | |
| MTG (11.5M) | Sigma/M6145 | 450 | uM |
| Ascorbic Acid (20 mg/mL) | Wako/013-19641 | 50 | ug/mL |
| Pen/Strep | ThermoFisher/15140 | 1% | |
| GlutaMAX | ThermoFisher/35050 | 1% | |
| NEAA | ThermoFisher/11140050 | 1% | |
| ITS-G (100x) | ThermoFisher/41400045 | 1% | |
| Human Insulin | Sigma/19278 | 5 | ug/mL |
| MCSF (100 ug/mL) | Peprotech/300-25 | 50 | ng/mL |
| TGF-β1 (100 ug/mL) | R&D Systems/240-B | 100 | ng/mL |
| IL-34 (100 ug/mL) | Peprotech/200-34 | 200 | ng/mL |

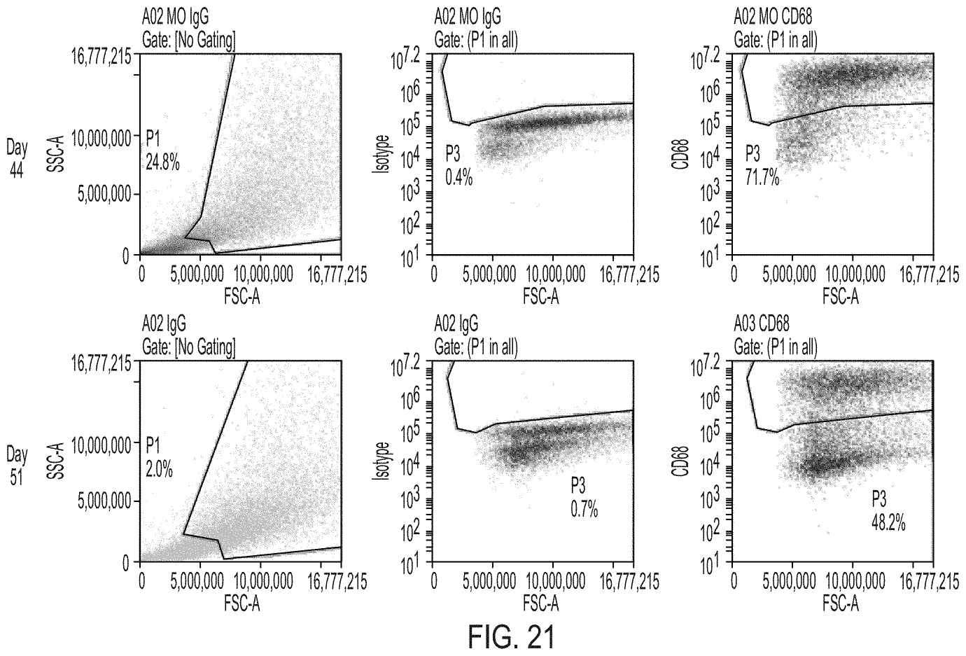

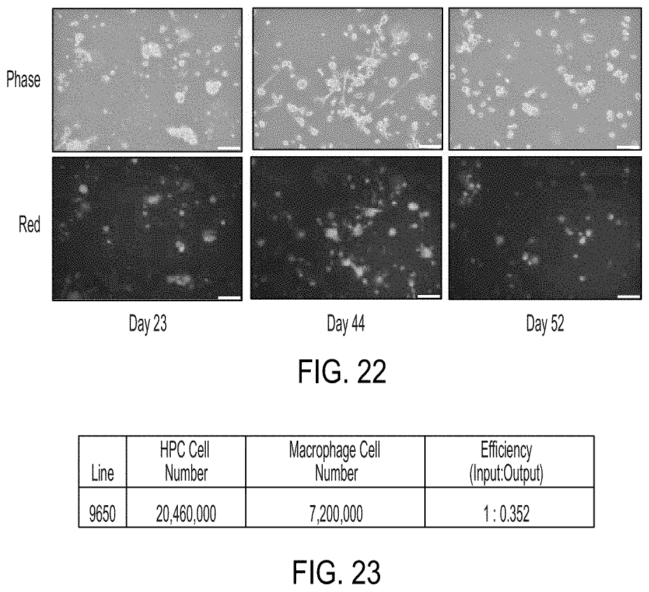

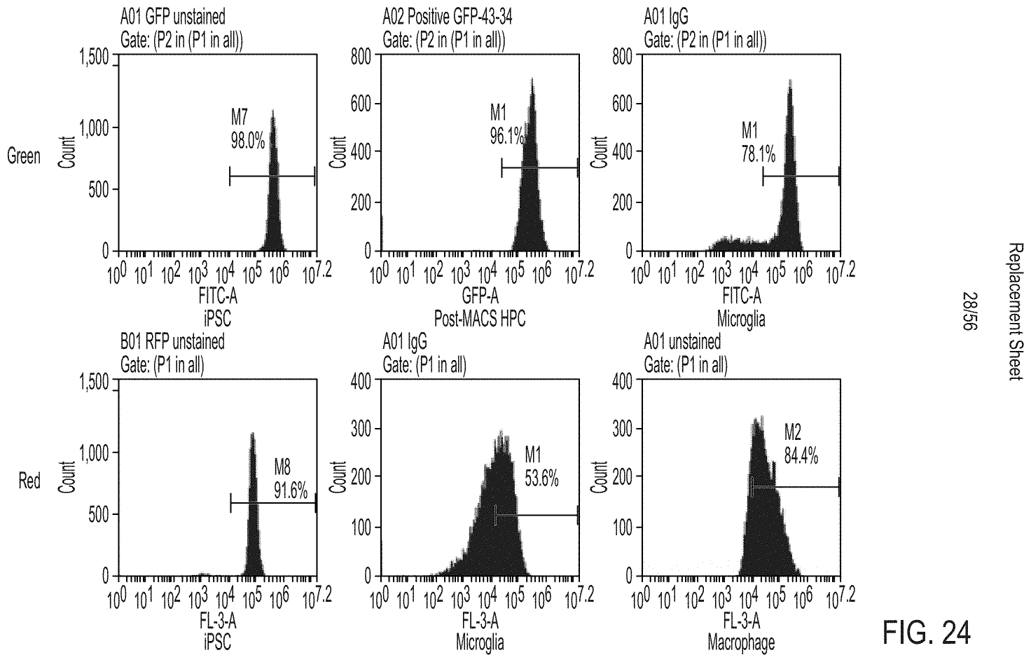

Generation of macrophages: Macrophage differentiation was initiated with line 8717-RFP on Day 17 of HPC differentiation. An outline of the macrophage process from HPCs is outlined in FIG. 20. Media compilations for this part of the differentiation are described in Table 11. On Day 20, the aggregates were digested and plated down in CMP Media. At this point the culture was changed to a normoxic environment. After one week, the culture was changed to Macrophage Medium, and fed 2X Macrophage Medium every 4 days thereafter. CD68 purity was assessed at Days 44 and 51 and is shown in FIG. 21. Cells were harvested and cryopreserved on Day 52. Morphology and fluorescence of the cells can be seen in FIG. 22. Efficiency of the process from HPC to Macrophage is described in FIG. 23. Fluorescence intensity as measured by flow cytometry from iPSCs to HPCs, microglia and macrophages is demonstrated in FIG. 24.

TABLE 11
Media formulations
CMP Media

| SFD | | |
|---|---|---|
| M-CSF | 50 | ng/mL |
| IL-3 | 50 | ng/mL |
| Flt-3 | 50 | ng/mL |

Macrophage Media

| SFD | | |
|---|---|---|
| M-CSF | 20 | ng/mL |
| IL-1β | 10 | ng/mL |
| Excyte | 0.3% | |

2X Macrophage IV Media

| SFD | | |
|---|---|---|
| M-CSF | 40 | ng/mL |
| IL-1β | 20 | ng/mL |
| Excyte | 0.3% | |

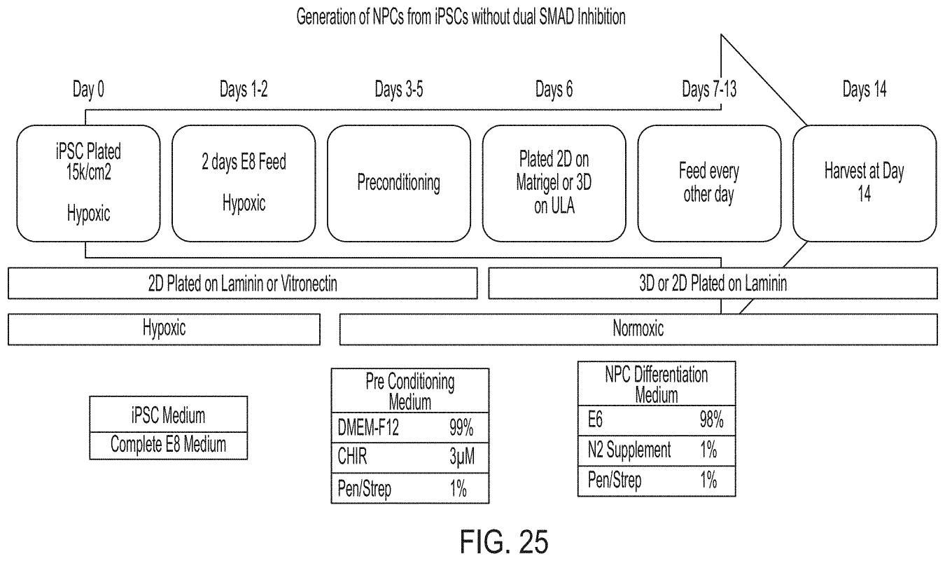

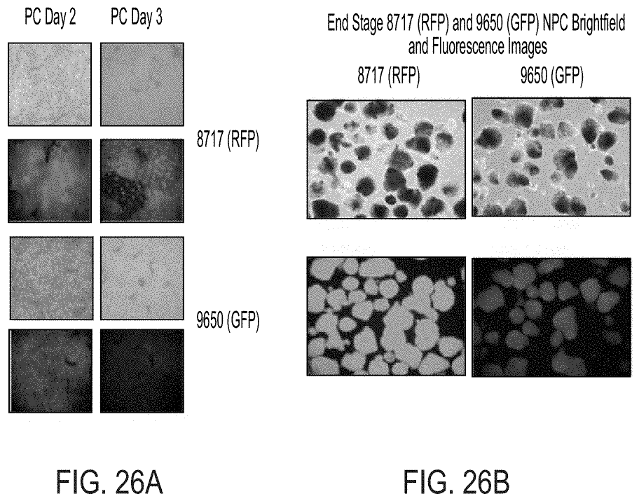

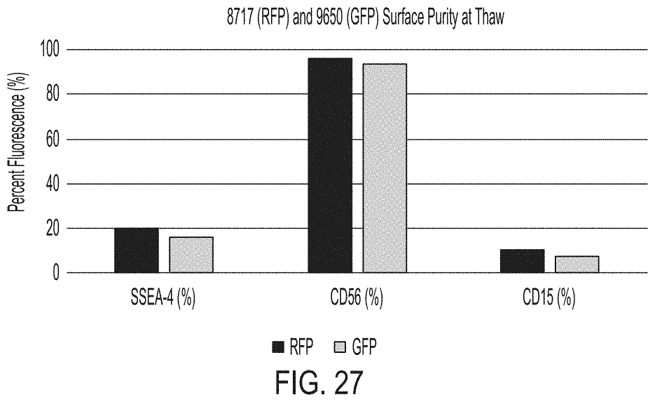

Generation of neural precursor nells (NPCs) from 8717-RFP and 9650-GFP engineered iPSC: Neural progenitor cells (NPCs) are self-renewing progenitors with the ability to generate neurons and glia (Breunig et al., 2011). There are many established protocols with varying efficiencies for generating NPCs from primary neural cells and iPSCs (Shi et al., 2012a, Shi et al., 2012b). Most of the recent protocols rely on the inhibition of the SMAD signaling pathway. The present methods describe a simple protocol to generate NPCs across different iPSCs lines utilizing the spontaneous drift of iPSC towards ectoderm without using the dual SMAD inhibition pathway. A schematic description of the method to generate neural precursor cells (NPCs) from iPSCs without using dual SMAD inhibition. The various steps involved, and the composition of medias used is described in FIG. 25. Briefly, episomally reprogrammed iPSC lines, 8717-RFP and 9650-GFP were maintained on Matrigel/Laminin/Vitronectin coated plates and E8 media. The iPSCs were maintained under hypoxic conditions before the onset of differentiations to generate NPCs. To initiate neural precursor differentiation, iPSCs were harvested and seeded at 15 K/cm² on Matrigel, Laminin or Vitronectin plates using E8 media in the presence of rock inhibitor. The cells were placed in fresh E8 media for the next 48 hours in the absence of rock inhibitor. The next step involved the preconditioning step that involved placing iPSC cultures in DMEMF12 media supplemented with 3 μM CHIR for 72 hours with a daily change in media under normoxic conditions. Cells were harvested at the end of the preconditioning step and either replated back in a 2D format on Matrigel, Laminin or Vitronectin plates at 30 K/cm² or generated 3D aggregates using Ultra low Attachment (ULA) plates or spinner flasks at a density of 0.3 million cells per ml in the presence of a rock inhibitor. The cultures were fed every other day with E6 media supplemented with N₂ for the next 8 days under normoxic conditions. The retention of GFP and RFP fluorescence throughout the differentiation process is captured in FIG. 26. On day 14 of differentiation the cultures were harvested and individualized using TrypLE. The cells were stained for the presence of SSEA4, CD56, CD15 by cell surface staining. The quantification of purity of NPCs is depicted in FIG. 27. CD56 was used as the marker for NPCs derived by this method. The cells were cryopreserved using CS10 and they retained purity and proliferation potential post thaw.

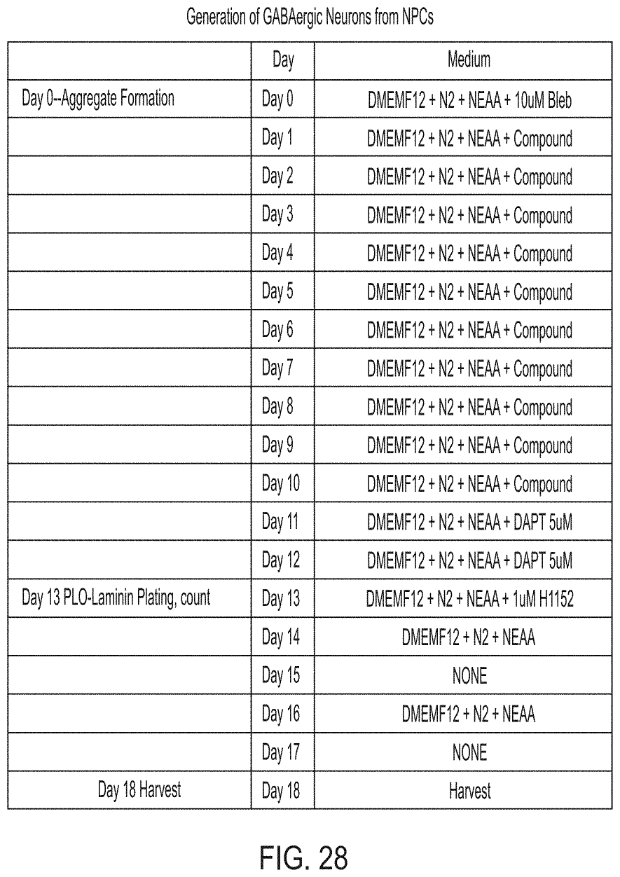

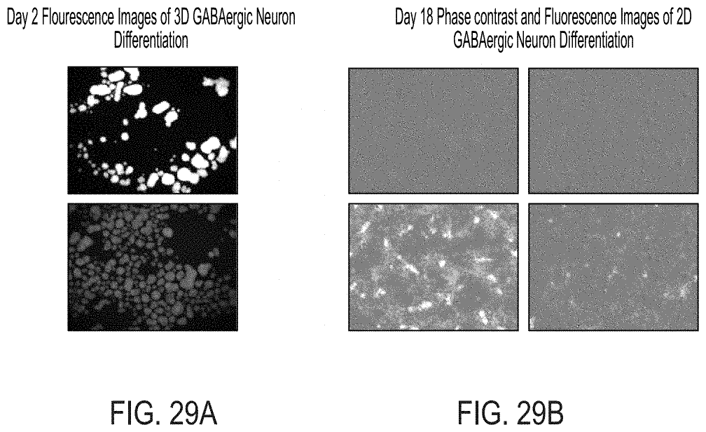

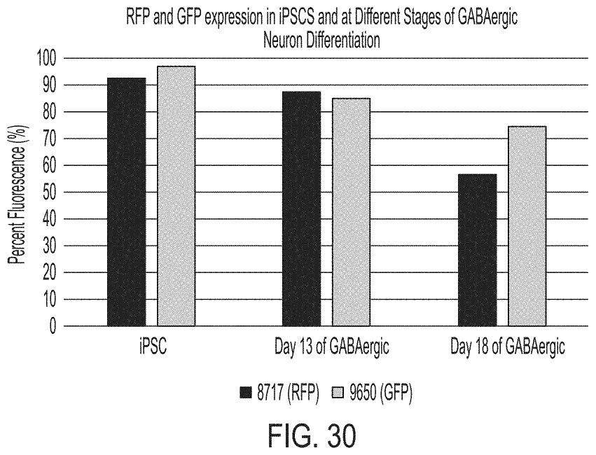

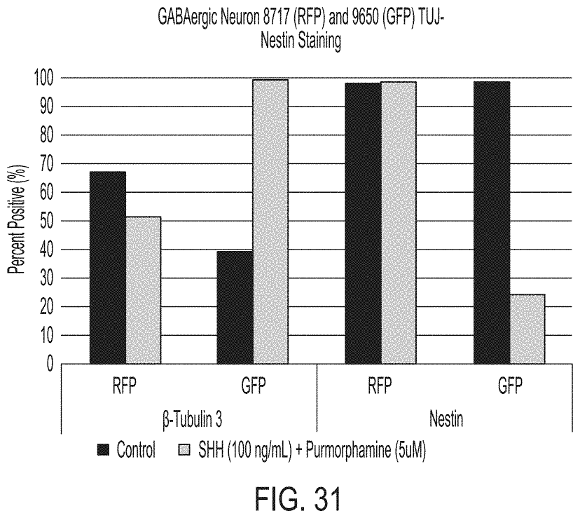

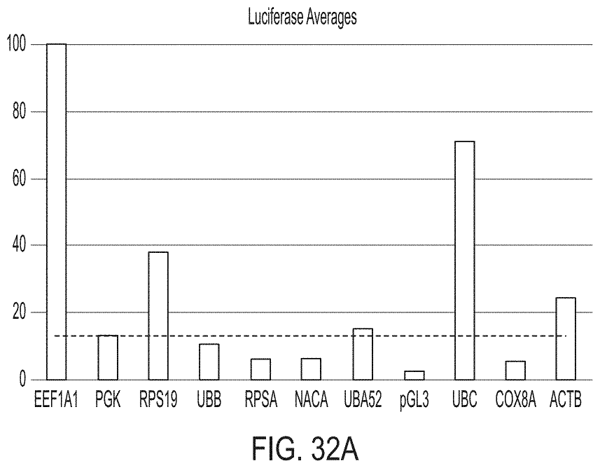

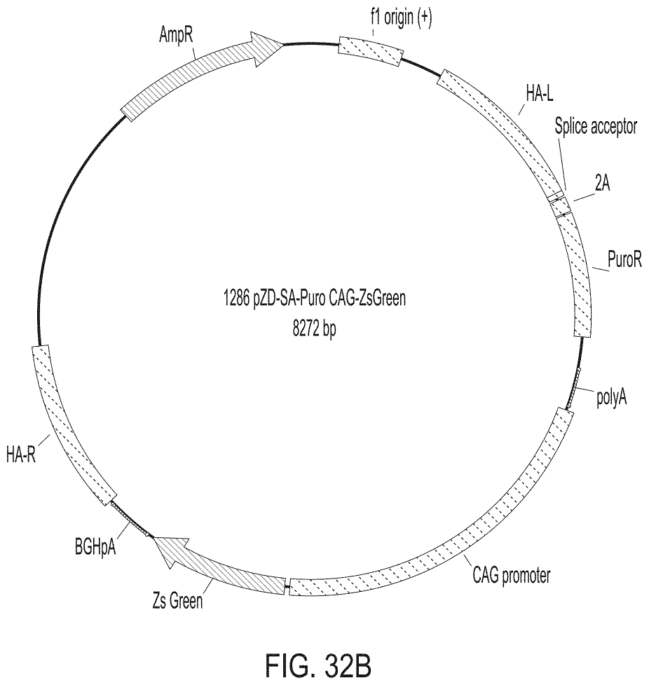

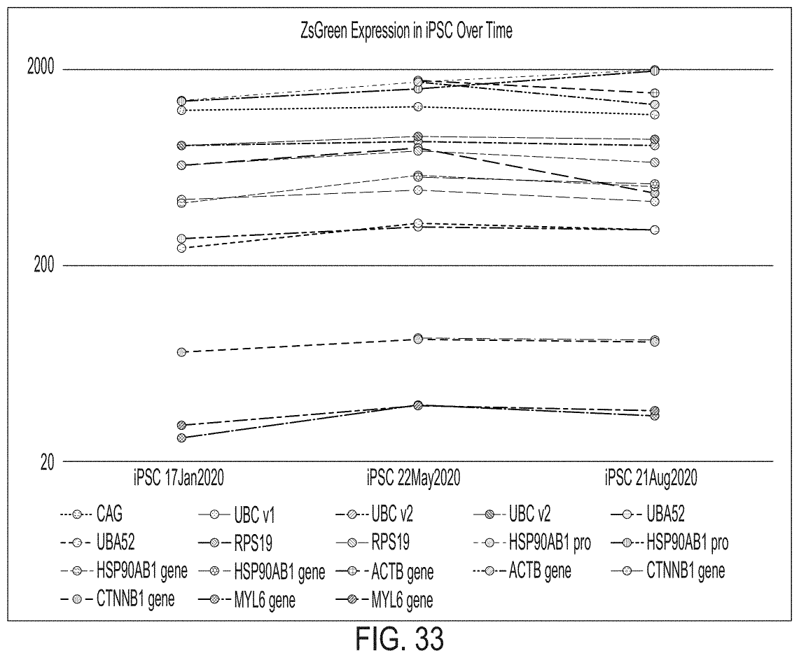

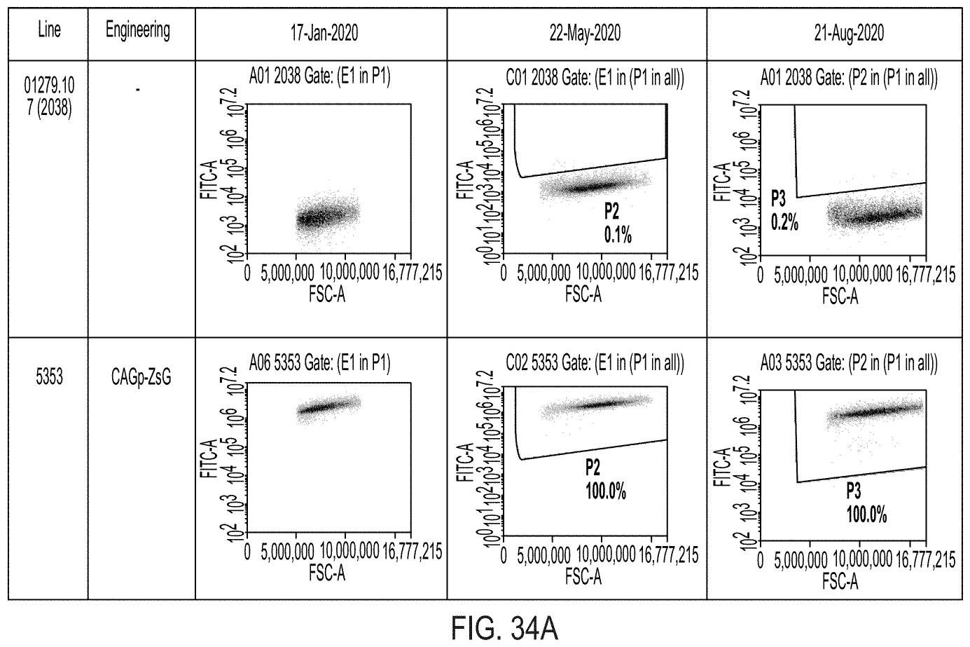

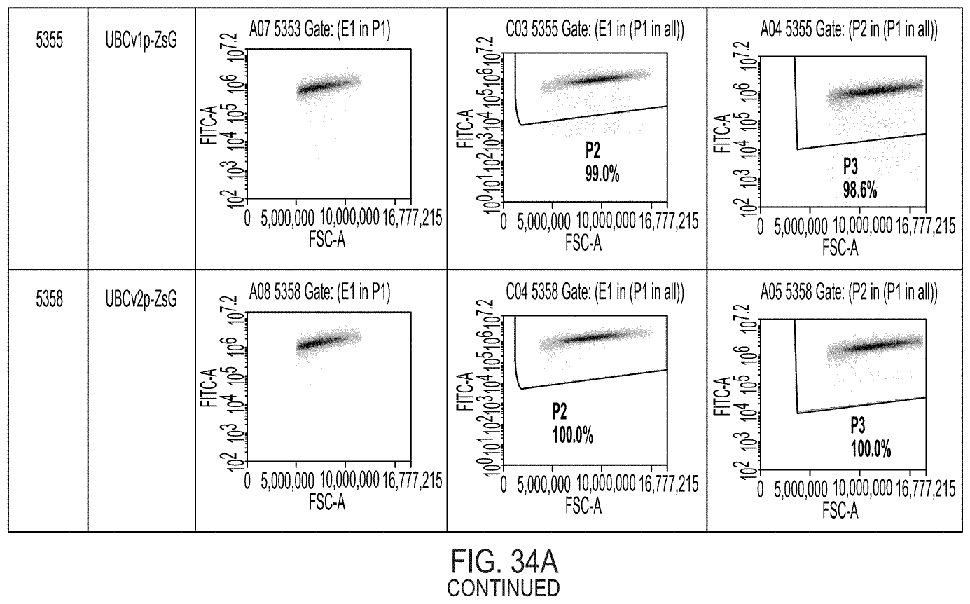

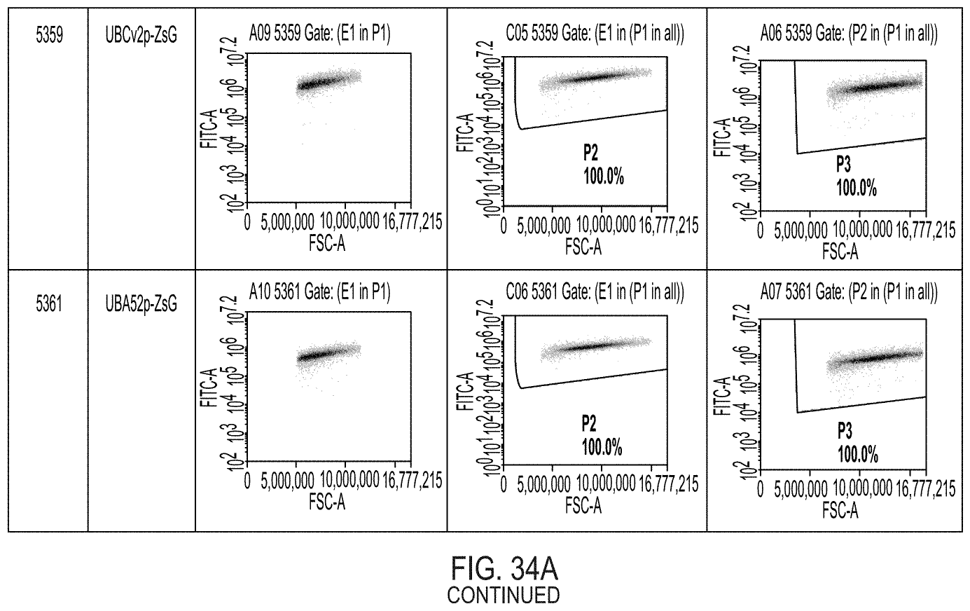

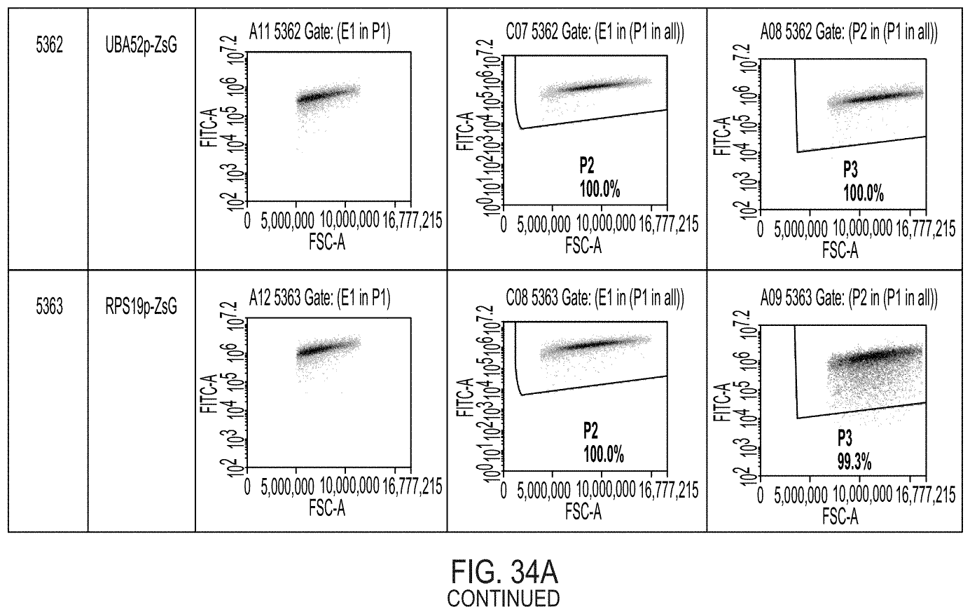

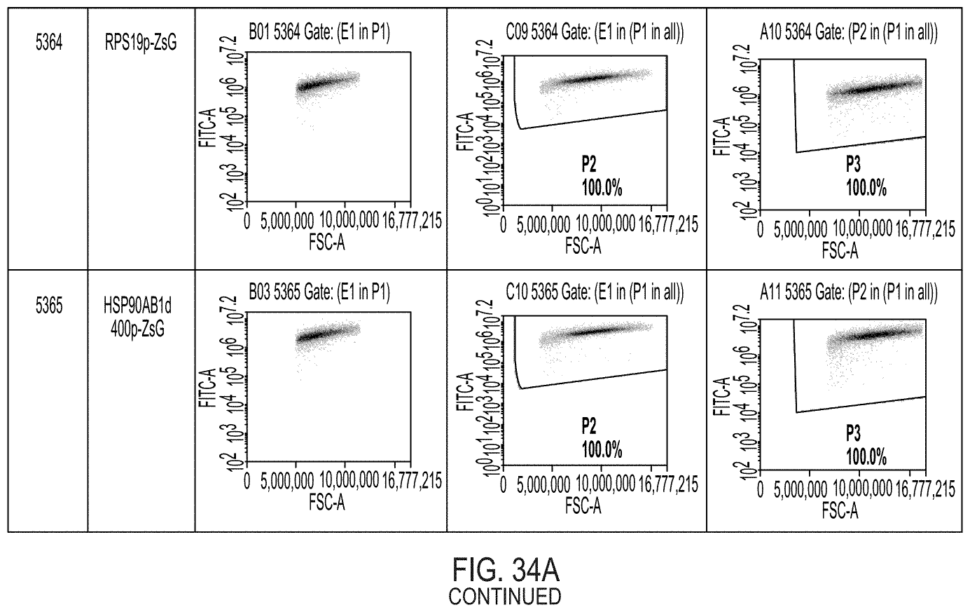

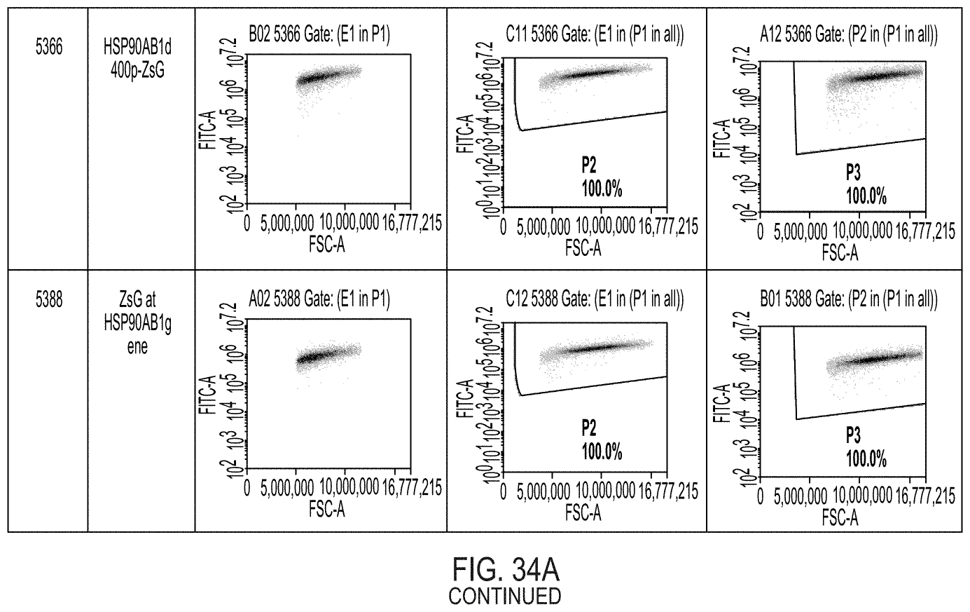

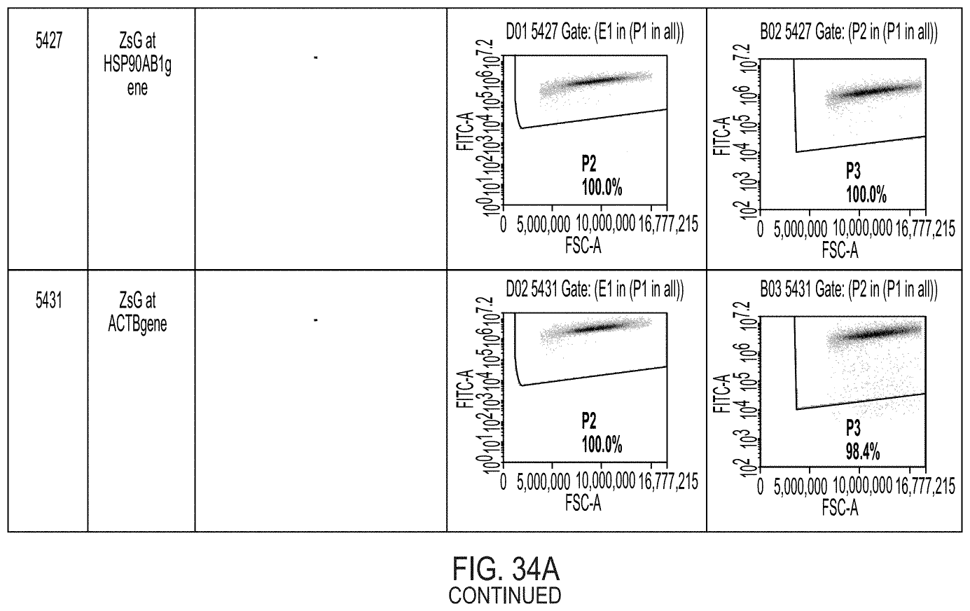

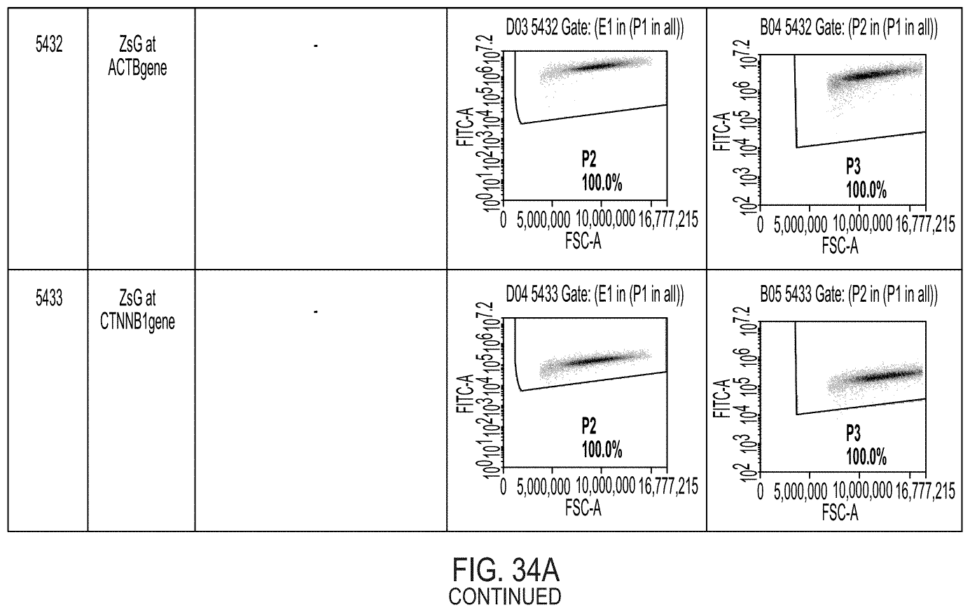

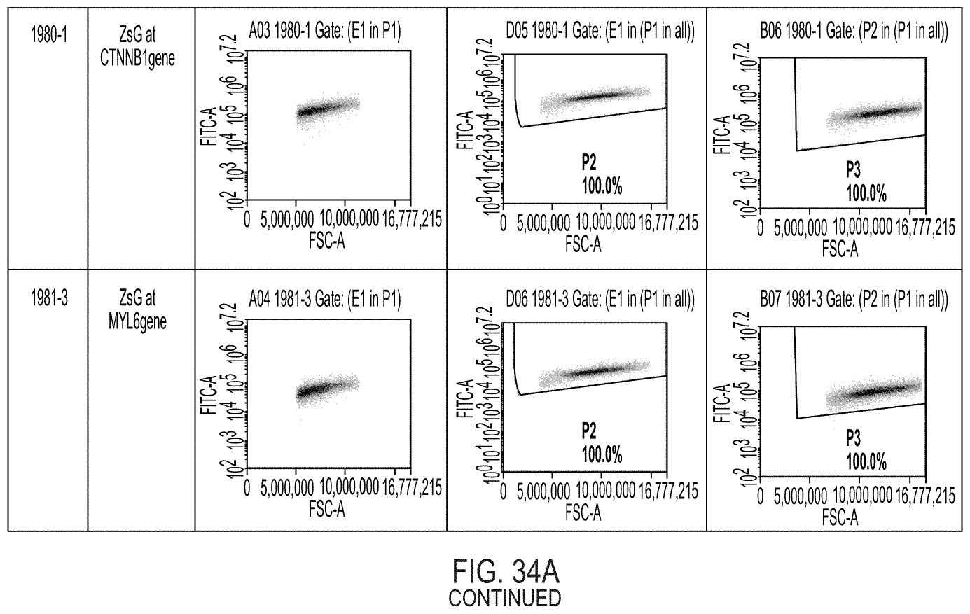

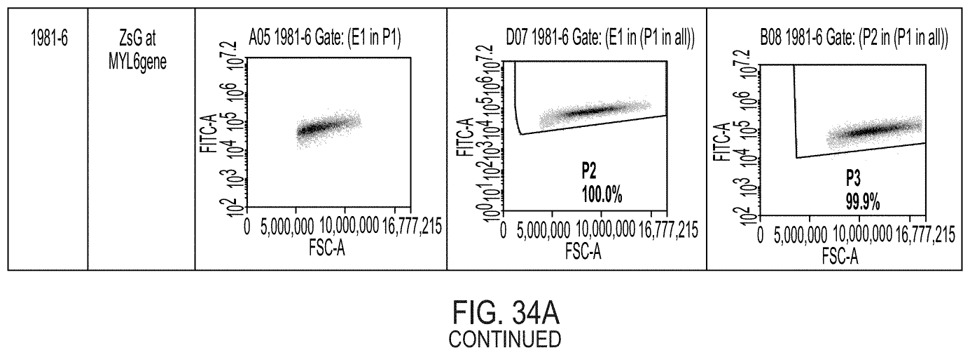

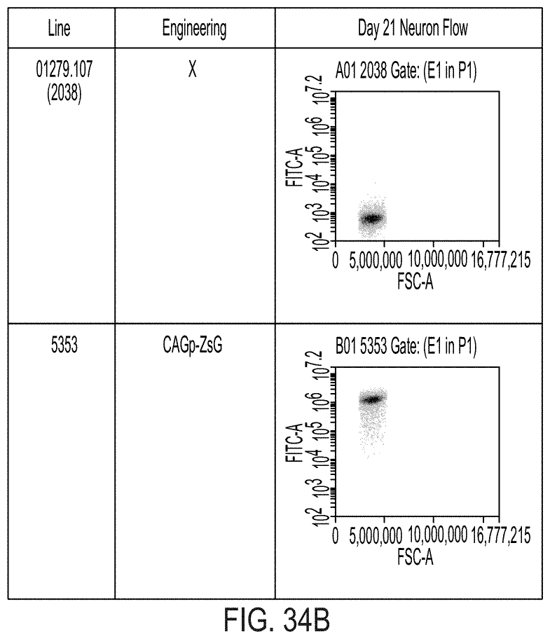

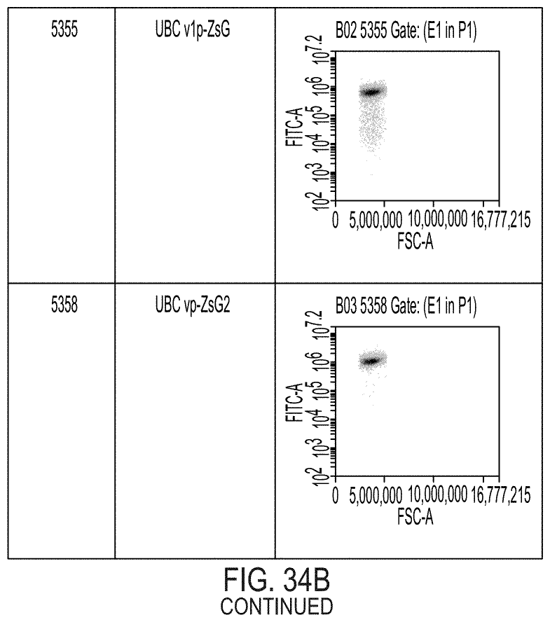

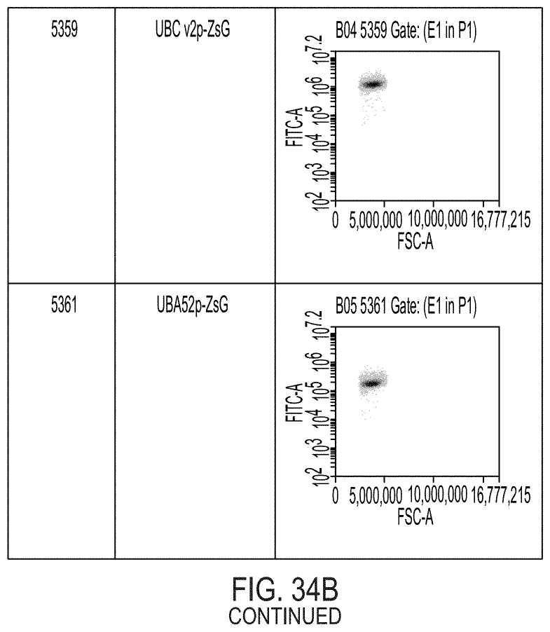

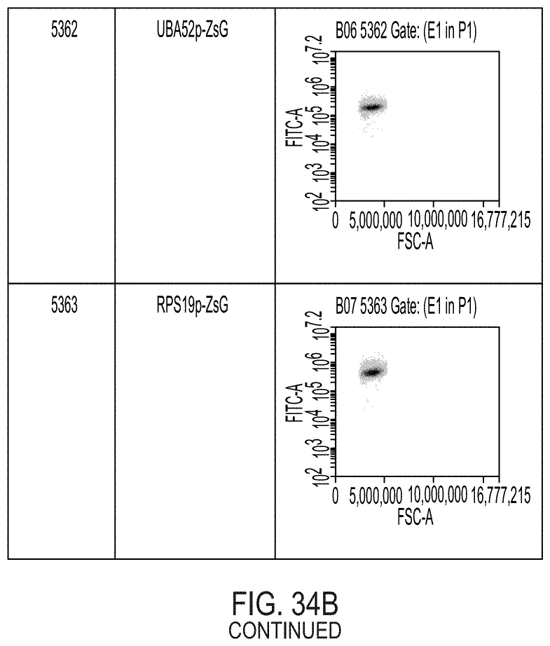

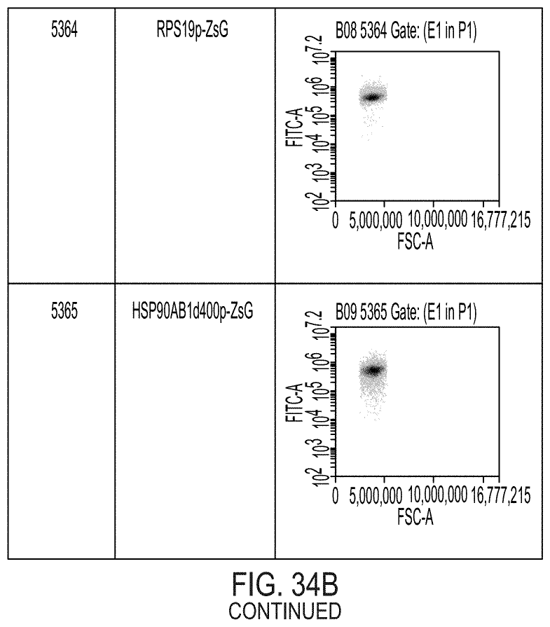

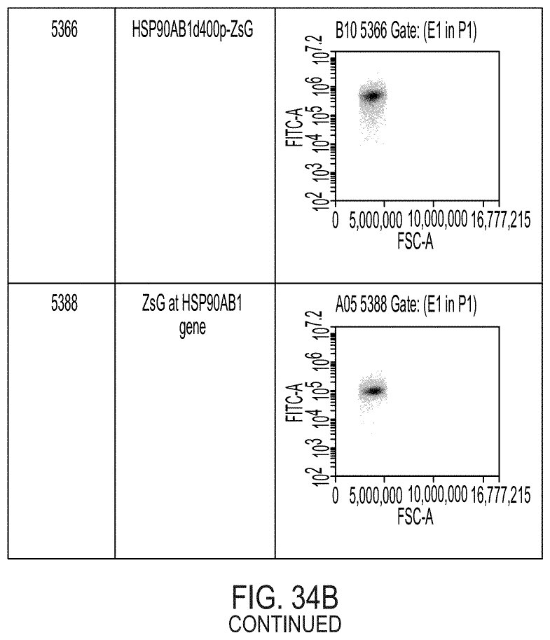

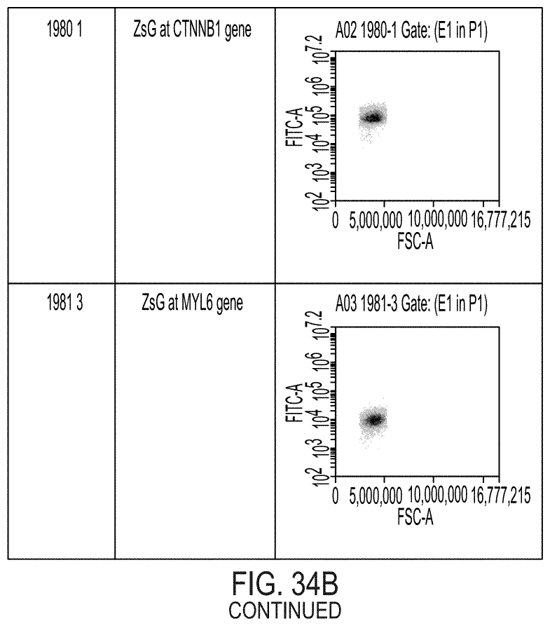

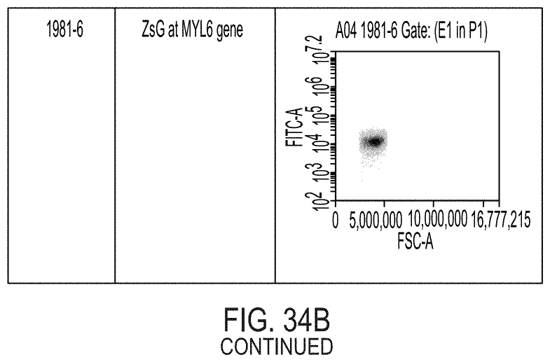

Generation of GABAergic neurons from neural precursor cells: The potency of NPCs was tested by thawing NPCs and placing the cells in a differentiation pathway outlined in FIG. 28. Briefly, NPCs were placed in a downstream differentiation protocol to generate GABAergic neurons. NPCs were thawed and seeded at 0.3e6/mL in the of DMEM/F12 supplemented with N₂ and NEAA, in the presence of 10 μM Blebbistatin for 24 hours to form aggregates. Cultures were given a complete media exchange every day with DMEM/F12 supplemented with N₂ and NEAA, with Sonic Hedgehog Signaling Molecule (SHH) and Purmorphamine at 100 ng/mL and 1.5 μM respectively, for 10 days. For the next 48 hours cultures were fed DMEM/F12 supplemented with N2, NEAA, and 5 μM DAPT prior to being plated at 200,000/cm² onto PLO-Laminin coated plates using DMEM/F12, N2, NEAA, and 10 μM Blebbistatin for 24 hours. Cultures were fed DMEM/F12, N2, NEAA, and 5 μM DAPT every subsequent day and harvested 5 days post plating. The retention of fluorescence in emerging cultures of GABA neurons is depicted in FIG. 29. The quantification of the intensity of GFP and RFP from the iPSC stage to day 18 GABA neuron differentiation is captured in FIG. 30. Finally, the purity of ends stage GABA neurons by quantification of Nestin and β-Tubulin 3 purity is depicted in FIG. 31. These cell differentiations showed that the CpG optimized iPSCs may be differentiated to multiple cell types including, but not limited to, those described above.

### Example 3—Promoters for Stable Expression

Stable transgene expression in iPSC lines across time and post-differentiation has been challenging to achieve. Many promoters show silencing or variable expression, and previous studies have shown this for promoters such as PGK and EEF1A1. The following studies were carried out to identify either promoters or tag-able gene loci that could be used to provide stable expression in both iPSC and differentiated cell types. It is often during the process of differentiation where DNA methylation changes significantly and can affect expression; thus, the best promoters need to be active in both dividing cells and quiescent cells that have minimal cell division (such as mature, fully differentiated cardiomyocytes).

Promoters cloned: The following promoters were identified as being likely candidates for constitutive expression in all cell types. Some were cloned from existing plasmids (CAG, PGK, UBC-version1, EEF1A1, ACTB). Other regions are newly generated (by PCR from genomic DNA or by synthesis) with the goal of identifying promoters that would provide stable expression in both iPSC and differentiated cells. The new promoters include RPS19, UBA52, HSP90AB1, an enlarged region of UBC (version 2), UBB, RPSA, NACA, and COX8A. Sequences were cloned into the pGL3 plasmid vector (replacing the SV40 promoter between MluI and Ncol restriction sites) to enable a comparison of promoter strength when driving the luciferase reporter gene.

TABLE 12
Promoter sequences

| PLASMID | PROMOTER SEQUENCE |
|---|---|
| 1948 PGK-pGL3 | ACGCGTATCCCGGCGCGCCCTACCGGGTAGGGGAGGCGCTTTTCCCAAGGCAGTCTGG |
| (SEQ ID NO: 1) | AGCATGCGCTTTAGCAGCCCCGCTGGGCACTTGGCGCTACACAAGTGGCCTCTGGCCT |
| | CGCACACATTCCACATCCACCGGTAGGCGCCAACCGGCTCCGTTCTTTGGTGGCCCCT |
| | TCGCGCCACCTTCTACTCCTCCCCTAGTCAGGAAGTTCCCCCCCGCCCCGCAGCTCGC |
| | GTCGTGCAGGACGTGACAAATGGAAGTAGCACGTCTCACTAGTCTCGTGCAGATGGAC |
| | AGCACCGCTGAGCAATGGAAGCGGGTAGGCCTTTGGGGCAGCGGCCAATAGCAGCTTT |
| | GGCTCCTTCGCTTTCTGGGCTCAGAGGCTGGGAAGGGGTGGGTCCGGGGGCGGGCTCA |
| | GGGGCGGGCTCAGGGGCGGGGCGGGCGCCCGAAGGTCCTCCGGAAGCCCGGCATTCTG |
| | CACGCTTCAAAAGCGCACGTCTGCCGCGCTGTTCTCCTCTTCCTCATCTCCGGGCCTT |
| | TCGACCTGCAGCCGAGATCTAGTACTAGTGGGCCACCATGG |
| 1949 RPS19-pGL3 | ACGCGTTAACATAATTTTATTGGACCACGTTATTTAGTTGTTGGGCCTTGTAAGGATT |
| (SEQ ID NO: 2) | AAATGAGATCATGCATGTAACACTACAGTAACAGCCACATGATAAATGTCCAAATAAT |
| | ATTTACCTGTGCCTGGCACAGAGCAGGCACTCAAAAAATATTTTTTAGAGCATGTGAC |
| | GCGCCATGAACCAGAGGAGCCACTTTAAATGGACAAACGGGGATCTCATTTTTTTTTT |
| | TTTTTTTAATGGTGAGACAAGCCCTGAGAAGGCAAATGGACTGCCTAAAGCTACACAG |
| | GTCAGCGGGGCAGGTAAATCTAAATTGGCAAAGTAAGGGCTGAGCGAACAGACTCCGA |
| | CACTAGAAGGCAGAGCTGAACTAACTTCTGCTAAGGTCCCGCCTCTGCCGCTTTGTCC |
| | CGCCCTTATCTTCTCCCCTCCTCCAGCGCCTCATTCCCTTTTCGCTCGCCCCGGCCGT |
| | GCTGAAGCAACTTCCGCCCTGAGAAGGGTGGGGCTTCCGTCTCCCGCTCTCGCGACTC |
| | CTGGCGGTGAAGGACGGAAGATGATAGCCACATTTCTTCCTCGCCCTTCCCCTAGGTT |
| | CCCTGTCACAGTTCCGCCCTTACTACTCCCACTTCCGGCCAGGGAACAGCCACTTCCA |
| | CCCGGAAAAGGGGTTGTTCCGCCGTGGGGCGCCAGCTGTGGCCCACCCATCCTGCCCC |
| | GTACTTTCGCCATCATAGTATTCTCCACCACTGTTCCTTCCAGCCACGAACGACGCAA |
| | ACGAAGCCAAGTTCCCCCAGCTCCGAACAGGAGCTCTCTATCCTCTCTCTATTACACT |
| | CCGGGAGAAGGAAACGCGGGAGGAAACCCAGGCCTCCACGCGCGACCCCTTGGCCCTC |
| | CCCTTTACCTCTCCACCCCTCACTAGACACCCTCCCCTCTAGGCGGGGACGAACTTTC |
| | GCCCTGAGAGAGGCGGAGCCTCAGCGTCTACCCTCGCTCTCGCGAGCTTTCGGAACTC |
| | TCGCGAGACCCTACGCCCGACTTGTGCGCCCGGGAAACCCCGTCGTTCCCTTTCCCCT |
| | GGCTGGCAGCGCGGAGGCCGCACGGTAAGCGGGGGCTCCGAGCTGGACCGGGCGCGAG |
| | GTGGCAGGGCCGGACGCCGAAGCCTCAGAGCGCGTGCCTGAGGGCCCCGAGGCGCCCG |
| | GCGCGGGCCCGTCCCGCCCCCTAGAGCCGCGGCCACGTGCGAGCGGCAGGCCCGGACA |
| | TGCCCGGTCAGCGCCGTCCGGGAACCGAGCGTGGGCCCCGGGGGGCAGCGGCGGGGTG |
| | CGTGGGGCGTCCGGAGTCCCGGGGCTGGGGAGTGGGGTCGCGCAGGATCCTCACACGC |
| | AGGGGCCGGGCTCTGTTAGTGCGATCCAGAGAGGCCGTGGGCGTCGGTGAGCTCCTTC |
| | AGACCCGCAGGAGCCGGAGCCCGGCGTTGAAGGGGCCGTGGGAAGTAACGGGGGGTAC |
| | CACGGTTTAGGATGCGCTGGAGCGAAAGGATTGGGGTGGGGTCCGTGCTCTTGGCAGT |
| | CGTCTCTGCCAGGCCTGTGTTCACATGCTTGACTTTCTCCCTCAGGTACCTGGAGTTA |
| | CTGTAAAAGAGCCACCATGG |
| 1950 UBA52-pGL3 | ACGCGTGCTAGCCCGGGCTCGAGATCTCTCGGGAGGCTGAGGCAGGAGAATTGCTTGA |
| (SEQ ID NO: 3) | ACCCAGGAGGCGGAGGTTGCGGTGAGCCGAGATCGCGCCATTGCACTACAGCCTGGGC |
| | AACGAGAGCGAAACTCCGTCTCAAAAAAAAAAAAAAAAAAAATCCTGAGTCCCGCTTG |
| | ACACCTTTTGTCAGGCACCACCACCTTTCTGGGCGAATGCGGTAGTACCGTCTGCTCT |
| | CCCTGCTGCTGTCCTGAAATCCATTCAGGCACAGCGGCCGAGAGCTTTATAATAACCG |
| | ATTCCAGGTGTTAGGTGCTTTCCCAGCCCCGACTCCTGCGTCCTGGACCCGCAGTCCT |
| | CTGCTTAATACCTTTGCTTTATTAGAAAACATTCTCCTCTACTCCGTTCAGCTATTCG |
| | CTGAGGGCCCGCCAACCGCCAGCGGTTGTCAGTGGCCTAGAGGCAGCGGACGCAAACA |
| | CGGGGAGAGGTGCAATCGTCTCAAGTGACTCGGCGGGCGGGGCCCACAACCGGAAGCG |
| | GGTGGGCGACCTTCACCCACGTGCGCTGCGGCTTCGTTCGCCAGCATCCAAGATGGCG |
| | GCAGGGCGGGGCCCAAGGCGCGGCGCGAATTGTGACGCAGGCGTCCGGCGTGCTCCGT |
| | GCGCAAGCGCTTTCGGCGGCGATTAGGTGGTTTCCGGTTCCGCTATCTTCTTTTTCTT |
| | CAGCGAGGCGGCCGAGCTGGTTGGTGGCGGCGGTCGTGCGGGTTCGCGCCGGGCCGAG |
| | AGCGGGTTGGGGGCTGCGGGAGGCTGCAGGGGCCTGGGCGGCAGAAGAGGCGGCCCTG |
| | AGCTGGCTCATGCGGGCCAGTCTCGGCAGGGTGGCTGGGCAGGGCTCGCGAGGCCACG |
| | GCTCGGAGCCCAGACCGGGGCCCAGGAGGCGAGCGCCGTTTTGGAGAGGAGCCTGCCT |
| | GCTCTGCCTGCCAGCGTGACCCCACGAGGCCTCGGGCGGGAAGAGGTCCTCGGGGCAG |
| | ATCCGAGTTAATGAGAGAGGGGTATTGAGCGTGTAGCGTTAACTCTGCCAGTCACTGC |
| | GTCAGTCGCTTTGGAAATACTAAATTTCTCGAGCTGAGTCTTCATACCTGGCTCCATT |
| | ACTACGTCTGTAAGGAGGAGCTGGTGGTAGTGTCTGCTTTTTAGACTTTTCTTTAGAC |
| | TATTTGTATTTTTTTCAGATGGAGTCTTGCTCTGTCGCCTAAGCTGGAGTTCAGTGGT |
| | GCGGTCTCGGCTCACTGCAATCTCCACCTCCCGGGCTCGAGCGATTCTTCTGCCTCAG |
| | CCTCCCGAGTAGCTGGGATTATAGGCGCCTGCCACCACGCCCAGTTGATTTTTGTAGT |
| | TTTAGTAGAGACGGAGTTTCACCATGTTAGCCAGGCTCATCTTGAACTCTTGACCTCA |
| | AATGATCCGTCTGCCTCGGCCTTCCAAAGTGCTGGGATTACAGGCATGAGCCCCTGCG |
| | CCCGGTCGATTCTTTGTCTTTTTAAGTCAACTTTTATATGTGAACAATGCTTGGCAGG |
| | TGGTTGGTAGATACTAAGTGATGTTCGTGGTTTGGGGTCAAGGCAAGAAGTGGGGTCT |
| | GGAGAGTTTTGGTGTAATTGAGAAGGAAGCTAAGAGTGTTGGGTGCTCCAGCTTGGAG |
| | TTAGAGAGGAGAGAGGCTGCGACAGGAAGGCATGTGTGTTGTAGGGGATGGCTTCCCA |
| | TCCAGGCTGGCAGCAGGAGCAGCCTGTGCAGATCAGGACCTGGCTGCCGTGGAAGAGG |
| | GTGGGACCGCCTTCAGGGAAGATGGATCTAGCAAGTTGAAGCCAAAGGGTACTTATTC |
| | CATCAGGAGATACTGACGAGTCCTTCCGCCGCTAAACCTAAGGAGAATAACCACAGTC |
| | TGTGTTCCTGAAGAGCACCCGTGCGGTCAGGAGGGTGGAGGACATGTGGTCTTTAGTT |
| | CCAGGACATGTTTAGACTACAGGCCAGGGTGTGTGAGAAGCCTAGCAGGGCCAGGCTT |
| | GGAGGAGTGAAAGGAAGACAGGTACTGGGGCAGGACCAGTTGGACTTGGTGCAGGCAA |
| | AGGGATAGCAACTGTGGTGTAGGCACCTGAGCTTGTGCTACTCAGGCATGCATTGCTC |
| | ACCAGTCTATCCTGCCTCCCTTCCTCCTGCAGACGCAACCATGG |
| 1951 HSP90AB1 | ACGCGTGCTGCCCTGCACTGGTTCCCAGAGACTCCCTCCTTCCCAGGTCCAAATGGCT |
| (del 400)-pGL3 | GCAGGAGCGAAGTGGGCGGAAAAAAAGCGAACCAGCTTGAGAAAGGGCTTGACGTGCC |
| (Contains a 417 bp | TGCGTAGGGAGGGCGCATGTCCCCGTGCTCCGTGTACGTGGCGGCCGCAGGGGCTAGA |
| deletion between | GGGGGGTCCCCCCCGCAGGTACTCCACTCTCAGTCTGCAAAAGTGTACGCCCGCAGAG |
| two Clal sites) | CCGCCCCAGGTGCCTGGGTGTTGTGTGATTGACGCGGGGAAGGAGGGGTCAGCCGATC |
| (SEQ ID NO: 4) | CCTCCCCAACCCTCCATCCCATCCCTGAGGATTGGGCTGGTACCCGCGTCTCTCGGAC |
| | AGGTCAGAGCGGGTCGCCGGGTGGGGTCGCTGCAAAAACCCTGCCCCGGCCGCAGCCG |
| | GAGAGGCGGAGCCTCGCGGGGAGGGGGCGGGACCGCCGAGACAGGCCTGGAAACTGCT |
| | GGAAATGCCGCAGTGCCGCCGCCGCGCCTTCCGCCGCATGTCGGCAAAGAGTCCCCGC |
| | CAGCCCCGGCCGGCGCCCTCCCCCTACGCTGAGCTGCCCCTCAGCGCGAACCCTCCGC |
| | CCTTCCTCTACTCCTGCGAGAGTCGGGATCTGGGGCTACCCAAGGTTGGGTCCCGAAT |
| | GCCAGTCCCTCTGTCGGGACGCGAGATGTGTAGGGCAGATGCTAGGAAGAAGATTGGG |
| | TCTGGGAGCGGTGGTCCGCGTGGTTAGCTGCCTCCGCTCTTTTTCGGTGTCCCCCCCA |
| | GTCCCGCCCTTGGGTGTGGGGACGCCTGCCCCACAAGTGTTTAGGGAGGTCAGTGGGT |
| | TCCTCGCCCGTAGAGACACCGTTTATGCCAAATGAGCACTCCTCATCCCCGCTCTTGA |
| | TGGAGTCATGTCCTAGACGTGAAACTATGGGGCTGTGATCACAAGCAAATGTGTGGGC |
| | GGATCCGTTGCTTGGGTTCTTCCCCGCCCCTCCTTTTTTCGGACCATGACGTCAAGGT |
| | GGGCTGGTGGCGGCAGGTGCGGGGTTGACAATCATACTCCTTTAAGGCGGAGGGATCT |
| | ACAGGAGGGCGGCTGTACTGTGCTTCGCCTTATATAGGGCGACTTGGGGCCCGCAGTA |
| | GCTCTCTCGAGTCACTCCGGCGCAGTGTTGGGACTGTCTGGGTATCGGAAAGCAAGCC |
| | TACGTTGCTCACTATTACGTATAATCCTTTCTTTTCAAGGTAAGGCTGAGATCTCCGC |
| | TAGGCTTCTTTCCCTTTAGTGCTGTATTCGTGTTGTTTTTGTTTTTTTCTGTCCTTTA |
| | GGGAGCCTTAGTCTAGATGTCGGGGTGGCTTGTGGATAACGCTCTGGATTTTTATAGG |
| | GTGAGGGTAGTGGTGGGTGAGGTTTTTTGAGTCCTCCTCGGTTTTCTCTAGTGTGTTT |
| | GGGGGGTGGGGCTTTCTCTCGGCGCCTGCTGGCCGTAGCGAGGTGGGCTGTGGGGTTG |
| | GGGCAGTGGGCGGCTGGCAGCTGCACGTGGTGGCCGCGCGGCCCGGGACGCTGCCATT |
| | TTTGCCCCTCCACTTCCGGACGCGGCTACGGGGCGTCGGAGGGGGACCGCAGGGTGGC |
| | GGGGGTGCCCGCTCGGGTGACTCAGCACGGCCTTGGGGGACTGGCTTTGTCACCTCTC |
| | TTATCGGAATCGATTCTTTGTCCGGATTTAATTGCTCCTCCGGTGGGTATCGTATGGA |
| | TCCCAGGTTATTCCTCCCTGCCCTATGGGGCAGGAGTGTCCCGCCCTTGGACTGGTCT |
| | TAGGAACTGACACCTCAGGGGGAGCAGTTTAAAGTTAGTGCCATTTTTATCTTAAACT |
| | AGTCACTTTGACCTCCCCCAAATAAAGAACTGTAGGTAGTGATTTTCACATTTAAATT |
| | TGTGTAAGGATTACTTGGGATCTCTAGATACCTGGGTTGGACCAACATTATGATTTTT |
| | CTGCCATACTACCAGATGATGCTGAGGCTGCTGGTCACCATTCTTTAAGTAGGTGGGT |
| | TCTGTGACATTTGGTTGAAGAATATTTAGCTTATTTTCTTTTTCCTTCTGAATTTTCA |
| | GGCCTCCCACTTAGTGTGTAGTCTGAGATCTTTAAGAGAATGCATTTTTAGTCTTGGG |
| | AAGGGATAGTACTCCGGTTAAACCAGTCTGAACTCACTGTCTAAGGTCCTAACAAATG |
| | ATATGACCTTTAGGATTTTTAAACATGGGGCCTTAGTGTTCTTTTGTAATTAATGAGA |
| | TTTTTATTTTAGGTCGCCACCATGG |
| 1952 HSP90AB1- | ACGCGTGCTGCCCTGCACTGGTTCCCAGAGACTCCCTCCTTCCCAGGTCCAAATGGCT |
| pGL3 | GCAGGAGCGAAGTGGGCGGAAAAAAAGCGAACCAGCTTGAGAAAGGGCTTGACGTGCC |
| (SEQ ID NO: 5) | TGCGTAGGGAGGGCGCATGTCCCCGTGCTCCGTGTACGTGGCGGCCGCAGGGGCTAGA |
| | GGGGGGTCCCCCCCGCAGGTACTCCACTCTCAGTCTGCAAAAGTGTACGCCCGCAGAG |
| | CCGCCCCAGGTGCCTGGGTGTTGTGTGATTGACGCGGGGAAGGAGGGGTCAGCCGATC |
| | CCTCCCCAACCCTCCATCCCATCCCTGAGGATTGGGCTGGTACCCGCGTCTCTCGGAC |
| | AGGTCAGAGCGGGTCGCCGGGTGGGGTCGCTGCAAAAACCCTGCCCCGGCCGCAGCCG |
| | GAGAGGCGGAGCCTCGCGGGGAGGGGGCGGGACCGCCGAGACAGGCCTGGAAACTGCT |
| | GGAAATGCCGCAGTGCCGCCGCCGCGCCTTCCGCCGCATGTCGGCAAAGAGTCCCCGC |
| | CAGCCCCGGCCGGCGCCCTCCCCCTACGCTGAGCTGCCCCTCAGCGCGAACCCTCCGC |
| | CCTTCCTCTACTCCTGCGAGAGTCGGGATCTGGGGCTACCCAAGGTTGGGTCCCGAAT |
| | GCCAGTCCCTCTGTCGGGACGCGAGATGTGTAGGGCAGATGCTAGGAAGAAGATTGGG |
| | TCTGGGAGCGGTGGTCCGCGTGGTTAGCTGCCTCCGCTCTTTTTCGGTGTCCCCCCCA |
| | GTCCCGCCCTTGGGTGTGGGGACGCCTGCCCCACAAGTGTTTAGGGAGGTCAGTGGGT |
| | TCCTCGCCCGTAGAGACACCGTTTATGCCAAATGAGCACTCCTCATCCCCGCTCTTGA |
| | TGGAGTCATGTCCTAGACGTGAAACTATGGGGCTGTGATCACAAGCAAATGTGTGGGC |
| | GGATCCGTTGCTTGGGTTCTTCCCCGCCCCCTCCTTTTTTCGGACCATGACGTCAAGG |
| | TGGGCTGGTGGCGGCAGGTGCGGGGTTGACAATCATACTCCTTTAAGGCGGAGGGATC |
| | TACAGGAGGGCGGCTGTACTGTGCTTCGCCTTATATAGGGCGACTTGGGGCCCGCAGT |
| | AGCTCTCTCGAGTCACTCCGGCGCAGTGTTGGGACTGTCTGGGTATCGGAAAGCAAGC |
| | CTACGTTGCTCACTATTACGTATAATCCTTTTCTTTTCAAGGTAAGGCTGAGATCTCC |
| | GCTAGGCTTCTTTCCCTTTAGTGCTGTATTCGTGTTGTTTTTGTTTTTTTCTGTCCTT |
| | TAGGGAGCCTTAGTCTAGATGTCGGGGTGGCTTGTGGATAACGCTCTGGATTTTTATA |
| | GGGTGAGGGTAGTGGTGGGTGAGGTTTTTTGAGTCCTCCTCGGTTTTCTCTAGTGTGT |
| | TTGGGGGGTGGGGCTTTCTCTCGGCGCCTGCTGGCCGTAGCGAGGTGGGCTGTGGGGT |
| | TGGGGCAGTGGGCGGCTGGCAGCTGCACGTGGTGGCCGCGCGGCCCGGGACGCTGCCA |
| | TTTTTGCCCCTCCACTTCCGGACGCGGCTACGGGGCGTCGGAGGGGGACCGCAGGGTG |
| | GCGGGGGTGCCCGCTCGGGTGACTCAGCACGGCCTTGGGGGACTGGCTTTGTCACCTC |
| | TCTTATCGGAATCGATGTTAAAGCCTTCTTGGGTGCTTTGTTTCTGTGAGGGAGGGTT |
| | GACGGTGTGGGAAGAGAGCTTTCGGTCTCCAGCACCCGATACTCCCTCCTTCCAGATC |
| | TTTCTTGCAGTCCCGGTGGAGGAGGGGCGGGGAGGGGAGCAGGTTCTGGAAGATTCAT |
| | GGGCTCCTTCCTCCGCCCTTCCTCGAGAGCTGAGATTGTTCTGGAAGCTTCTGGATTC |
| | TGGCGCCCCGCCCCAGTGCCCGGATGCTGGGGCGAGGGAGGGTGCACTGCGGCGCCCC |
| | CTCCTCGCGTGGTCCTGGCCGACGCATGTCCGGCAGTGACGAGTGTCGGCCTGGTGGC |
| | TACGGCCACCATCTTTCTTGGGTTTGGTCCTGTTCTGTAATTTTGTGCTGTGAAGGGG |
| | TCGTGGTGGAGCTTTTGGCTTATCGATTCTTTGTCCGGATTTAATTGCTCCTCCGGTG |
| | GGTATCGTATGGATCCCAGGTTATTCCTCCCTGCCCTATGGGGCAGGAGTGTCCCGCC |
| | CTTGGACTGGTCTTAGGAACTGACACCTCAGGGGGAGCAGTTTAAAGTTAGTGCCATT |
| | TTTATCTTAAACTAGTCACTTTGACCTCCCCCAAATAAAGAACTGTAGGTAGTGATTT |
| | TCACATTTAAATTTGTGTAAGGATTACTTGGGATCTCTAGATACCTGGGTTGGACCAA |
| | CATTATGATTTTTCTGCCATACTACCAGATGATGCTGAGGCTGCTGGTCACCATTCTT |
| | TAAGTAGGTGGGTTCTGTGACATTTGGTTGAAGAATATTTAGCTTATTTTCTTTTTCC |
| | TTCTGAATTTTCAGGCCTCCCACTTAGTGTGTAGTCTGAGATCTTTAAGAGAATGCAT |
| | TTTTAGTCTTGGGAAGGGATAGTACTCCGGTTAAACCAGTCTGAACTCACTGTCTAAG |
| | GTCCTAACAAATGATATGACCTTTAGGATTTTTAAACATGGGGCCTTAGTGTTCTTTT |
| | GTAATTAATGAGATTTTTATTTTAGGTCGCCACCATGG |
| 1953 UBC(v2)- | ACGCGTAGAAGGATGTCGTTCGCTCAGCCTTGCGTTCCAGCTAAAATAAAACTGTGTG |
| pGL3 | GGGTTTCCGCCTCTTTTTTCCAAATTTAACCTGGACACCCAGCTCCTCTGCAGTGTCT |
| (SEQ ID NO: 6) | CCCCTGGAAAGTTCTCGAGCGTTCCCCAGCTTTAGGGCCACGCCCGCCCTGAGATCTG |
| | CCGAGTCATTGTCCTTGTCCCGCGGCCCCGGGAGCCCCCCGCGACCGGCCTGGGAGGC |
| | TCAGGGAGGTTGAAGGGGGCTGAGCAAAGGAAGCCCCGTCATTACCTCAAATGTGACC |
| | CAAAAATAAAGACCCGTCCATCTCGCAGGGTGGGCCAGGGCGGGTCAGGAGGGAGGGG |
| | AGGGAGACCCCGACTCTGCAGAAGGCGCTCGCTGCGTGCCCCACGTCCGCCGAACGCG |
| | GGGTTCGCGACCCGAGGGGACCGCGGGGGCTGAGGGGAGGGGCCGCGGAGCCGCGGCT |
| | AAGGAACGCGGGCCGCCCACCCGCTCCCGGTGCAGCGGCCTCCGCGCCGGGTTTTGGC |
| | GCCTCCCGCGGGCGCCCCCCTCCTCACGGCGAGCGCTGCCACGTCAGACGAAGGGCGC |
| | AGCGAGCGTCCTGATCCTTCCGCCCGGACGCTCAGGACAGCGGCCCGCTGCTCATAAG |
| | ACTCGGCCTTAGAACCCCAGTATCAGCAGAAGGACATTTTAGGACGGGACTTGGGTGA |
| | CTCTAGGGCACTGGTTTTCTTTCCAGAGAGCGGAACAGGCGAGGAAAAGTAGTCCCTT |
| | CTCGGCGATTCTGCGGAGGGATCTCCGTGGGGCGGTGAACGCCGATGATTATATAAGG |
| | ACGCGCCGGGTGTGGCACAGCTAGTTCCGTCGCAGCCGGGATTTGGGTCGCAGTTCTT |
| | GTTTGTGGATCGCTGTGATCGTCACTTGGTGAGTAGCGGGCTGCTGGGCTGGCCGGGG |
| | CTTTCGTGGCCGCCGGGCCGCTCGGTGGGACGGAGGCGTGTGGAGAGACCGCCAAGGG |
| | CTGTAGTCTGGGTCCGCGAGCAAGGTTGCCCTGAACTGGGGGTTGGGGGGAGCGCAGC |
| | AAAATGGCGGCTGTTCCCGAGTCTTGAATGGAAGACGCTTGTGAGGCGGGCTGTGAGG |
| | TCGTTGAAACAAGGTGGGGGGCATGGTGGGCGGCAAGAACCCAAGGTCTTGAGGCCTT |
| | CGCTAATGCGGGAAAGCTCTTATTCGGGTGAGATGGGCTGGGGCACCATCTGGGGACC |
| | CTGACGTGAAGTTTGTCACTGACTGGAGAACTCGGTTTGTCGTCTGTTGCGGGGGCGG |
| | CAGTTATGGCGGTGCCGTTGGGCAGTGCACCCGTACCTTTGGGAGCGCGCGCCCTCGT |
| | CGTGTCGTGACGTCACCCGTTCTGTTGGCTTATAATGCAGGGTGGGGCCACCTGCCGG |
| | TAGGTGTGCGGTAGGCTTTTCTCCGTCGCAGGACGCAGGGTTCGGGCCTAGGGTAGGC |
| | TCTCCTGAATCGACAGGCGCCGGACCTCTGGTGAGGGGAGGGATAAGTGAGGCGTCAG |
| | TTTCTCTGGTCGGTTTTATGTACCTATCTTCTTAAGTAGCTGAAGCTCCGGTTTTGAA |
| | CTATGCGCTCGGGGTTGGCGAGTGTGTTTTGTGAAGTTTTTTAGGCACCTTTTGAAAT |
| | GTAATCATTTGGGTCAATATGTAATTTTCAGTGTTAGACTAGTAAATTGTCCGCTAAA |
| | TTCTGGCCGTTTTTGGCTTTTTTGTTAGGTGCCACCATGG |
| 1954 EEFlal-pGL3 | ACGCGTAGCTTCGTGAGGCTCCGGTGCCCGTCAGTGGGCAGAGCGCACATCGCCCACA |
| (SEQ ID NO: 7) | GTCCCCGAGAAGTTGGGGGGAGGGGTCGGCAATTGAACCGGTGCCTAGAGAAGGTGGC |
| | GCGGGGTAAACTGGGAAAGTGATGTCGTGTACTGGCTCCGCCTTTTTCCCGAGGGTGG |
| | GGGAGAACCGTATATAAGTGCAGTAGTCGCCGTGAACGTTCTTTTTCGCAACGGGTTT |
| | GCCGCCAGAACACAGGTAAGTGCCGTGTGTGGTTCCCGCGGGCCTGGCCTCTTTACGG |
| | GTTATGGCCCTTGCGTGCCTTGAATTACTTCCACCTGGCTCCAGTACGTGATTCTTGA |
| | TCCCGAGCTGGAGCCAGGGGCGGGCCTTGCGCTTTAGGAGCCCCTTCGCCTCGTGCTT |
| | GAGTTGAGGCCTGGCCTGGGCGCTGGGGCCGCCGCGTGCGAATCTGGTGGCACCTTCG |
| | CGCCTGTCTCGCTGCTTTCGATAAGTCTCTAGCCATTTAAAATTTTTGATGACCTGCT |
| | GCGACGCTTTTTTTCTGGCAAGATAGTCTTGTAAATGCGGGCCAGGATCTGCACACTG |
| | GTATTTCGGTTTTTGGGCCCGCGGCCGGCGACGGGGCCCGTGCGTCCCAGCGCACATG |
| | TTCGGCGAGGCGGGGCCTGCGAGCGCGGCCACCGAGAATCGGACGGGGGTAGTCTCAA |
| | GCTGGCCGGCCTGCTCTGGTGCCTGGCCTCGCGCCGCCGTGTATCGCCCCGCCCTGGG |
| | CGGCAAGGCTGGCCCGGTCGGCACCAGTTGCGTGAGCGGAAAGATGGCCGCTTCCCGG |
| | CCCTGCTCCAGGGGGCTCAAAATGGAGGACGCGGCGCTCGGGAGAGCGGGCGGGTGAG |
| | TCACCCACACAAAGGAAAAGGGCCTTTCCGTCCTCAGCCGTCGCTTCATGTGACTCCA |
| | CGGAGTACCGGGCGCCGTCCAGGCACCTCGATTAGTTCTGGAGCTTTTGGAGTACGTC |
| | GTCTTTAGGTTGGGGGGAGGGGTTTTATGCGATGGAGTTTCCCCACACTGAGTGGGTG |
| | GAGACTGAAGTTAGGCCAGCTTGGCACTTGATGTAATTCTCGTTGGAATTTGCCCTTT |
| | TTGAGTTTGGATCTTGGTTCATTCTCAAGCCTCAGACAGTGGTTCAAAGTTTTTTTCT |
| | TCCATTTCAGGTGTCGTGAACACGTGGTCGCGGCCAAGATCTAGTACTAGTGGGCCAC |
| | CATGG |
| UBB-pGL3 | ACGCGTGCTAGCCCGGGCTCGAGATCTAGACCCCTCCTCCTTCTCCCGCCGGAAATAC |
| (SEQ ID NO: 8) | CCTCTTTCAGGACGGCGCGCCTGTGCGGCGCACGCGCGCTCAGTTACTTAGCAACCTC |
| | GGCGCTAAGCCACCCCAGGTGGAGCCCAGCAACAACAGAGCCACCGCGTCCCCCACCA |
| | ATCAGCGCCGACCTCGCCTTCGCAGGCCTAACCAATCAGTGCCGGCGCTGCAAGGAAG |
| | TTTCCAGAGCTTTCGAGGAAGGTTTCTTCAACTCAAATTCATCCGCCTGATAATTTTC |
| | TTATATTTTCCTAAAGAAGGAAGAGAAGCGCATAGAGGAGAAGGGAAATAATTTTTTA |
| | GGAGCCTTTCTTACGGCTATGAGGAATTTGGGGCTCAGTTGAAAAGCCTAAACTGCCT |
| | CTCGGGAGGTTGGGCGCGGCGAACTACTTTCAGCGGCGCACGGAGACGGCGTCTACGT |
| | GAGGGGTGATAAGTGACGCAACACTCGTTGCATAAATTTGCGCTCCGCCAGCCCGGAG |
| | CATTTAGGGGCGGTTGGCTTTGTTGGGTGAGCTTGTTTGTGTCCCTGTGGGTGGACGT |
| | GGTTGGTGATTGGCAGGATCCTGGTATCCGCTAACAGGTACTGGCCCGCAGCCGTAAC |
| | GACCTTGGTGGGGTGTGAGAGGGGGGAATGGGTGAGGTCAAGGTGGAGGCTTCTTGGG |
| | GTTGGGTGGGCCGCTGAGGGGAGGGCGTGGGGGAAGGGAGGGCGAGGTGACGCGGCGC |
| | TTGGCTTTTCCGGAACAGTGGGCCTTGTTGACTTGAGGAGGGCGAGTGCGGTTGGCGC |
| | GCGCGCGCGTTGACGGAAACTAACGGACGTCTAACCGATCGGCGATTCTGTCGAGTTT |
| | ACTTCGCGGGGAAGGCGGAAAAGAGGTAGTTTGTGTGGTTTCTGGAAGCCTTTACTTT |
| | GGAATCCCAGTGTGAGAAAGGTGCCCCTTCTTGTGTTTCAATGGGATTTTTATTTCGC |
| | GAGTCTTGTGGGTTTGGTTTTGTTTTCAGTTTGCCTAACACCGTGCTTAGGTTTGAGG |
| | CAGATTGGAGTTCGGTCGGGGGAGTTTGAATATCCGGAACAGTTAGTGGGGAAAGCTG |
| | TGGACGCTTGGTAAGAGAGCGCTCTGGATTTTCCGCTGTTGACGTTGAAACCTTGAAT |
| | GACGAATTTCGTATTAAGTGACTTAGCCTTGTAAAATTGAGGGGAGGCTTGCGGAATA |
| | TTAACGTATTTAAGGCATTTTGAAGGAATAGTTGCTAATTTTGAAGAATATTAGGTGT |
| | AAAAGCAAGAAATACAATGATCCTGAGGTGACACGCTTATGTTTTACTTTTAAACTAG |
| | GTCACCATGG |
| RPSA-pGL3 | ACGCGTGCTAGCCCGGGCTCGAGATCTAAAAGATAGATATAATCCATACCATCTGTTC |
| (SEQ ID NO: 9) | AATCTTGTCCTTTTCAACTTTTTCAGGGAAACTTCCCCCAGGTGATAGATGGATAGAT |
| | ACATGAGATAGACATAACGCATACGATCAGTTCAATCTTATCTTTTTTAACTTTTTCA |
| | GAGAAAACTTCCCTTAAGTGAACATTTAAATCTGAATTACGTCCTGTTAAACTGTTCT |
| | CCAGGAAAATGAAATAAAATAAATCTTCAAGTTTTTGTTTACCTAACAATTTGTTGTG |
| | TCGAACAAACCTTCCTACTTTTCAGGTAACAAAATGGCAGCTTAGGCTAGAAAGCCGC |
| | TCATATTCGCAGGTACAAGGGCTGGGTAAGAACGCCCCGCCTGGCTGACTAACTTGAG |
| | TTCCGCGCTCTGGACAGGAATTATGCACAGGGCGTCGCTGTGGCACTAGAAACCCCAA |
| | AGTCACAAGCGCCCCAGATCCGACCAGGATGCCGCTACCGGCTACAGCCCAGAGGCCC |
| | GCTCCTGCGGCGCAAGCCCGCCTTCCTGAAGAAAAAGCCCAGTCCCGGCAGCGTTCTT |
| | CTCCGGCTCCGCCCTTCTTCCGCTCGACTTTCTTTGCCATTGGCTGACAACGGAGTAC |
| | ATAAGGACGTCATTTCCTGCCGCCTGTCTTTTCCGTGCTACCTGCAGAGGGGTCCATA |
| | CGGCGTTGTTCTGGGTGAGTTCCGTGTAGCGTCCCTGGCGCCTTCCAGGGCTAGAAAA |
| | ATGAGCTTTTCCTGCTCAAATGAAGGGTGAGAAGACTAGTGATGAAAGCCGGTCAGAC |
| | TGGATCTGTCTTCCGCCCGGCGCGCCCCACTTTAGGCCTGCGGCCCGCACATGGCCAG |
| | GCTCGGGCTGGCGGGTTCCCAGAGTGCTCCGGGAGCGGGTGGAGGTCGCCCTCCAGCG |
| | GAGGCTCCGAGCTGGGGTTCGGACCAGGCCGCGGGTGGGCGGGAGTGCAGAAAGCGGG |
| | CTAACATCCTGTGTTGCTATCCCTTCGGAGTCCCACACGGCGGTGAGTCTAGGCCCAG |
| | GCGCTGATTTACACCAGCTACTTGGGCTGGTCGGGTTTTCCCTTCGCGCCGTGCGGGT |
| | CAGGAGTTAAGGTTCTCGGGTTTTTAGACAAACAAGTGGTGACAGCACAGCGAAGTAA |
| | TTCCAAAGCATCCGCCTACAATCTGCTTGAAAATGTCTGAAAACAATTCATGCCTTTT |
| | TTGCCTTTAGTTTGCATATTCCAAACATGGCTGCTCTTTTGTATCTAGTTGTTAACTT |
| | GGCGCATCCACAACTTTTCCTTAATTCCTATCTTGAGAAGTGTTGAATTTCCATTCGC |
| | TAATTTCGTGTAGTTTTATTACTCGGTTACTCTGCCGTCCACACTATTTCCTCAGTAA |
| | GATGTGCGCTGTTCCGTAATACACGACATGTATGGGTTAACTTTCTGTTTACCCTTCA |
| | CTACACTGTAAGCTCAATGCCTGACACTATAGCAGATGGAGTTTTTGGTTGCTTTTAA |
| | GGGTGTGCCCTACTTAACTCAATGGAATGAAAAGAAATAGGTTGCTCTCTTATTTCAG |
| | ATTCCCGTCGTAACTTAAAGGGAAATTTTCACCATGG |
| NACA-pGL3 | ACGCGTGCTAGCCCGGGCTCGAGATCTTCATTTATTTAGGGAGACAGAGGAGTTTTTA |
| (SEQ ID NO: 10) | GCTGGATTACGTTATCCATACAAGGTTGGAACATAATTACAGAATTTGGTTACAAGAA |
| | GTGTTTTTTTTGGTGGGTGGGCGGGGGTACAGCTAACATTGTTTTACGAAGAGTTTAA |
| | CACGCATGAGTTGCTGTCATCTGGTGACATCGTTTGAGTCTCTCTAGTCATTGAACAG |
| | AACAAGAAAAATCGAATTAAATTTATAATCTGAACTGAAGTTATATGTGACCCATCAC |
| | ATCTCTCAAGTTTTAAAATGGGTTTTTTTGTTGTTGTTGATGGAGGGGGAGAGGGTCC |
| | AGCAGUTTTTAAATGTTTTCACATCGTGTGTTCCAAAAATAACTGGTTAGCCTAAGTC |
| | ACTTCCACCCTCCAATGTTGTGAATGCAGTCTCTAGCATTCGCTATTTAATGTCTTCT |
| | TCCTGCACTATTTGAGAAATCGCGAGGTCGACTTAATACCGCAGTCGCCACTTCGCGG |
| | ACCGGAGGGCGGAGTCTGCTTAGTTCTGAGGACTGCGTGGGTCCGCGCAGAGAGCTCC |
| | TGCTAGGCCTGCGCGTCCCGTTCTAAATTCTTACCCTTTAGTCCTTGTCACCACCCCC |
| | GCCGTGGGAACGGCCTGACAGTCACTCGTCAAAGGAAGTGGCTGCCGGCAGCTCTTGA |
| | CCCGGAATCGGATCCTAGTCCCACCCCCTCCGCTCCAGGCTTCCTTCTGCAACAGGCG |
| | TGGGTCACGCTCTCGCTCGGTCTTTCTGCCGCCATCTTGGTTCCGCGTTCCCTGCACA |
| | GTAAGTACTTTCTGTGCCGCTACTGTCTATCCGCAGCCATCCGCCTTTCTTTCGGGCT |
| | AAGCCGCCCCGGGGACTGAGAGTTAAGGAGAGTTGGAGGCTTTACTGGGCCACAGGGT |
| | TCCTACTCGCCCCTGGGCCTCCGGACAAAATGGGGTCTGCGGTTGGTGTCCTGGCAAA |
| | AGCAGGGTAGAAGGGCTGCGGGGCGGGCCCAGAATCCGAGCCTGCAGAGATGGGAGCA |
| | GTTGCAGTGTTGAGGGCGGAAGAGGAGTGCGTCTTGTTTTGGGAACTGCTTCACAGGA |
| | TCCAGAAAAGGGTAAGGGGTCACAGCCTTAGAACCTGTAACACCGTTCTCCCTGTGAC |
| | AAGCAAGTGTTGGACTTAAAACACCGTCTTTCCCCTCCTGGTACCCCAAAGTCGGCAA |
| | ACGTAGTCCAGGAGGCCCCAGCCCAGCCAGTGTGAGTATTAGGAGTGGAGGGGGTTCA |
| | CAGTAGCGTCTGAAGTCTCCCATGATCGAGAGCCAGCCCGGGATCCTCTCCCTCGGGT |
| | TGAGAATGCAGTGGGAATTGCTGGCCTTGATAGAGGCGTGAGGGTCACATCATATTTA |
| | CCTCTTATTCCCAGGTGCTTTGGGGAAGGTTGTCACCAAACACCCTCACGATTTTTTT |
| | CTAACAGCCCTCCTCGGAGTTTTTAAGAATACTTATCTCCGGGTTGGGCAAGTGAATG |
| | TATCCCAATCAATTTAGGCCTCCTTTTTATTCCCTTCCTTCAGCCATGG |
| COX8A | ACGCGTAGCCTGAATAGAGAGAGATTTAACAGTATGAATGAAGGGAGGAGTCAAGAAC |
| (SEQ ID NO: 11) | AGTTTAGGCTTCTCGCGTACATGATTGGGTGGCCCCAGATGTCATTCACAAGTATAAG |
| | CCTTGGAGGCGGAACATAACTCGAAGAAGAGCCCTTTTGTGCTCCGCCCATAGCGTAG |
| | GAAGGTGTCAATTGGCTGTTTCGAGGAACGCGCCAAAAACTGCCAAGGGCTGTGGGAG |
| | GTGTGTTCTTGCGTCATTTCCGAGAGACTTCCGCGCCGCAGTTTCCCTGCTTCCCCAG |
| | CTCCAGAACTTCCGGCCAGCGCAGCCATTTTGGCTTCCTGACCTTGGGCTACGGCTGA |
| | CCGTTTTTTGTGGTGTACTCCGTGCCATCAAATCCGTCCTGACGCCGCTGCTGCTGCG |
| | GGGCTTGACAGGCTCGGCCCGGCGGCTCCCAGTGCCGCGCGCCAAGATCCATTCGTTG |
| | CCGCCGGAGGGGAAGCTTGCCACCATGG |
| ACTB | ACGCGTCGTTGGCAGGTCCTGAGGCAGCTGGCAAGACGCCTGCAGCTGAAAGATACAA |
| (SEQ ID NO: 12) | GGCCAGGGACAGGACAGTCCCATCCCCAGGAGGCAGGGAGTATACAGGCTGGGGAAGT |
| | TTGCCCTTGCGTGGGGTGGTGATGGAGGAGGCTCAGCAAGTCTTCTGGACTGTGAACC |
| | TGTGTCTGCCACTGTGTGCTGGGTGGTGGTCATCTTTCCCACCAGGCTGTGGCCTCTG |
| | CAACCTTCAAGGGAGGAGCAGGTCCCATTGGCTGAGCACAGCCTTGTACCGTGAACTG |
| | GAACAAGCAGCCTCCTTCCTGGCCACAGGTTCCATGTCCTTATATGGACTCATCTTTG |
| | CCTATTGCGACACACACTCAGTGAACACCTACTACGCGCTGCAAAGAGCCCCGCAGGC |
| | CTGAGGTGCCCCCACCTCACCACTCTTCCTATTTTTGTGTAAAAATCCAGCTTCTTGT |
| | CACCACCTCCAAGGAGGGGGAGGAGGAGGAAGGCAGGTTCCTCTAGGCTGAGCCGAAT |
| | GCCCCTCTGTGGTCCCACGCCACTGATCGCTGCATGCCCACCACCTGGGTACACACAG |
| | TCTGTGATTCCCGGAGCAGAACGGACCCTGCCCACCCGGTCTTGTGTGCTACTCAGTG |
| | GACAGACCCAAGGCAAGAAAGGGTGACAAGGACAGGGTCTTCCCAGGCTGGCTTTGAG |
| | TTCCTAGCACCGCCCCGCCCCCAATCCTCTGTGGCACATGGAGTCTTGGTCCCCAGAG |
| | TCCCCCAGCGGCCTCCAGATGGTCTGGGAGGGCAGTTCAGCTGTGGCTGCGCATAGCA |
| | GACATACAACGGACGGTGGGCCCAGACCCAGGCTGTGTAGACCCAGCCCCCCCGCCCC |
| | GCAGTGCCTAGGTCACCCACTAACGCCCCAGGCCTTGTCTTGGCTGGGCGTGACTGTT |
| | ACCCTCAAAAGCAGGCAGCTCCAGGGTAAAAGGTGCCCTGCCCTGTAGAGCCCACCTT |
| | CCTTCCCAGGGCTGCGGCTGGGTAGGTTTGTAGCCTTCATCACGGGCCACCTCCAGCC |
| | ACTGGACCGCTGGCCCCTGCCCTGTCCTGGGGAGTGTGGTCCTGCGACTTCTAAGTGG |
| | CCGCAAGCCACCTGACTCCCCCAACACCACACTCTACCTCTCAAGCCCAGGTCTCTCC |
| | CTAGTGACCCACCCAGCACATTTAGCTAGCTGAGCCCCACAGCCAGAGGTCCTCAGGC |
| | CCTGCTTTCAGGGCAGTTGCTCTGAAGTCGGCAAGGGGGAGTGACTGCCTGGCCACTC |
| | CATGCCCTCCAAGAGCTCCTTCTGCAGGAGCGTACAGAACCCAGGGCCCTGGCACCCG |
| | TGCAGACCCTGGCCCACCCCACCTGGGCGCTCAGTGCCCAAGAGATGTCCACACCTAG |
| | GATGTCCCGCGGTGGGTGGGGGGCCCGAGAGACGGGCAGGCCGGGGGCAGGCCTGGCC |
| | ATGCGGGGCCGAACCGGGCACTGCCCAGCGTGGGGCGCGGGGGCCACGGCGCGCGCCC |
| | CCAGCCCCCGGGCCCAGCACCCCAAGGCGGCCAACGCCAAAACTCTCCCTCCTCCTCT |
| | TCCTCAATCTCGCTCTCGCTCTTTTTTTTTTTCGCAAAAGGAGGGGAGAGGGGGTAAA |
| | AAAATGCTGCACTGTGCGGCGAAGCCGGTGAGTGAGCGGCGCGGGGCCAATCAGCGTG |
| | CGCCGTTCCGAAAGTTGCCTTTTATGGCTCGAGCGGCCGCGGCGGCGCCCTATAAAAC |
| | CCAGCGGCGCGACGCGCCACCACCGCCGAGACCGCGTCCGCCCCGCGAGCACAGAGCC |
| | TCGCCTTTGCCGATCCGCCGCCCGTCCACACCCGCCGCCAGGTAAGCCCGGCCAGCCG |
| | ACCGGGGCAGGCGGCTCACGGCCCGGCCGCAGGCGGCCGCGGCCCCTTCGCCCGTGCA |
| | GAGCCGCCGTCTGGGCCGCAGCGGGGGGCGCATGGGGGGGGAACCGGACCGCCGTGGG |
| | GGGCGCGGGAGAAGCCCCTGGGCCTCCGGAGATGGGGGACACCCCACGCCAGTTCGGA |
| | GGCGCGAGGCCGCGCTCGGGAGGCGCGCTCCGGGGGTGCCGCTCTCGGGGCGGGGGCA |
| | ACCGGCGGGGTCTTTGTCTGAGCCGGGCTCTTGCCAATGGGGATCGCAGGGTGGGCGC |
| | GGCGGAGCCCCCGCCAGGCCCGGTGGGGGCTGGGGCGCCATTGCGCGTGCGCGCTGGT |
| | CCTTTGGGCGCTAACTGCGTGCGCGCTGGGAATTGGCGCTAATTGCGCGTGCGCGCTG |
| | GGACTCAAGGCGCTAACTGCGCGTGCGTTCTGGGGCCCGGGGTGCCGCGGCCTGGGCT |
| | GGGGCGAAGGCGGGCTCGGCCGGAAGGGGTGGGGTCGCCGCGGCTCCCGGGCGCTTGC |
| | GCGCACTTCCTGCCCGAGCCGCTGGCCGCCCGAGGGTGTGGCCGCTGCGTGCGCGCGC |
| | GCCGACCCGGCGCTGTTTGAACCGGGCGGAGGCGGGGCTGGCGCCCGGTTGGGAGGGG |
| | GTTGGGGCCTGGCTTCCTGCCGCGCGCCGCGGGGACGCCTCCGACCAGTGTTTGCCTT |
| | TTATGGTAATAACGCGGCCGGCCCGGCTTCCTTTGTCCCCAATCTGGGCGCGCGCCGG |
| | CGCCCCCTGGCGGCCTAAGGACTCGGCGCGCCGGAAGTGGCCAGGGCGGGGGCGACCT |
| | CGGCTCACAGCGCGCCCGGCTATTCTCGCAGCTCACAGATCTAGTACTAGTGGGCCAC |
| | CATGG |
| UBA52v2 | AAATCCTGAGTCCCGCTTGACACCTTTTGTCAGGCACCACCACCTTTCTGGGCGAATG |
| (SEQ ID NO: 17) | CGGTAGTACCGTCTGCTCTCCCTGCTGCTGTCCTGAAATCCATTCAGGCACAGCGGCC |
| | GAGAGCTTTATAATAACCGATTCCAGGTGTTAGGTGCTTTCCCAGCCCCGACTCCTGC |
| | GTCCTGGACCCGCAGTCCTCTGCTTAATACCTTTGCTTTATTAGAAAACATTCTCCTC |
| | TACTCCGTTCAGCTATTCGCTGAGGGCCCGCCAACCGCCAGCGGTTGTCAGTGGCCTA |
| | GAGGCAGCGGACGCAAACACGGGGAGAGGTGCAATCGTCTCAAGTGACTCGGCGGGCG |
| | GGGCCCACAACCGGAAGCGGGTGGGCGACCTTCACCCACGTGCGCTGCGGCTTCGTTC |
| | GCCAGCATCCAAGATGGCGGCAGGGCGGGGCCCAAGGCGCGGCGCGAATTGTGACGCA |
| | GGCGTCCGGCGTGCTCCGTGCGCAAGCGCTTTCGGCGGCGATTAGGTGGTTTCCGGTT |
| | CCGCTATCTTCTTTTTCTTCAGCGAGGCGGCCGAGCTGGTTGGTGGCGGCGGTCGTGC |
| | GGGTTCGCGCCGGGCCGAGAGCGGGTTGGGGGCTGCGGGAGGCTGCAGGGGCCTGGGC |
| | GGCAGAAGAGGCGGCCCTGAGCTGGCTCATGCGGGCCAGTCTCGGCAGGGTGGCTGGG |
| | CAGGGCTCGCGAGGCCACGGCTCGGAGCCCAGACCGGGGCCCAGGAGGCGAGCGCCGT |
| | TTTGGAGAGGAGCCTGCCTGCTCTGCCTGCCAGCGTGACCCCACGAGGCCTCGGGCGG |
| | GAAGAGGTCCTCGGGGCAGATCCGAGTTAATGAGAGAGGGGTATTGAGCGTGTAGCGT |
| | TAACTCTGCCAGTCACTGCGTCAGTCGCTTTGGAAATACTAAATTTCTCGAGCTGAGT |
| | CTTCATACCTGGCTCCATTACTACGTCTGTAAGGAGGAGCTGGTGGTAGTGTCTGCTT |
| | TTTAGACTTTTCTTTAGACTATTTGTATTTTTTTCAGATGGAGTCTTGCTCTGTCGCC |
| | TAAGCTGGAGTTCAGTGGTGCGGTCTCGGCTCACTGCAATCTCCACCTCCCGGGCTCG |
| | AGCGATTCTTCTGCCTCAGCCTCCCGAGTAGCTGGGATTATAGGCGCCTGCCACCACG |
| | CCCAGTTGATTTTTGTAGTTTTAGTAGAGACGGAGTTTCACCATGTTAGCCAGGCTCA |
| | TCTTGAACTCTTGACCTCAAATGATCCGTCTGCCTCGGCCTTCCAAAGTGCTGGGATT |
| | ACAGGCATGAGCCCCTGCGCCCGGTCGATTCTTTGTCTTTTTAAGTCAACTTTTATAT |
| | GTGAACAATGCTTGGCAGGTGGTTGGTAGATACTAAGTGATGTTCGTGGTTTGGGGTC |
| | AAGGCAAGAAGTGGGGTCTGGAGAGTTTTGGTGTAATTGAGAAGGAAGCTAAGAGTGT |
| | TGGGTGCTCCAGCTTGGAGTTAGAGAGGAGAGAGGCTGCGACAGGAAGGCATGTGTGT |
| | TGTAGGGGATGGCTTCCCATCCAGGCTGGCAGCAGGAGCAGCCTGTGCAGATCAGGAC |
| | CTGGCTGCCGTGGAAGAGGGTGGGACCGCCTTCAGGGAAGATGGATCTAGCAAGTTGA |
| | AGCCAAAGGGTACTTATTCCATCAGGAGATACTGACGAGTCCTTCCGCCGCTAAACCT |
| | AAGGAGAATAACCACAGTCTGTGTTCCTGAAGAGCACCCGTGCGGTCAGGAGGGTGGA |
| | GGACATGTGGTCTTTAGTTCCAGGACATGTTTAGACTACAGGCCAGGGTGTGTGAGAA |
| | GCCTAGCAGGGCCAGGCTTGGAGGAGTGAAAGGAAGACAGGTACTGGGGCAGGACCAG |
| | TTGGACTTGGTGCAGGCAAAGGGATAGCAACTGTGGTGTAGGCACCTGAGCTTGTGCT |
| | ACTCAGGCATGCATTGCTCACCAGTCTATCCTGCCTCCCTTCCTCCTGCAGACGCAAC |
| | CATGG |

Luciferase expression during transient transfection: Transient transfection was performed of the promoter-pGL3 plasmids into iPSCs to determine the strength of expression. Using a 96wp format, 50 uL of E8 media+10 uM blebbistatin was added to each well. Each plasmid was assayed in triplicate by adding 16.5 uL of the following reagent preparation. One well of a 6wp of iPSC line 01279.107 was harvested using Accutase, resuspended in 3.5 mL E8 media+10 uM blebbistatin, and 50 uL was added to each well. One day later the cells were assayed using the Dual-Luciferase Reporter Assay System (Promega).

TABLE 13
Reagent composition.

| Reagent | Description | μL for 4 rxns |
|---|---|---|
| DNA 1 | 10 ng pGL4.75 | 40 ng (4.0 uL) |
| | (hRluc/CMV) | |
| DNA 2 | 140 ng promoter plasmid | 560 ng (~1 uL) |
| Lipofection Reagent | TransIT-LTI | 1.8 uL |
| Basal Medium or OptiMEM | OptiMEM | 60 uL |

Normalized luciferase (Firefly/Renilla ratio, normalized to EEF1A1=100%) are displayed below (HSP90AB 1de1400 promoter and HSP90AB1 promoter had expression around 66% and 75% of EEF1A1). Expression values at or above the level of the PGK promoter were desired so RPS19, UBA52, HSP90AB1, and UBC were selected for further study.

ZsGreen Constructs Integrated at the AAVS1 Safe Harbor Locus: To examine longer term expression driven by the candidate promoters in a chromosomal context, they were cloned into plasmids controlling ZsGreen fluorescent protein and targeted to the AAVS1 safe harbor locus on chromosome 19 in human iPSC (plasmid design is shown below using the CAG promoter as an example). The plasmids were integrated into iPSC line 01279.107 via CRISPR-mediated gene editing, puromycin selection was applied, and resistant colonies were picked and genotyped by PCR. Correctly targeted heterozygous clones were expanded.

TABLE 14
Clones generated.

| iPSC Clone | Promoter | Targeting Plasmid |
|---|---|---|
| 5353 | CAG | 1286 pZD-SA-PuroR/CAGp-ZsGreen |
| 5355 | UBC(v1) | 1288 pZD-SA-PuroR/UBCp(v1)- ZsGreen |
| 5358, 5359 | UBC(v2) | 1962 pZD-SA-PuroR/UBC(v2)- ZsGreen |
| 5361, 5362 | UBA52 | 1963 pZD-SA-PuroR/UBA52- ZsGreen |
| 5363, 5364 | RPS19 | 1964 pZD-SA-PuroR/RPS19- ZsGreen |
| 5365, 5366 | HSP90AB1(del400) | 1965 pZD-SA-PuroR/HSP90AB1a- ZsGreen |

Genomic Loci Suitable for Tagging to Yield Constitutive Expression: In addition to promoter-driven expression from a safe harbor, specific genes expressed in most cell-types can be tagged with a reporter gene to give constitutive expression. The genes HSP90AB1, ACTB, CTNNB1, and MYL6 were chosen for evaluation and were tagged with ZsGreen and an F2A cleavage sequence via TALEN-mediated gene editing. Correctly targeted heterozygous clones were expanded.

TABLE 15
iPSC clones and gene loci.

| iPSC Clone | Gene Locus | Targeting Plasmid |
|---|---|---|
| 5388, 5427 | HSP90AB1 | 1978 pTD-HSP90AB1-F2A-ZsGreen |
| 5431, 5432 | ACTB | 1979 pTD-ZsGreen-F2A-ACTB |
| 5433, 1980-1 | CTNNB1 | 1980 pTD-ZsGreen-F2A-CTNNB1 |
| 1981-3, 1981-6 | MYL6 | 1981 pTD-ZsGreen-F2A-MYL6 |

ZsGreen expression in iPSC: Engineered iPSC lines expressing ZsGreen fluorescent protein were maintained in culture for up to seven months (E8 media/vitronectin coated plates) and periodically checked for green expression using flow cytometry on an Accuri C6 instrument (BD). Most clones maintained a consistent flow profile over time, apart from one of the RPS19 promoter clones (5363), which showed many cells with lowered fluorescence at the August time point.

Differentiation: In order to determine the stability of expression post-differentiation, the engineered lines were subjected to differentiation protocols to direct them toward either neuronal or cardiac cell types.

TABLE 16
Neuronal protocol.

| Day | Action |
|---|---|
| −2 | EDTA split iPSC lines |
| −1 | |
| 0 | Changed media with DMEMF12:Neurobasal (1:1) + B27 − VitA + Glutamax + |
| | LDN193189 (200 nM) + SB431542 (10 uM) |
| 1 | Changed media with DMEMF12:Neurobasal 1:1) + B27 − VitA + Glutamax + |
| | LDN193189 (200 nM) + SB431542 (10 uM) |
| 2 | Changed media with DMEMF12:Neurobasal (1:1) + B27 − VitA + Glutamax + |
| | LDN193189 (200 nM) + SB431542 (10 uM) |
| 3 | Changed media with DMEMF12:Neurobasal (1:1) + B27 − VitA + Glutamax + |
| | LDN193189 (200 nM) + SB431542 (10 uM) |
| 4 | Changed media with DMEMF12:Neurobasal (1:1) + B27 − VitA + Glutamax + |
| | LDN193189 (200 nM) + SB431542 (10 uM) |
| 5 | Changed media with DMEMF12:Neurobasal (1:1) + B27 − VitA + Glutamax + |
| | LDN193189 (200 nM) + SB431542 (10 uM) |
| 6 | Changed media with DMEMF12:Neurobasal (1:1) + B27 − VitA + Glutamax + |
| | LDN193189 (200 nM) + SB431542 (10 uM) |
| 7 | Dissociated with TrypLE and plated each sample across 2 wells of a 6wp ultra |
| | low attachment plate in 3 mL per well: |
| | DMEMF12:Neurobasal (1:1) + B27 − VitA + Glutamax + H1152 (1 uM) |
| 9 | Settled aggregates, removed most media, added DMEMF12:Neurobasal (1:1) + |
| | B27 − VitA + Glutamax |
| 11 | Settled aggregates, removed most media, added DMEMF12:Neurobasal (1:1) + |
| | B27 − VitA + Glutamax |
| 13 | Settled aggregates, removed most media, added DMEMF12:Neurobasal (1:1) + |
| | B27 − VitA + Glutamax |
| 14 | Dissociated the remaining aggregates and plated on PLO-Laminin 12wp with |
| | DMEMF12:Neurobasal (1:1) + B27 − VitA + |
| | Glutamax + DAPT (5 uM) + H1152 (1 uM) |
| 16 | Media change 2 mL per well: |
| | DMEMF12:Neurobasal (1:1) + B27 − VitA + Glutamax + DAPT (5 uM) |
| 18 | Media change 2 mL per well: |
| | DMEMF12:Neurobasal (1:1) + B27 − VitA + Glutamax + DAPT (5 uM) |
| 20 | Media change 2 mL per well: |
| | DMEMF12:Neurobasal (1:1) + B27 − VitA + Glutamax + DAPT (5 uM) |
| 21 | Harvested 1 well with accutase (30 min at 37C). Performed flow cytometry |

At day 21 of differentiation, all cells had a visible neuronal phenotype. Flow cytometry showed many cells with diminishing fluorescence for the CAG, UBC(v1), and HSP90AB1de1400 promoters. The UBCv2, UBA52, and RPS19 promoters showed tight and stable expression, as did the tagged genes HSP90AB 1, CTNNB1, and MYL6.

TABLE 17
Cardiac protocol.

| Day | Action |
|---|---|
| −1 | EDTA split line into duplicate 24wp in E8/VTN (1:12 from 6wp = 1:3 onto 24wp) |
| 0 | Change media: 1 ml per well RPMI/B27 minus insulin/glutamax/CHIR99021 (7 |
| | uM) |
| 1 | Change media @ 24 h with 1 ml per well RPMI/B27 minus insulin/glutamax |
| 2 | Change media with RPMI/B27 minus insulin/IWP2 (5 uM )/glutamax |
| 3 | No action |
| 4 | Changed media with RPMI/B27 minus insulin/glutamax |
| 5 | No action |
| 6 | Changed media with RPMI/B27 minus insulin/glutamax |
| 8 | Change media with RPMI/B27/glutamax |
| 11 | Change media with RPMI/B27/glutamax |
| 13 | Change media with RPMI/B27/glutamax |
| 16 | Change media with RPMI/B27/glutamax |
| 19 | Change media with RPMI/B27/glutamax |
| 21 | Performed flow on one plate. Excluded dead cells by staining with Live/Dead red. |

At day 21 of differentiation the CAG, UBC(v1), RPS19, and HSP90AB1de1400 promoter lines showed varying amounts of expression silencing. The UBC(v2) and UBA52 promoters showed tight and stable expression, as did the tagged genes HSP90AB1, ACTB, CTNNB1, and MYL6.

The newly generated promoter regions UBCv2, UBA52, RPS19, and HSP90AB1de1400 gave stable iPSC expression over four months in culture, and out to seven months with only RPS19 showing some silencing at this point. The gene loci HSP90AB 1, ACTB, CTNNB1, and MYL6 were shown to give stable expression of the tagged ZsGreen reporter. The UBCv2 and UBA52 reporters were shown to be stable under two different differentiation protocols, as well expression driven from the genes HSP90AB1, CTNNB1, and MYL6.

All methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Alexander et al., Proc. Nat. Acad. Sci. USA, 85:5092-5096,1988.
Ausubel et al., Current Protocols in Molecular Biology, Greene Publ. Assoc. Inc. & John Wiley & Sons, Inc., Mass., 1996.Blomer et al., 1997
Chen and Okayama, Mol. Cell Biol., 7(8):2745-2752, 1987.
Chen et al., Nature Methods 8:424-429, 2011.
Ercolani et al., J. Biol. Chem., 263:15335-15341,1988.
Evans, et al., In: Cancer Principles and Practice of Oncology, Devita et al. (Eds.), Lippincot-Raven, N.Y., 1054-1087, 1997.Fechheimer et al., Proc Natl. Acad. Sci. USA, 84:8463-8467, 1987.
Fraley et al., Proc. Natl. Acad. Sci. USA, 76:3348-3352, 1979.
Gaj et al., Trends in Biotechnology, 2013, 31(7), 397-405
Graham and Van Der Eb, Virology, 52:456-467, 1973.
International Publication No. WO 02/016536
International Publication No. WO 03/016496
International Publication No. WO 2003/0211603
International Publication No. WO 2007/069666
International Publication No. WO 2007/069666
International Publication No. WO 2012/0196360
International Publication No. WO 94/09699
International Publication No. WO 95/06128
International Publication No. WO 98/30679
International Publication No. WO 98/53058
International Publication No. WO 98/53059
International Publication No. WO 98/53060
Kaeppler et al., Plant Cell Reports 9: 415-418, 1990.
Kaneda et al., Science, 243:375-378, 1989.
Karin et al. Cell, 36:371-379,1989.
Kato et al, J. Biol. Chem., 266:3361-3364, 1991.
Kyttala et al., Stem Cell Reports, 6(2):200-12, 2016.
Langle-Rouault et al., J. Virol., 72(7):6181-6185, 1998.
Levitskaya et al., Proc. Natl. Acad. Sci. USA, 94(23):12616-12621, 1997.
Ludwig et al., Nat. Biotechnol., 24:185-187, 2006b.
Ludwig et al., Nat. Methods, 3:637-646, 2006a.
Macejak and Sarnow, Nature, 353:90-94, 1991.
Maniatis, et al., Molecular Cloning, A Laboratory Manual, Cold Spring Harbor Press, Cold Spring Harbor, N.Y., 1988.
Mann et al., Cell, 33:153-159, 1983.
Nabel et al., Science, 244(4910):1342-1344, 1989.
Naldini et al., Science, 272(5259):263-267, 1996.
Ng, Nuc. Acid Res., 17:601-615, 1989.
Nicolas and Rubenstein, In: Vectors: A survey of molecular cloning vectors and their uses, Rodriguez and Denhardt, eds., Stoneham: Butterworth, pp. 494-513, 1988.
Nicolau and Sene, Biochim. Biophys. Acta, 721:185-190, 1982.
Nicolau et al., Methods Enzymol., 149:157-176, 1987.
Paskind et al., Virology, 67:242-248, 1975.
Pelletier and Sonenberg, Nature, 334(6180):320-325, 1988.
Potrykus et al., Mol. Gen. Genet., 199(2):169-177, 1985.
Potter et al., Proc. Natl. Acad. Sci. USA, 81:7161-7165, 1984.
Quitsche et al., J. Biol. Chem., 264:9539-9545, 1989.
Richards et al., Cell, 37:263-272, 1984.
Rippe, et al., Mol. Cell Biol., 10:689-695, 1990.
Sambrook and Russell, Molecular Cloning: A Laboratory Manual, 3rd Ed. Cold Spring Harbor 1997.
Takahashi et al., Cell, 131:861-872, 2007.
Temin, In: Gene Transfer, Kucherlapati (Ed.), NY, Plenum Press, 149-188, 1986.
Tur-Kaspa et al., Mol. Cell Biol., 6:716-718, 1986.
U.S. Pat. No. 4,683,202
U.S. Pat. No. 5,302,523
U.S. Pat. No. 5,322,783
U.S. Pat. No. 5,384,253
U.S. Pat. No. 5,464,765
U.S. Pat. No. 5,538,877
U.S. Pat. No. 5,538,880
U.S. Pat. No. 5,550,318
U.S. Pat. No. 5,556,954
U.S. Pat. No. 5,563,055
U.S. Pat. No. 5,563,055
U.S. Pat. No. 5,580,859
U.S. Pat. No. 5,589,466
U.S. Pat. No. 5,591,616
U.S. Pat. No. 5,610,042
U.S. Pat. No. 5,656,610
U.S. Pat. No. 5,702,932
U.S. Pat. No. 5,736,524
U.S. Pat. No. 5,780,448
U.S. Pat. No. 5,789,215
U.S. Pat. No. 5,925,565
U.S. Pat. No. 5,928,906
U.S. Pat. No. 5,935,819
U.S. Pat. No. 5,945,100
U.S. Pat. No. 5,981,274
U.S. Pat. No. 5,994,136
U.S. Pat. No. 5,994,136
U.S. Pat. No. 5,994,624
U.S. Pat. No. 6,013,516
U.S. Pat. No. 6,103,470
U.S. Pat. No. 6,140,081
U.S. Pat. No. 6,416,998
U.S. Pat. No. 6,453,242
U.S. Pat. No. 6,534,261
U.S. Pat. No. 7,442,548
U.S. Pat. No. 7,598,364
U.S. Pat. No. 7,989,425
U.S. Pat. No. 8,058,065
U.S. Pat. No. 8,071,369
U.S. Pat. No. 8,129,187
U.S. Pat. No. 8,268,620
U.S. Pat. No. 8,278,620
U.S. Pat. No. 8,546,140
U.S. Pat. No. 8,546,140
U.S. Pat. No. 8,741,648
U.S. Patent Publication No. 2002/0076747
U.S. Patent Publication No. 20020055144
U.S. Patent Publication No. 2005/0064474
U.S. Patent Publication No. 2006/0188987
U.S. Patent Publication No. 2007/0218528
U.S. Patent Publication No. 20090148425
U.S. Patent Publication No. 20090246875
U.S. Patent Publication No. 2010/0003757
U.S. Patent Publication No. 2010/0210014
U.S. Patent Publication No. 2011/0301073
U.S. Patent Publication No. 2011/0301073
U.S. Patent Publication No. 20120276636
Wilson et al., Science, 244:1344-1346, 1989.
Wong et al., Gene, 10:87-94, 1980.
Yamanaka et al., Cell, 131(5):861-72, 2007.
Zufferey et al., Nat. Biotechnol., 15(9):871-875, 1997.

1. An isolated cell line engineered to express at least one transgene wherein the at least one transgene (a) is under the control of a promoter having at least 90% sequence identity to SEQ ID NOs:1-12 or 17; (b) is under the control of an endogenous gene selected from the group consisting of HSP90AB1, ACTB, CTNNB1, MYL6, UBA52, CAG, RPS, and UBC; and/or (c) is encoded by a sequence modified to remove CpG motifs to provide for stable expression.

2. The cell line of claim 1, wherein the at least one transgene (a) is under the control of a promoter having at least 90% sequence identity to SEQ ID NOs:1-12 or 17; and/or (b) is under the control of an endogenous gene selected from the group consisting of HSP90AB1, ACTB, CTNNB1, MYL6, UBA52, CAG, RPS, and UBC.

3. The cell line of claim 2, wherein the at least one transgene is encoded by a sequence modified to remove CpG motifs to provide for stable expression.

4. The cell line of claim 3, wherein the sequence modified to remove CpG motifs to provide for stable expression has at least 90% sequence identity to SEQ ID NO:14 or SEQ ID NO:16.

5. The cell line of claim 3, wherein the sequence modified to remove CpG motifs to provide for stable expression is SEQ ID NO:14 or SEQ ID NO:16.

6. The cell line of claim 1, wherein the at least one transgene is encoded by a sequence modified to remove CpG motifs to provide for stable expression and is under the control of a promoter having at least 90% sequence identity to SEQ ID NOs:1-12 or 17.

7. The cell line of claim 1, wherein the at least one transgene is encoded by a sequence modified to remove CpG motifs to provide for stable expression and is under the control of an endogenous gene selected from the group consisting of HSP90AB1, ACTB, CTNNB1, MYL6, UBA52, CAG, RPS, and UBC.

8. (canceled)

9. The cell line of claim 1, wherein the cell line is engineered to express at least a first transgene and a second transgene.

10. The cell line of claim 9, wherein the first transgene is under the control of a promoter having at least 90% sequence identity to SEQ ID NOs:1-12 or 17 and the second transgene is under the control of an endogenous gene selected from the group consisting of HSP90AB1, ACTB, CTNNB1, MYL6, UBA52, CAG, RPS, and UBC.

11. (canceled)

12. The cell line of claim 9, wherein the first transgene and/or second transgene are encoded by a sequence modified to remove CpG motifs for stable expression.

13. The cell line of claim 1, wherein at least 50 percent of the CpG motifs are removed.

14. (canceled)

15. (canceled)

16. The cell line of claim 1, wherein all CpG motifs are removed.

17. The cell line of claim 1, wherein the CpG motif codons are replaced with codons that are not rare and/or do not generate a mononucleotide stretch.

18. The cell line of claim 1, wherein the CpG motif codons are replaced with corresponding codons in Table 1.

19. The cell line of claim 1, wherein the cell line is an induced pluripotent stem cell (iPSC) line.

20. The cell line of claim 1, wherein the transgene is a reporter gene, suicide gene, or selection marker.

21-27. (canceled)

28. The cell line of claim 1, wherein the cell line has stable expression of the transgene over six months.

29. (canceled)

30. (canceled)

31. The cell line of claim 1, wherein the expression cassette is inserted at a genomic safe harbor site.

32. The cell line of claim 31, wherein the genomic safe harbor site is the PPP1R12C (AAVS1) locus or ROSA locus.

33. The cell line of claim 1, wherein the promoter has at least 90% sequence identity to SEQ ID NO: 2, 3, 4, 6, or 17.

34. (canceled)

35. (canceled)

36. The cell line of claim 1, wherein the promoter is a response element.

37. (canceled)

38. (canceled)

39. (canceled)

40. A method to prevent silencing of transgene expression in an engineered cell line comprising optimizing the transgene sequence to remove CpG motifs.

41-64. (canceled)

65. An expression vector comprising a promoter having at least 90% sequence identity to SEQ ID NOs: 1-12 or 17.

66-102. (canceled)
